(19) United States
(12) Patent Application Publication
(10) Pub. No.: US 2009/0291942 A1
(43) Pub. Date: Nov. 26, 2009

(54) IMIDAZO PYRIDINE DERIVATIVES

(76) Inventors: Ivan Cornella Taracido, Somerville, MA (US); Edmund Martin Harrington, Plymouth, MA (US); René Hersperger, Basel (CH); René Lattmann, Basel (CH); Wolfgang Miltz, Basel (CH); Klaus Weigand, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/468,706

(22) Filed: May 19, 2009

(30) Foreign Application Priority Data
May 26, 2008 (EP) 08156933.7

(51) Int. Cl. A61K 31/55; A61K 31/5377; A61K 31/497; A61K 31/437; C07D 471/04

(52) U.S. Cl. 514/217.07; 514/234.2; 514/253.04; 514/303; 540/597; 544/127; 544/362; 546/118

(57) ABSTRACT

The invention relates to novel imidazopyridine derivatives and to their use in the treatment of diseases and disorders which may e.g. involve angiogenesis and/or pain, including autoimmune and inflammatory diseases.

The invention relates to imidazopyridine derivatives. These appear to be useful as modulators e.g. antagonists of the GPR4 receptor and hence may typically have use in the treatment of diseases and disorders which for example involve angiogenesis and/or pain, or which pertain for example to autoimmune and/or inflammatory diseases and disorders.

Accordingly the invention in a first aspect provides a compound of formula (I), or a pharmaceutically acceptable salt thereof;

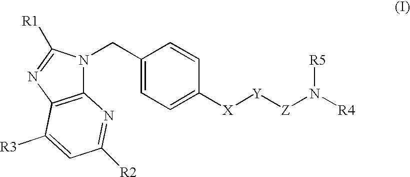

wherein

R1 is lower alkyl optionally substituted by halogen;

R2 and R3 are independently selected from H and lower alkyl;

X—Y stands for —C≡C—, or —CH═CH—, —CH═CHF—, —CH₂—CH₂—, —NHCO—, —CONH—;

Z is —CH₂—, —CH₂—CH₂—, —CH₂—CH₂—CH₂—CH₂—, —CO—, bond;

R4 is H or lower alkyl and R5 is selected from lower alkyl substituted by heterocyclyl;

or R4 and R5 together with the nitrogen atom to which they are attached form a heterocyclic ring;

or R4 and R5 together with the nitrogen atom to which they are attached form a heteroaryl.

In another embodiment in a compound of formula (I)

R1 is lower alkyl optionally substituted by halogen;

R2 and R3 are independently selected from H and lower alkyl;

X—Y stands for —C≡C—, or —CH═CH—, —CH═CHF—, —CH₂—CH₂—, —NHCO—, —CONH—;

Z is —CH₂—, —CH₂—CH₂—, —CH₂—CH₂—CH₂—CH₂—, —CO—, bond;

R4 is H or lower alkyl and R5 is selected from lower alkyl substituted by heterocyclyl;

or R4 and R5 together with the nitrogen atom to which they are attached form a heterocyclic ring which is optionally substituted by lower alkoxy; lower alkoxy substituted by (lower)alkylaminocarbonyl; hydroxyl; di-lower alkyl amino; heterocyclyl; or by lower alkyl optionally substituted by halogen, carbamoyl, alkoxycarbonyl, alkoxycarbonyl amino, hydroxyl, lower alkoxy, amino, di-lower alkyl amino, di-lower alkyl aminocarbonyl, cycloalkyl, aryl or heterocyclyl;

or R4 and R5 together with the nitrogen atom to which they are attached form a heteroaryl.

In another embodiment in a compound of formula (I)

R1 is C₁-C₄ alkyl optionally substituted by fluoro; R1 is in particular trifluoromethyl, methyl, ethyl, n-propyl, n-butyl;

R2 and R3 are independently selected from C₁-C₄ alkyl; in particular methyl;

X—Y stands for —CH₂—CH₂—; —C≡C—, or —CH═CH—;

Z is —CH₂— or —CH₂—CH₂—; in particular —CH₂—;

R4 and R5 together with the nitrogen atom to which they are attached may form a 4-10 membered saturated, or unsaturated heterocyclic ring optionally containing up to 2 ring members selected from CHNR6R7, N, NH, O, and NC₁-C₆ alkyl optionally substituted by hydroxyl, C₁-C₆ alkoxy, amino, or di-C₁-C₄ alkyl amino;

R6 and R7 are independently selected from hydrogen and alkyl, or

R6 and R7 together with the nitrogen atom to which they are attached may form a 4-7 membered saturated heterocyclic ring optionally containing a ring member selected from CHNR6R7, O, NH, NC₁-C₆ alkyl optionally substituted by hydroxyl, C₁-C₆ alkoxy, amino, or di-C₁-C₄ alkyl amino; wherein R6 and R7 have the meanings provided above.

In another embodiment in a compound of formula (I)

R1 is C₁-C₄ alkyl; in particular ethyl;

R2 and R3 are independently selected from C₁-C₂ alkyl; in particular methyl;

X—Y stands for —CH₂—CH₂—; —C≡C—, or —CH═CH—;

Z is —CH₂— or —CO—;

R4 is H and R5 is selected from lower alkyl substituted by heterocyclyl,

or R4 and R5 together with the nitrogen atom to which they are attached form a piperidine or a piperazin ring which is optionally substituted in position 4 by C₁-C₆ alkyl, di-C₁-C₄ alkyl amino, 4-C₁-C₆-alkyl-piperazin-1-yl, 4-C₁-C₆-alkyloxy(lower)alkyl-piperazin-1-yl, 4-C₁-C₆-dialkylamino(lower)alkyl-piperazin-1-yl, 1-morpholinyl, 1-piperidinyl, 1-pyrrolidinyl.

In another embodiment in a compound of formula (I)

R1 is C₁-C₄ alkyl; in particular methyl or ethyl;

R2 and R3 are independently selected from C₁-C₂ alkyl; in particular methyl;

X—Y stands for —CH₂—CH₂—; or —CH═CH—;

Z is —CH₂— or —CH₂—CH₂—;

R4 and R5 together with the nitrogen atom to which they are attached form a piperidine or a piperazin ring which is optionally substituted in position 4 by C₁-C₆ alkyl, di-C₁-C₄ alkyl amino, 4-C₁-C₆-alkyl-piperazin-1-yl, 4-C₁-C₆-alkyloxy(lower)alkyl-piperazin-1-yl, 4-C₁-C₆-dialkylamino(lower)alkyl-piperazin-1-yl, 1-morpholinyl, 1-piperidinyl, 1-pyrrolidinyl; or

R4 and R5 together with the nitrogen atom to which they are attached form heteroaryl.

In a compound of formula (I), the following significances provide further embodiments of the invention, independently, collectively or in any combination or sub-combination thereof:
1. R1 is methyl;
2. R1 is ethyl;
3. R1 is trifluoromethyl;
4. R1 is propyl;
5. R2 is methyl;
6. R3 is methyl;
7. X—Y stands for —CH═CH—;
8. X—Y stands for —C≡C—;
9. X—Y stands for —NHCO—,
10. X—Y stands for —CONH—;
11. X—Y stands for —CH═CH— and Z is —CH₂—;
12. Z is —CH₂—;
13. Z is —(CH₂)₂—;
14. Z is —(CH₂)₄—;
15. Z is CO;
16. N R4 R5 is 4-C₁-C₆-alkyl-piperazin-1-yl;
17. N R4 R5 is 4-morpholin-4-yl-piperidin-1-yl;
18. N R4 R5 is 4-pyrrolidin-1-yl-piperidin-1-yl;
19. N R4 R5 is 4-piperidin-1-yl-piperidin-1-yl (or [1,4′]bipiperidinyl); or
20. N R4 R5 is imidazol-1-yl;

For the avoidance of doubt, the terms listed below are to be understood to have the following meaning throughout the present description and claims:

Alkyl as used herein includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.). If not indicated otherwise an alkyl contains from 1 to 18 carbon atoms, in particular from 1 to 12 carbon atoms. In another aspect alkyl refers to lower alkyl.

The term “lower”, when referring to organic radicals or compounds means a compound or radical with may be branched or unbranched with up to and including 7 carbon atoms.

A lower alkyl group may be branched, unbranched or cyclic and contains 1 to 7 carbon atoms, preferably 1 to 4 carbon atoms. Lower alkyl represents, for example: methyl, ethyl, propyl, butyl, isopropyl, isobutyl, tertiary butyl or 2,2-dimethylpropyl.

A lower alkoxy group may be branched or unbranched and contains 1 to 7 carbon atoms, preferably 1 to 6 carbon atoms. Lower alkoxy represents, for example: methoxy, ethoxy, propoxy, butoxy, isopropoxy, isobutoxy or tertiary butoxy. Lower alkoxy includes cycloalkyloxy and cycloalkyl—lower alkyloxy.

A lower alkene, alkenyl or alkenoxy group is branched or unbranched and contains 2 to 7 carbon atoms, preferably 1 to 4 carbon atoms and contains at least one carbon-carbon double bond. Lower alkene, lower alkenyl or lower alkenyloxy represents for example vinyl, prop-1-enyl, allyl, butenyl, isopropenyl or isobutenyl and the oxy equivalents thereof.

In the present application, oxygen containing substituents, e.g. alkoxy, alkenyloxy, alkynyloxy, carbonyl, etc. encompass their sulphur containing homologues, e.g. thioalkoxy, thioalkenyloxy, thioalkynyloxy, thiocarbonyl, sulphone, sulphoxide etc.

The term “aryl” refers to an aromatic hydrocarbon group having 6-20 carbon atoms in the ring portion. Preferably, aryl is monocyclic, bicyclic or tricyclic having 6-20 carbon atoms.

Furthermore, the term “aryl” as used herein, refers to an aromatic substituent which can be a single aromatic ring, or multiple aromatic rings that are fused together, linked covalently, or linked to a common group such as a methylene or ethylene moiety. The common linking group also can be a carbonyl as in benzophenone or oxygen as in diphenylether or nitrogen as in diphenylamine.

Non-limiting examples include phenyl, biphenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamido, heterocyclyl and the like.

As used herein, the term “heterocyclic”, “heterocyclyl” or “heterocyclo” refers to an optionally substituted, saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, azepin, oxathiolane, dithiolane, 1,3-dioxane, 1,3-dithiane, oxathiane, thiomorpholine, and include also fused and bridged heterocycloalkyl groups such as 3-heteroaryl-8-aza-bicyclo[3.2.1]oct-8-yl, 8-aza-bicyclo[3.2.1]oct-8-yl, and the like.

The term “heterocyclic” further refers to heterocyclic groups as defined herein substituted with 1, 2 or 3 substituents selected from the groups consisting of the following:
(a) lower alkyl;
(b) hydroxy (or protected hydroxy);
(c) halo;
(d) oxo, i.e., ═O;
(e) amino, lower alkylamino or lower dialkylamino;
(f) lower alkoxy;
(g) cycloalkyl;
(h) carboxyl;
(i) heterocyclooxy, wherein heterocyclooxy denotes a heterocyclic group bonded through an oxygen bridge;
(j) lower alkyl-O—C(O)—;
(k) aryl optionally substituted with lower alkyl, cycloalkyl, lower alkoxy, hydroxy, amino, lower alkyl-C(O)—NH—, lower alkylamino, lower dialkylamino or halogen;
(l) lower alkyl-C(O)—O—;
(m) aryl-C(O)—O—;
(n) aryloxy;
(m) formyl, i.e., HC(O)—;
(o) carbamoyl;
(p) aryl-lower alkyl-.

As used herein, the term “cycloalkyl” refers to saturated or unsaturated monocyclic, bicyclic or tricyclic hydrocarbon groups of 3-12 carbon atoms, preferably 3-9, or 3-7 carbon atoms, each of which can be optionally substituted by one, or two, or three, or more substituents, such as alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, heterocyclyl and the like. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, (aryl-alkyl)-NHS(O)₂—, (heteroaryl-alkyl)-NHS(O)₂— and the like.

As used herein, the term “aryloxy” refers to both an —O-aryl and an —O-heteroaryl group, wherein aryl and heteroaryl are defined herein.

As used herein, the term “heteroaryl” refers to a 5-14 membered monocyclic- or bicyclic- or polycyclic-aromatic ring system, having 1 to 8 heteroatoms selected from N, O or S. Preferably, the heteroaryl is a 5-10 or 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or I—, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

A heteroaryl group may be mono-, bi-, tri-, or polycyclic, preferably mono-, bi-, or tricyclic, more preferably mono- or bicyclic.

As used herein, the term “halogen” or “halo” refers to fluoro, chloro, bromo, and iodo.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)— or (S)—. The present invention is meant to include all such possible isomers, including racemic mixtures, optically pure forms and intermediate mixtures. Optically active (R)— and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, sodium, potassium, ammonium, calcium, magnesium, iron, silver, zinc, copper and the like; particularly preferred are the ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The present invention includes all pharmaceutically acceptable isotopically-labeled compounds of the invention, i.e. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to at least partially alleviating, inhibiting, preventing and/or ameliorating a condition, or a disorder or a disease as described in the corresponding section of the present invention.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a primate. In another preferred embodiment, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)—, (S)— or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)— or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization.

In another embodiment the invention provides a compound of formula I being:

2-Ethyl-3-{4-[3-(4-isopropyl-piperazin-1-yl)-prop-1-ynyl]-benzyl}-5,7-dimethyl-3H-imidazo[4,5-b]pyridine

1′-{3-[4-(2-Ethyl-5,7-dimethyl-imidazo[4,5-b]pyridin-3-ylmethyl)-phenyl]-prop-2-ynyl}-[1,4′]bipiperidine

2-Ethyl-5,7-dimethyl-3-[4-(3-pyrrolidin-1-yl-prop-1-ynyl)-benzyl]-3H-imidazo[4,5-b]pyridine

{3-[4-(2-Ethyl-5,7-dimethyl-imidazo[4,5-b]pyridin-3-ylmethyl)-phenyl]-prop-2-ynyl}-(2-pyrrolidin-1-yl-ethyl)-amine

1′-{2-[4-(2-Ethyl-5,7-dimethyl-imidazo[4,5-b]pyridin-3-ylmethyl)-phenyl]-ethyl}-[1,4′]bipiperidinyl

2-Ethyl-5,7-dimethyl-3-{4-[3-(4-methyl-piperazin-1-yl)-propyl]-benzyl}-3H-imidazo[4,5-b]pyridine

2-Ethyl-5,7-dimethyl-3-{4-[3-(4-propyl-piperazin-1-yl)-propyl]-benzyl}-3H-imidazo[4,5-b]pyridine

2-Ethyl-3-{4-[3-(4-isopropyl-piperazin-1-yl)-propyl]-benzyl}-5,7-dimethyl-3H-imidazo[4,5-b]pyridine

3-{4-[3-(4-Cyclopropyl-piperazin-1-yl)-propyl]-benzyl}-2-ethyl-5,7-dimethyl-3H-imidazo[4,5-b]pyridine

3-{4-[3-(4-Butyl-piperazin-1-yl)-propyl]-benzyl}-2-ethyl-5,7-dimethyl-3H-imidazo[4,5-b]pyridine

3-{4-[3-(4-sec-Butyl-piperazin-1-yl)-propyl]-benzyl}-2-ethyl-5,7-dimethyl-3H-imidazo[4,5-b]pyridine

3-{4-[3-(4-tert-Butyl-piperazin-1-yl)-propyl]-benzyl}-2-ethyl-5,7-dimethyl-3H-imidazo[4,5-b]pyridine

2-Ethyl-5,7-dimethyl-3-{4-[3-(4-pentyl-piperazin-1-yl)-propyl]-benzyl}-3H-imidazo[4,5-b]pyridine

2-Ethyl-5,7-dimethyl-3-(4-{3-[4-(1-methyl-butyl)-piperazin-1-yl]-propyl}-benzyl)-3H-imidazo[4,5-b]pyridine

2-Ethyl-3-(4-{3-[4-(1-ethyl-propyl)-piperazin-1-yl]-propyl}-benzyl)-5,7-dimethyl-3H-imidazo[4,5-b]pyridine

2-Ethyl-5,7-dimethyl-3-(4-{3-[4-(3-methyl-butyl)-piperazin-1-yl]-propyl}-benzyl)-3H-imidazo[4,5-b]pyridine

3-{4-[3-(4-Cyclobutylmethyl-piperazin-1-yl)-propyl]-benzyl}-2-ethyl-5,7-dimethyl-3H-imidazo[4,5-b]pyridine

3-{4-[3-(4-Cyclopentyl-piperazin-1-yl)-propyl]-benzyl}-2-ethyl-5,7-dimethyl-3H-imidazo[4,5-b]pyridine

3-{4-[3-(4-Cyclohexyl-piperazin-1-yl)-propyl]-benzyl}-2-ethyl-5,7-dimethyl-3H-imidazo[4,5-b]pyridine

2-Ethyl-3-(4-{3-[4-(2-methoxy-ethyl)-piperazin-1-yl]-propyl}-benzyl)-5,7-dimethyl-3H-imidazo[4,5-b]pyridine

[2-(4-{3-[4-(2-Ethyl-5,7-dimethyl-imidazo[4,5-b]pyridin-3-ylmethyl)-phenyl]-propyl}-piperazin-1-yl)-ethyl]-dimethyl-aminee

2-Ethyl-3-{4-[3-(4-ethyl-3-methyl-piperazin-1-yl)-propyl]-benzyl}-5,7-dimethyl-3H-imidazo[4,5-b]pyridine

2-Ethyl-3-{4-[3-(4-isopropyl-3-methyl-piperazin-1-yl)-propyl]-benzyl}-5,7-dimethyl-3H-imidazo[4,5-b]pyridine

2-Ethyl-3-{4-[3-((S)-2-isopropyl-4-methyl-piperazin-1-yl)-propyl]-benzyl}-5,7-dimethyl-3H-imidazo[4,5-b]pyridine

2-Ethyl-3-{4-[3-(3-ethyl-4-isopropyl-piperazin-1-yl)-propyl]-benzyl}-5,7-dimethyl-3H-imidazo[4,5-b]pyridine

2-Ethyl-3-{4-[3-((1R,4R)-5-ethyl-2,5-diaza-bicyclo[2.2.1]hept-2-yl)-propyl]-benzyl}-5,7-dimethyl-3H-imidazo[4,5-b]pyridine

2-Ethyl-3-{4-[3-((1R,4R)-5-isobutyl-2,5-diaza-bicyclo[2.2.1]hept-2-yl)-propyl]-benzyl}-5,7-dimethyl-3H-imidazo[4,5-b]pyridine

3-{4-[3-((1R,4R)-5-Cyclopropylmethyl-2,5-diaza-bicyclo[2.2.1]hept-2-yl)-propyl]-benzyl}-2-ethyl-5,7-dimethyl-3H-imidazo[4,5-b]pyridine

3-{4-[(E)-3-(4-Isopropyl-piperazin-1-yl)-propenyl]-benzyl}-2,5,7-trimethyl-3H-imidazo[4,5-b]pyridine

1′-{(E)-3-[4-(2,5,7-Trimethyl-imidazo[4,5-b]pyridin-3-ylmethyl)-phenyl]-allyl}-[1,4′]bipiperidinyl

Diethyl-(1-{(E)-3-[4-(2-ethyl-5,7-dimethyl-imidazo[4,5-b]pyridin-3-ylmethyl)-phenyl]-allyl}-piperidin-4-yl)-amine

2-Ethyl-5,7-dimethyl-3-{4-[(E)-3-(4-pyrrolidin-1-yl-piperidin-1-yl)-propenyl]-benzyl}-3H-imidazo[4,5-b]pyridine

1′-{(E)-3-[4-(2-Ethyl-5,7-dimethyl-imidazo[4,5-b]pyridin-3-ylmethyl)-phenyl]-allyl}-[1,4′]bipiperidinyl

2-Ethyl-5,7-dimethyl-3-{4-[(E)-3-(4-morpholin-4-yl-piperidin-1-yl)-propenyl]-benzyl}-3H-imidazo[4,5-b]pyridine

2-Ethyl-5,7-dimethyl-3-(4-{(E)-3-[4-(4-methyl-piperazin-1-yl)-piperidin-1-yl]-propenyl}-benzyl)-3H-imidazo[4,5-b]pyridine

1-{(E)-3-[4-(2-Ethyl-5,7-dimethyl-imidazo[4,5-b]pyridin-3-ylmethyl)-phenyl]-allyl}-piperidin-4-ol

2-Ethyl-3-{4-[(E)-3-(4-methoxy-piperidin-1-yl)-propenyl]-benzyl}-5,7-dimethyl-3H-imidazo[4,5-b]pyridine

2-(1-{(E)-3-[4-(2-Ethyl-5,7-dimethyl-imidazo[4,5-b]pyridin-3-ylmethyl)-phenyl]-allyl}-piperidin-4-yloxy)-N-methyl-acetamide

1-{(E)-3-[4-(2-Ethyl-5,7-dimethyl-imidazo[4,5-b]pyridin-3-ylmethyl)-phenyl]-allyl}-4-morpholin-4-ylmethyl-piperidin-4-ol

2-Ethyl-3-(4-{(E)-3-[4-(1H-indol-3-yl)-piperidin-1-yl]-propenyl}-benzyl)-5,7-dimethyl-3H-imidazo[4,5-b]pyridine

3-{4-[(E)-3-((1R,5S)-3-Benzoimidazol-1-yl-8-aza-bicyclo[3.2.1]oct-8-yl)-propenyl]-benzyl}-2-ethyl-5,7-dimethyl-3H-imidazo[4,5-b]pyridine

2-Ethyl-5,7-dimethyl-3-[4-((E)-3-piperazin-1-yl-propenyl)-benzyl]-3H-imidazo[4,5-b]pyridine

2-Ethyl-5,7-dimethyl-3-{4-[(E)-3-(4-methyl-piperazin-1-yl)-propenyl]-benzyl}-3H-imidazo[4,5-b]pyridine

2-Ethyl-3-{4-[(E)-3-(4-ethyl-piperazin-1-yl)-propenyl]-benzyl}-5,7-dimethyl-3H-imidazo[4,5-b]pyridine

2-Ethyl-5,7-dimethyl-3-{4-[(E)-3-(4-propyl-piperazin-1-yl)-propenyl]-benzyl}-3H-imidazo[4,5-b]pyridine

2-Ethyl-3-{4-[(E)-3-(4-isopropyl-piperazin-1-yl)-propenyl]-benzyl}-5,7-dimethyl-3H-imidazo[4,5-b]pyridine

3-{4-[(E)-3-(4-Cyclopropyl-piperazin-1-yl)-propenyl]-benzyl}-2-ethyl-5,7-dimethyl-3H-imidazo[4,5-b]pyridine

3-{4-[(E)-3-(4-Butyl-piperazin-1-yl)-propenyl]-benzyl}-2-ethyl-5,7-dimethyl-3H-imidazo[4,5-b]pyridine

3-{4-[(E)-3-(4-sec-Butyl-piperazin-1-yl)-propenyl]-benzyl}-2-ethyl-5,7-dimethyl-3H-imidazo[4,5-b]pyridine

2-Ethyl-3-{4-[(E)-3-(4-isobutyl-piperazin-1-yl)-propenyl]-benzyl}-5,7-dimethyl-3H-imidazo[4,5-b]pyridine

3-{4-[(E)-3-(4-tert-Butyl-piperazin-1-yl)-propenyl]-benzyl}-2-ethyl-5,7-dimethyl-3H-imidazo[4,5-b]pyridine

2-Ethyl-5,7-dimethyl-3-(4-{(E)-3-[4-(3-methyl-butyl)-piperazin-1-yl]-propenyl}-benzyl)-3H-imidazo[4,5-b]pyridine

2-Ethyl-3-(4-{(E)-3-[4-(1-ethyl-propyl)-piperazin-1-yl]-propenyl}-benzyl)-5,7-dimethyl-3H-imidazo[4,5-b]pyridine

2-Ethyl-5,7-dimethyl-3-(4-{(E)-3-[4-(1-methyl-butyl)-piperazin-1-yl]-propenyl}-benzyl)-3H-imidazo[4,5-b]pyridine

3-{4-[(E)-3-(4-Cyclobutylmethyl-piperazin-1-yl)-propenyl]-benzyl}-2-ethyl-5,7-dimethyl-3H-imidazo[4,5-b]pyridine

3-{4-[(E)-3-(4-Cyclopentyl-piperazin-1-yl)-propenyl]-benzyl}-2-ethyl-5,7-dimethyl-3H-imidazo[4,5-b]pyridine

3-{4-[(E)-3-(4-Cyclohexyl-piperazin-1-yl)-propenyl]-benzyl}-2-ethyl-5,7-dimethyl-3H-imidazo[4,5-b]pyridine

[2-(4-{(E)-3-[4-(2-Ethyl-5,7-dimethyl-imidazo[4,5-b]pyridin-3-ylmethyl)-phenyl]-allyl}-piperazin-1-yl)-ethyl]-dimethyl-amine

[3-(4-{(E)-3-[4-(2-Ethyl-5,7-dimethyl-imidazo[4,5-b]pyridin-3-ylmethyl)-phenyl]-allyl}-piperazin-1-yl)-propyl]-dimethyl-amine

Diethyl-[2-(4-{(E)-3-[4-(2-ethyl-5,7-dimethyl-imidazo[4,5-b]pyridin-3-ylmethyl)-phenyl]-allyl}-piperazin-1-yl)-ethyl]-amine

[2-(4-{(E)-3-[4-(2-Ethyl-5,7-dimethyl-imidazo[4,5-b]pyridin-3-ylmethyl)-phenyl]-allyl}-piperazin-1-yl)-ethyl]-diisopropyl-amine

2-Ethyl-3-(4-{(E)-3-[4-(2-methoxy-ethyl)-piperazin-1-yl]-propenyl}-benzyl)-5,7-dimethyl-3H-imidazo[4,5-b]pyridine

3-(4-{(E)-3-[4-(2-Ethoxy-ethyl)-piperazin-1-yl]-propenyl}-benzyl)-2-ethyl-5,7-dimethyl-3H-imidazo[4,5-b]pyridine

2-Ethyl-3-(4-{(E)-3-[4-(3-methoxy-propyl)-piperazin-1-yl]-propenyl}-benzyl)-5,7-dimethyl-3H-imidazo[4,5-b]pyridine

2-(4-{(E)-3-[4-(2-Ethyl-5,7-dimethyl-imidazo[4,5-b]pyridin-3-ylmethyl)-phenyl]-allyl}-piperazin-1-yl)-N,N-dimethyl-acetamide

2-Ethyl-5,7-dimethyl-3-(4-{(E)-3-[4-(2-morpholin-4-yl-ethyl)-piperazin-1-yl]-propenyl}-benzyl)-3H-imidazo[4,5-b]pyridine

3-(4-{(E)-3-[4-(3,4-Dimethoxy-benzyl)-piperazin-1-yl]-propenyl}-benzyl)-2-ethyl-5,7-dimethyl-3H-imidazo[4,5-b]pyridine

2-Ethyl-3-(4-{(E)-3-[4-(3-fluoro-propyl)-piperazin-1-yl]-propenyl}-benzyl)-5,7-dimethyl-3H-imidazo[4,5-b]pyridine

2-Ethyl-3-{4-[(E)-3-(4-ethyl-3-methyl-piperazin-1-yl)-propenyl]-benzyl}-5,7-dimethyl-3H-imidazo[4,5-b]pyridine

2-Ethyl-3-{4-[(E)-3-((S)-2-isopropyl-4-methyl-piperazin-1-yl)-propenyl]-benzyl}-5,7-dimethyl-3H-imidazo[4,5-b]pyridine

2-Ethyl-3-{4-[(E)-3-(4-isopropyl-3-methyl-piperazin-1-yl)-propenyl]-benzyl}-5,7-dimethyl-3H-imidazo[4,5-b]pyridine

2-Ethyl-3-{4-[(E)-3-(3-ethyl-4-isopropyl-piperazin-1-yl)-propenyl]-benzyl}-5,7-dimethyl-3H-imidazo[4,5-b]pyridine

2-Ethyl-5,7-dimethyl-3-{4-[(E)-3-(1-methyl-hexahydro-pyrrolo[1,2-a]pyrazin-2-yl)-propenyl]-benzyl}-3H-imidazo[4,5-b]pyridine

2-Ethyl-3-{4-[(E)-3-((1R,4R)-5-ethyl-2,5-diaza-bicyclo[2.2.1]hept-2-yl)-propenyl]-benzyl}-5,7-dimethyl-3H-imidazo[4,5-b]pyridine

2-Ethyl-3-{4-[(E)-3-((1R,4R)-5-isobutyl-2,5-diaza-bicyclo[2.2.1]hept-2-yl)-propenyl]-benzyl}-5,7-dimethyl-3H-imidazo[4,5-b]pyridine

3-{4-[(E)-3-((1R,4R)-5-Cyclopropylmethyl-2,5-diaza-bicyclo[2.2.1]hept-2-yl)-propenyl]-benzyl}-2-ethyl-5,7-dimethyl-3H-imidazo[4,5-b]pyridine

(4-{(E)-3-[4-(2-Ethyl-5,7-dimethyl-imidazo[4,5-b]pyridin-3-ylmethyl)-phenyl]-allyl}-morpholin-2-ylmethyl)-carbamic acid tert-butyl ester

C-(4-{(E)-3-[4-(2-Ethyl-5,7-dimethyl-imidazo[4,5-b]pyridin-3-ylmethyl)-phenyl]-allyl}-morpholin-2-yl)-methylamine

(4-{(E)-3-[4-(2-Ethyl-5,7-dimethyl-imidazo[4,5-b]pyridin-3-ylmethyl)-phenyl]-allyl}-morpholin-2-ylmethyl)-isopropyl-amine

2-Ethyl-3-[4-((E)-3-imidazol-1-yl-propenyl)-benzyl]-5,7-dimethyl-3H-imidazo[4,5-b]pyridine

3-{4-[(E)-3-(4-Isopropyl-piperazin-1-yl)-propenyl]-benzyl}-5,7-dimethyl-2-propyl-3H-imidazo[4,5-b]pyridine

1′-{(E)-3-[4-(5,7-Dimethyl-2-propyl-imidazo[4,5-b]pyridin-3-ylmethyl)-phenyl]-allyl}-[1,4′]bipiperidine

1′-{(E)-3-[4-(2-Isopropyl-5,7-dimethyl-imidazo[4,5-b]pyridin-3-ylmethyl)-phenyl]-allyl}-[1,4′]bipiperidinyl

2-Cyclopropyl-3-{4-[(E)-3-(4-isopropyl-piperazin-1-yl)-propenyl]-benzyl}-5,7-dimethyl-3H-imidazo[4,5-b]pyridine

2-Butyl-3-{4-[(E)-3-(4-isopropyl-piperazin-1-yl)-propenyl]-benzyl}-5,7-dimethyl-3H-imidazo[4,5-b]pyridine

1′-{(E)-3-[4-(2-Butyl-5,7-dimethyl-imidazo[4,5-b]pyridin-3-ylmethyl)-phenyl]-allyl}-[1,4′]bipiperidine

1′-{(E)-3-[4-(5,7-Dimethyl-2-trifluoromethyl-imidazo[4,5-b]pyridin-3-ylmethyl)-phenyl]-allyl}-[1,4′]bipiperidinyl

5,7-Dimethyl-3-(4-{(E)-3-[4-(4-methyl-piperazin-1-yl)-piperidin-1-yl]-propenyl}-benzyl)-2-trifluoromethyl-3H-imidazo[4,5-b]pyridine

5,7-Dimethyl-3-{4-[(E)-3-(4-morpholin-4-yl-piperidin-1-yl)-propenyl]-benzyl}-2-trifluoromethyl-3H-imidazo[4,5-b]pyridine

1-{(E)-3-[4-(5,7-Dimethyl-2-trifluoromethyl-imidazo[4,5-b]pyridin-3-ylmethyl)-phenyl]-allyl}-piperidine-4-carboxylic acid methyl ester

3-{4-[(E)-3-(4-Isopropyl-piperazin-1-yl)-propenyl]-benzyl}-5,7-dimethyl-2-trifluoromethyl-3H-imidazo[4,5-b]pyridine

3-{4-[(E)-3-(4-tert-Butyl-piperazin-1-yl)-propenyl]-benzyl}-5,7-dimethyl-2-trifluoromethyl-3H-imidazo[4,5-b]pyridine

1′-{(Z)-3-[4-(2-Ethyl-5,7-dimethyl-imidazo[4,5-b]pyridin-3-ylmethyl)-phenyl]-2-fluoro-allyl}-[1,4′]bipiperidinyl

2-Ethyl-3-{4-[(Z)-2-fluoro-3-(4-isopropyl-piperazin-1-yl)-propenyl]-benzyl}-5,7-dimethyl-3H-imidazo[4,5-b]pyridine

3-{4-[(Z)-3-(4-tert-Butyl-piperazin-1-yl)-2-fluoro-propenyl]-benzyl}-2-ethyl-5,7-dimethyl-3H-imidazo[4,5-b]pyridine

(E)-1-(4-Diethylamino-piperidin-1-yl)-3-[4-(2-ethyl-5,7-dimethyl-imidazo[4,5-b]pyridin-3-ylmethyl)-phenyl]-propenone

(E)-1-[1,4′]Bipiperidinyl-1′-yl-3-[4-(2-ethyl-5,7-dimethyl-imidazo[4,5-b]pyridin-3-ylmethyl)-phenyl]-propenone

(E)-3-[4-(2-Ethyl-5,7-dimethyl-imidazo[4,5-b]pyridin-3-ylmethyl)-phenyl]-1-(4-morpholin-4-yl-piperidin-1-yl)-propenone

4-(2-Ethyl-5,7-dimethyl-imidazo[4,5-b]pyridin-3-ylmethyl)-N-(4-pyrrolidin-1-yl-butyl)-benzamide

N-(4-Azepan-1-yl-butyl)-4-(2-ethyl-5,7-dimethyl-imidazo[4,5-b]pyridin-3-ylmethyl)-benzamide

5-Pyrrolidin-1-yl-pentanoic acid [4-(2-ethyl-5,7-dimethyl-imidazo[4,5-b]pyridin-3-ylmethyl)-phenyl]-amide

5-Piperidin-1-yl-pentanoic acid [4-(2-ethyl-5,7-dimethyl-imidazo[4,5-b]pyridin-3-ylmethyl)-phenyl]-amide

5-(3-Dimethylamino-pyrrolidin-1-yl)-pentanoic acid [4-(2-ethyl-5,7-dimethyl-imidazo[4,5-b]pyridin-3-ylmethyl)-phenyl]-amide, and/or

2-Ethyl-5,7-dimethyl-3-[4-(1-methyl-piperidin-3-ylmethoxy)-benzyl]-3H-imidazo[4,5-b]pyridine

According to another aspect of the invention there is provided a compound of formula (I) for use as a pharmaceutical for the prevention, amelioration or treatment of an autoimmune or inflammatory disease or condition.

Abbreviations:
celite Celite® (Diatomaceous Earth, World Minerals Inc., Santa Barbara, Calif., USA)
BINAP 2,2′-bis(diphenylphosphino)-1,1′-binaphthyl
DEAD: Diethyl azodicarboxylate
DIBAH: Di-isobutyl-aluminium-hydride
DIPEA: Diisopropylethylamine
DMEM: Dulbecco's Modified Eagle's Medium
DMF: N,N-Dimethyl formamide
DMSO: Dimethylsulfoxide
EtOAc: Acetic acid ethyl ester
HCl: Hydrochloric acid
HTRF: Homogenous time resolved fluorescence (assay)
IBMX: 3-Isobutyl-1-methyl-xanthine
rt: room temperature
RT Retention Time (in HPLC)
TBTU O-(1H-benzotriazol-1-yl)-N,N,N′,N′-tetramethyluronium tetrafluoroborate
THF: Tetrahydrofurane
TLC: Thin layer chromatography
VEGF: Vascular endothelial growth factor

1H-NMR spectra were recorded on a Varian Gemini 400 MHz NMR spectrometer. Significant peaks were tabulated in the order: multiplicity (s, singlet; d, doublet; t, triplet; q, quartet; m, multiplet; br, broad) and number of protons. Electron Spray Ionization (ESI) mass spectra were recorded on a Hewlett Packard 5989A mass spectrometer. Mass spectrometry results were reported as the ratio of mass over charge. Preparative HPLC purifications were performed with XTerra™ RP18 19×150 mm columns, using acetonitrile/water or MeOH/water as eluent systems. All reagents, starting materials and intermediates utilized in these examples were available from commercial sources or were readily prepared by methods known to those skilled in the art.

Synthesis of the imidazopyridine Building Blocks

The imidazopyridine building block 2-ethyl-5,7-dimethyl-3H-imidazo[4,5-b]pyridine 2b (R=ethyl) was synthesized as described by C. H. Senanayake et al (Heterocycles 1996, 42, 821-836). All other derivatives were prepared according to reaction scheme 1 and 2.

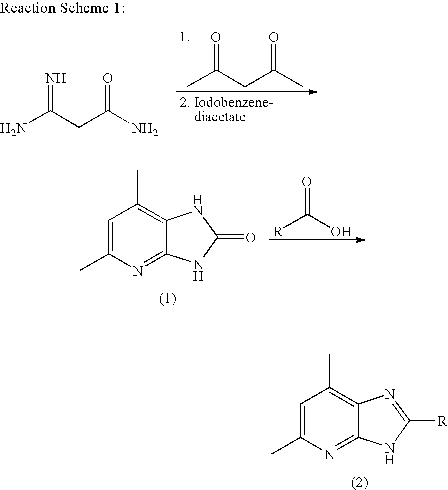

### Synthesis of 5,7-Dimethyl-1,3-dihydro-imidazo[4,5-b]pyridin-2-one (1)

Malonamidine (20.4 g, 148 mmol) was dissolved in 1 l of methanol and after addition of acetylacetone (15.3 ml, 148 mmol) and KOH pellets (10 g, 178 mmol) the mixture was stirred for 24 h at rt. 100 ml methanol was added followed by a solution of KOH (20.8 g, 370 mmol) in 100 ml of methanol. After 30 min stirring at rt the reaction mixture was cooled down to −5° C. Iodobenzene diacetate (47.8 g, 148 mmol) was added over 30 min at −5° C. The mixture was allowed to stir over night at rt. Then the mixture was filtrated of, washed with methanol and dried on HV. The product was used in the next step without further purification.

MS (ESI): 164 [M+H]⁺; ¹H-NMR (DMSO-d6, 400 MHz) δ (ppm): 6.38 (s, 1H), 4.0-6.0 (br, 2H), 2.28 (s, 3H), 2.17 (s, 3H).

### Synthesis of 2,5,7-Trimethyl-3H-imidazo[4,5-b]pyridine acetate (2a, R=methyl)

5,7-Dimethyl-1,3-dihydro-imidazo[4,5-b]pyridin-2-one (1) (23.8 g, 145 mmol) was dissolved in 300 ml of acetanhydride and after addition of acetic acid (163 ml, 2.8 mol) and MgCl₂ (13.9 g, 145 mmol) the mixture was stirred for 16 h (TLC control) at 100° C. Then the mixture was quenched with 30 ml methanol and evaporated. The residue was diluted with water and extracted 10 times with ethyl acetate. The combined organic layers were dried over Na₂SO₄ and evaporated under reduced pressure. The crude product was purified by recrystallization from ether.

MS (ESI): 162 [M+H]⁺; ¹H-NMR (DMSO-d6, 400 MHz) δ (ppm): 12.4 (br, 1H), 11.95 (br, 1H), 6.8 (s, 1H), 2.43 (s, 9H), 1.9 s, 3H).

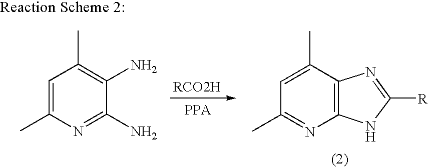

### Synthesis of 5,7-dimethyl-2-propyl-3H-imidazo[4,5-b]pyridine (2c, R=propyl)

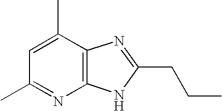

4,6-Dimethyl-pyridine-2,3-diamine (1 g, 7.3 mmol) and n-butyric acid (0.64 g, 7.3 mmol) were added to polyphosphoric acid (73 ml) and the mixture was heated to 190° C. After stirring at this temperature for 2 h (TLC control), the mixture was poured on ice and neutralized with K₂CO₃. After addition of ethyl acetate, the organic layer was separated and dried over Na₂SO₄. Evaporation gave a off-white solid.

MS (ESI): 190 [M+H]⁺, RT 0.77 min (HPLC, 1×50 mm, 2.5 μm, water/acetonitrile+0.05% formic acid, 35 μl/min).

### Synthesis of 2-isopropyl-5,7-dimethyl-3H-imidazo[4,5-b]pyridine (2d, R=iso-propyl)

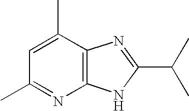

This compound was synthesized analogously to example 2c using iso-butyric acid.

MS (ESI): 190 [M+H]⁺, RT 0.77 min (HPLC, 1×50 mm, 2.5 μm, water/acetonitrile+0.05% formic acid, 35 μl/min).

### Synthesis of 2-cyclopropyl-5,7-dimethyl-3H-imidazo[4,5-b]pyridine (2e, R=cyclo-propyl)

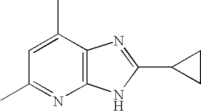

This compound was synthesized analogously to example 2c using cyclopropane carboxylic acid.

MS (ESI): 188 [M+H]⁺; RT 1.42 and 1.48 min (isomers) (HPLC, 2.1×50 mm, 2.5 μm, water/acetonitrile+0.05% formic acid).

### Synthesis of 2-butyl-5,7-dimethyl-3H-imidazo[4,5-b]pyridine (2f, R=n-butyl)

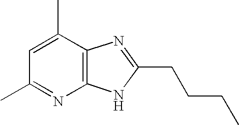

This compound was synthesized analogously to example 1b using n-pentanoic acid.

MS (ESI): 218 [M+H]⁺, RT 8.68 min (HPLC, 1×50 mm, 2.5 μm, water/3 mM NH₄Ac/acetonitrile+0.05% formic acid, 35 μl/min); ¹H-NMR (DMSO-d6, 400 MHz) δ (ppm): 12.42 (s, 1H), 6.84 (s, 1H), 2.8 (m, 2H), 2.46 (s, 6H), 1.67 (m, 2H), 1.58 (m, 1H), 0.49 (d, 6H).

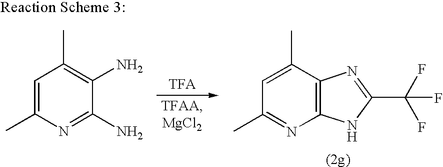

### Synthesis of 2-trifluoromethyl-5,7-dimethyl-3H-imidazo[4,5-b]pyridine (2g)

4,6-Dimethyl-pyridine-2,3-diamine (2 g, 12.26 mmol), trifluoroacetic acid (15 ml, 196 mmol) and MgCl₂ (1.17 g, 12.26 mmol) were dissolved in trifluoroacetic anhydride (15 ml) and the mixture was heated to 150° C. After stirring at this temperature for 48 h, the mixture was evaporated under reduced pressure and extracted with ethyl acetate and washed with water. The organic layer was evaporated under reduced pressure. The crude product was purified by flash chromatography (silical gel, ethyl acetate) to give an colorless oil,

MS (ESI): 216 [M+H]⁺; ¹H-NMR (DMSO-d6, 500 MHz) δ (ppm): 7.13 (s, 1H), 2.58 (s, 3H), 2.57 (s, 3H).

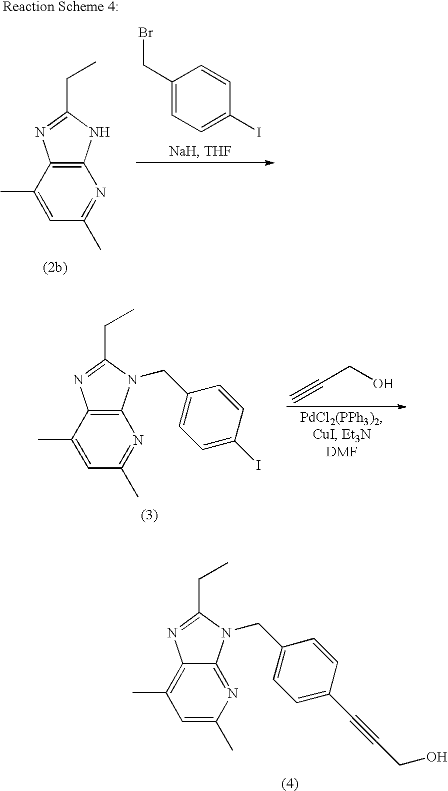

### Synthesis of 3-[4-(2-Ethyl-5,7-dimethyl-imidazo[4,5-b]pyridin-3-ylmethyl)-phenyl]-prop-yn-1-ol (4)

### (1) Step A: 2-Ethyl-3-(4-iodo-benzyl)-5,7-dimethyl-3H-imidazo[4,5-b]pyridine (3)

To a solution of 2-ethyl-5,7-dimethyl-3H-imidazo[4,5-b]pyridine (2b) (150 mg, 0.856 mmol) in 1.5 ml of THF was added NaH (45 mg, 1.88 mmol). After 10 minutes 4-iodobenzyl bromide (235 mg, 0.94 mmol) was added and the reaction mixture was stirred at rt for 12 h. The mixture was partitioned between CH₂Cl₂ and H₂O and the aqueous layer was re-extracted with CH₂Cl₂. The combined organic layers were dried over Na₂SO₄ and evaporated under reduced pressure. The crude product was purified by flash-chromatography (silica gel, MeOH/CH₂Cl₂, 0 . . . 5%).

MS (ESI): 344.1-346.1 [M+H]⁺, ¹H NMR (400 MHz, CHCl₃-d) δ (ppm): 1.32 (t, 3H) 2.61 (d, 6H) 2.77 (d, 2H) 5.40 (s, 2H) 6.90 (s, 1H) 7.01 (d, 2H) 7.41 (d, 2H).

### (2) Step B: 3-[4-(2-Ethyl-5,7-dimethyl-imidazo[4,5-b]pyridin-3-ylmethyl)-phenyl]-prop-yn-1-ol (4)

A solution of 2-ethyl-3-(4-iodo-benzyl)-5,7-dimethyl-3H-imidazo[4,5-b]pyridine (3) (94.5 mg, 0.242 mmol), propargyl alcohol (67.6 mg, 1.21 mmol), copper(I)iodide (2.3 mg, 0.012 mmol), PdCl₂(PPh₃)₂ (11.1 mg, 0.012 mmol) and triethylamine (335 μl, 2.4 mmol) in 3.5 ml of DMF was heated in a sealed tube to 100° C. for 1 hr. The reaction mixture was cooled to rt and partitioned between EtOAc/H₂O and extracted. The combined organic layers were dried over Na₂SO₄ and evaporated under reduced pressure. The crude product was purified by flash-chromatography (silica gel, hexanes/EtOAc, 0 . . . 100%).

MS (ESI): 320.4 [M+H]⁺, ¹H NMR (400 MHz, MeOD) δ (ppm): 1.69 (t, 3H) 3.03 (d, 6H) 3.29 (q, 2H) 5.31 (s, 2H) 5.98 (s, 2H) 7.45 (s, 1H) 7.53 (d, 2H) 7.80 (d, 2H).

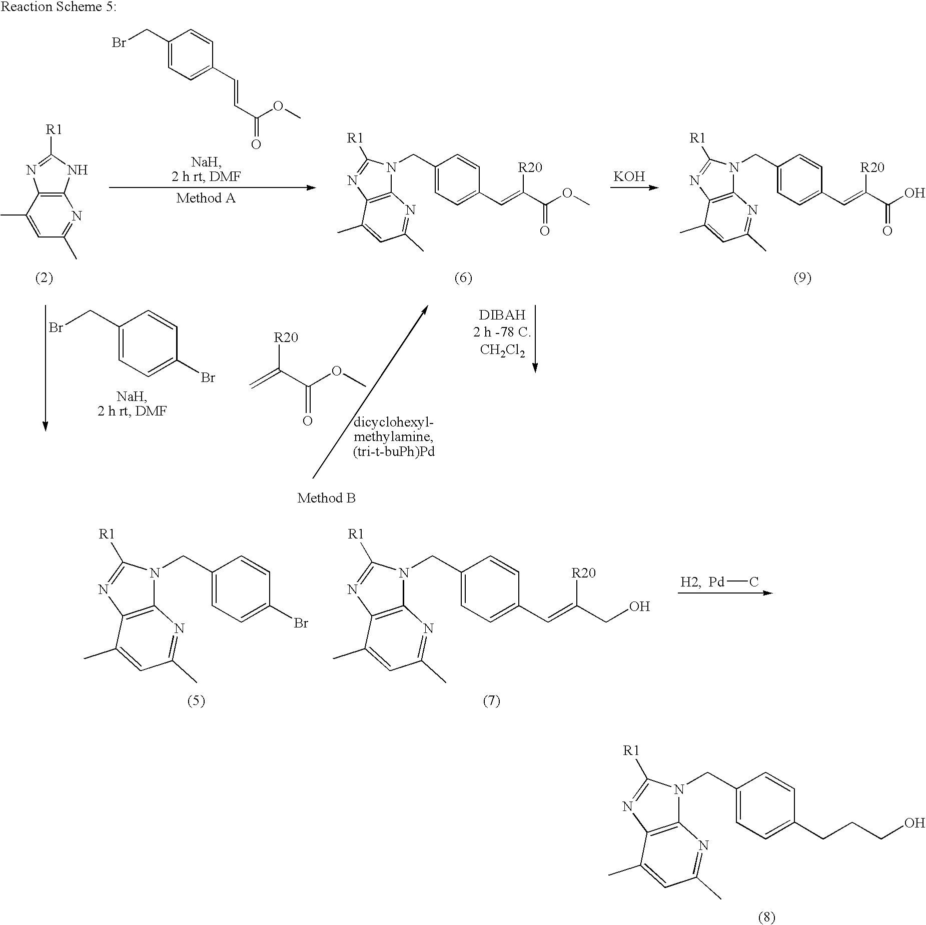

### Synthesis of (E)-3-[4-(2,5,7-Trimethyl-imidazo[4,5-b]pyridin-3-ylmethyl)-phenyl]-prop-2-en-1-ol (7a, R1=methyl, R20=H, Method A)

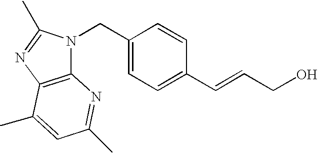

### (1) Step A: (E)-3-[4-(2,5,7-Trimethyl-imidazo[4,5-b]pyridin-3-ylmethyl)-phenyl]-acrylic acid methyl ester (6a)

2,5,7-Trimethyl-3H-imidazo[4,5-b]pyridine (2a) (1.0 g, 6.2 mmol) was dissolved in 62 ml of DMF and after addition of NaH (55% in mineral oil, 324 mg, 7.4 mmol) the mixture was stirred for 20 min. Methyl-p-bromomethyl cinnamate (1.6 g, 6.2 mmol) was added and the mixture was stirred for 2 h (TLC control) at rt. Then the mixture was evaporated (high vacuum). The residue was diluted with ethyl acetate, washed with water and brine, dried over Na₂SO₄ and evaporated. The crude product was purified by flash-chromatography (ethyl acetate/cyclohexanes (7:3), silicagel) to yield a colorless solid.

MS (ESI): 336 [M+H]⁺; ¹H-NMR (DMSO-d6, 400 MHz) δ (ppm): 7.63 (d, 2H), 7.56 (d, 1H), 7.14 (d, 2H), 6.91 (s, 1H), 6.58 (d, 1H), 5.44 (s, 2H), 3.69 (s, 3H), 2.49 (s, 3H), 2.48 (s, 3H), 2.43 (s, 3H).

### (2) Step B: (E)-3-[4-(2,5,7-Trimethyl-imidazo[4,5-b]pyridin-3-ylmethyl)-phenyl]-prop-2-en-1-ol (7a)

(E)-3-[4-(2,5,7-Trimethyl-imidazo[4,5-b]pyridin-3-ylmethyl)-phenyl]-acrylic acid methyl ester (6a) (780 mg, 2.3 mmol), was dissolved in 23 ml dichloromethane and cooled to −78° C. A 1.2M solution of DIBAH in dichloromethane (5.8 ml, 7 mmol) was added dropwise. The mixture was stirred for 3 h (TLC control) at −78° C. Then the mixture was quenched with water and evaporated. The residue was diluted with ethyl acetate, washed with water and NaCl-solution, dried over Na₂SO₄ and evaporated. The crude product was purified by flash-chromatography (ethyl acetate/hexanes (1:1), silicagel) to yield a colorless oil.

MS (ESI): 308 [M+H]⁺; ¹H-NMR (DMSO-d6, 400 MHz) δ (ppm): 7.34 (d, 2H), 7.08 (d, 21H), 6.92 (s, 1H), 6.47 (d, 1H), 6.34 (dt, 1H), 5.40 (s, 2H), 4.83 (t, 1H), 4.08 (t, 2H), 2.50 (s, 3H), 2.49 (s, 3H), 2.44 (s, 3H).

### Synthesis of (E)-3-[4-(2-Ethyl-5,7-dimethyl-imidazo[4,5-b]pyridin-3-ylmethyl)-phenyl]-prop-2-en-1-ol (7b, R1=ethyl, R20=H, Method A)

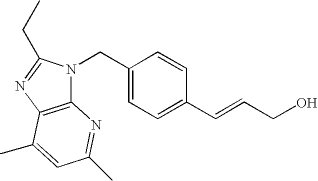

### (1) Step A: (E)-3-[4-(2-Ethyl-5,7-dimethyl-imidazo[4,5-b]pyridin-3-ylmethyl)-phenyl]-acrylic acid methyl ester (6b)

This compound was synthesized from 2b analogously to example 6a.

MS (ESI): 350 [M+H]⁺, ¹H-NMR (DMSO-d6, 400 MHz) δ (ppm): 7.67 (d, 2H), 7.63 (d, 1H), 7.15 (d, 2H), 6.96 (s, 1H), 6.6 (d, 1H), 5.49 (s, 2H), 3.72 (s, 3H), 2.77 (q, 2H), 2.52 (s, 3H), 2.50 (s, 3H), 1.23 (t, 3H).

### (2) Step B: (E)-3-[4-(2-Ethyl-5,7-dimethyl-imidazo[4,5-b]pyridin-3-ylmethyl)-phenyl]-prop-2-en-1-ol (7b)

This compound was synthesized from 6b analogously to example 7a.

MS (ESI): 322 [M+H]⁺, ¹H-NMR (DMSO-d6, 400 MHz) δ (ppm): 7.35 (d, 2H), 7.07 (d, 2H), 6.95 (s, 1H), 6.48 (d, 1H), 6.36 (dt, 1H), 5.43 (s, 2H), 4.85 (t, 1H), 4.10 (dt, 2H), 2.77 (q, 2H), 2.52 (s, 6H), 1.23 (t, 3H).

### Synthesis of 3-[4-(2-Ethyl-5,7-dimethyl-imidazo[4,5-b]pyridin-3-ylmethyl)-phenyl]-propan-1-ol (8b, R1=ethyl)

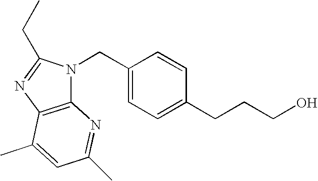

Pd—C (3.24 g, 3.05 mmol) was suspended in 350 ml ethanol at rt. After addition of a solution of (E)-3-[4-(2-ethyl-5,7-dimethyl-imidazo[4,5-b]pyridin-3-ylmethyl)-phenyl]-prop-2-en-1-ol (7b) (9.8 g, 30.5 mmol) in 100 ml of ethanol and ammonium formiate (7.69 g, 122 mmol) the mixture was stirred for 1 h at rt. Then the mixture was filtered through celite and evaporated under reduced pressure. The crude product was used in the next step without further purification.

MS (ESI): 324 [M+H]⁺, ¹H-NMR (DMSO-d6, 500 MHz) δ (ppm): 7.12 (d, 2H), 7.01 (d, 2H), 6.92 (s, 1H), 6.39 (s, 2H), 3.36 (t, 2H), 2.74 (q, 2H), 2.55 (t, 2H), 2.49 (s, 6H), 1.64 (m, 2H), 1.20 (t, 3H).

### Synthesis of (E)-3-[4-(2-Ethyl-5,7-dimethyl-imidazo[4,5-b]pyridin-3-ylmethyl)-phenyl]-acrylic acid (9b, R1=ethyl, R20=H)

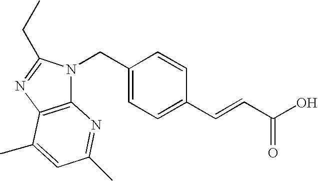

(E)-3-[4-(2-Ethyl-5,7-dimethyl-imidazo[4,5-b]pyridin-3-ylmethyl)-phenyl]-acrylic acid methyl ester (6b) (795 mg, 2.3 mmol), was dissolved in 9 ml of a mixture of THF, ethanol and water (1:1:1) and after addition of KOH pellets (640 mg, 11.4 mmol) the mixture was stirred for 2 h (TLC control) at 85° C. Then the organic solvent was removed under reduced pressure. The mixture was treated with 2M HCl until pH1 was reached. The free acid was then extracted with ethyl acetate several times, dried over Na₂SO₄ and evaporated. The crude product was purified by recrystallization from ethyl acetate to give a white solid.

MS (ESI): 336 [M+H]⁺, ¹H-NMR (DMSO-d6, 500 MHz) δ (ppm): 12.40 (br s, 1H), 7.64 (d, 2H), 7.53 (d, 1H), 7.20 (d, 2H), 7.06 (s, 1H), 6.51 (d, 1H), 5.54 (s, 2H), 2.86 (m, 2H), 2.55 (s, 3H), 2.53 (s, 3H), 1.25 (t, 3H).

### Synthesis of (E)-3-[4-(5,7-Dimethyl-2-propyl-imidazo[4,5-b]pyridin-3-ylmethyl)-phenyl]-prop-2en-1-ol (7c, R1=propyl, R20=H, Method B)

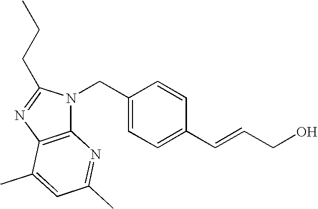

### (1) Step A: 3-(4-Bromo-benzyl)-5,7-dimethyl-2-propyl-3H-imidazo[4,5-b]pyridine (5c)

5,7-Dimethyl-2-propyl-3H-imidazo[4,5-b]pyridine (2c) (520 mg, 2.75 mmol) was dissolved in 27 ml of DMF, followed by addition of NaH (60% in mineral oil, 132 mg, 3.3 mmol). The mixture was stirred for 20 min at rt. Then 4-bromobenzylbromide (687 mg, 2.75 mmol) was added and the mixture was stirred for 2 h at rt (TLC control). The mixture was evaporated under reduced pressure, the residue was diluted with ethyl acetate, washed with brine and dried over Na₂SO₄. Evaporation gave a yellow oil. The crude product was purified by flash-chromatography (ethyl acetate/cyclohexanes 9:1, silicagel) giving an colorless solid.

MS (ESI): 358, 360 [M+H]⁺, ¹H-NMR (DMSO-d6, 500 MHz) δ (ppm): 7.37 (d, 2H), 6.92 (d, 2H), 6.79 (s, 1H), 5.27 (s, 2H), 2.56 (t, 2H), 2.34 (s, 3H), 2.33 (s, 3H), 1.51 (h, 1H), 0.74 (t, 3H).

### (2) Step B: (E)-3-[4-(5,7-Dimethyl-2-propyl-imidazo[4,5-b]pyridin-3-ylmethyl)-phenyl]-acrylic acid methyl ester (6c)

3-(4-Bromo-benzyl)-5,7-dimethyl-2-propyl-3H-imidazo[4,5-b]pyridine (5c) (525 mg, 1.47 mmol) was dissolved in 15 ml of dioxane and after addition of methyl acrylate (264 ul, 2.93 mmol), dicyclohexyl-methylamine (622 ul ml, 2.93 mmol) and Pd(PtBu₃)₂ (15 mg, 0.03 mmol) the mixture was heated for 5 min at 130° C. in a microwave oven. Then the mixture was evaporated under reduced pressure. The residue was diluted with ethyl acetate, washed with sat. NaHCO₃— and NaCl-solution, and dried over Na₂SO₄. Evaporation gave a yellow solid. The crude product was purified by recrystallization from diethyl ether to give a colorless solid.

MS (ESI): 364 [M+H]⁺, ¹H-NMR (DMSO-d6, 500 MHz) δ (ppm): 7.66 (d, 2H), 7.60 (d, 1H), 7.13 (d, 2H), 6.95 (s, 1H), 6.60 (d, 1H), 5.48 (s, 2H), 3.71 (s, 3H), 2.73 (t, 2H), 2.51 (s, 3H), 2.49 (s, 3H), 1.68 (m, 2H), 0.90 (t, 3H).

### (3) Step C: (E)-3-[4-(5,7-Dimethyl-2-propyl-imidazo[4,5-b]pyridin-3-ylmethyl)-phenyl]-prop-2-en-1-ol (7c)

(E)-3-[4-(5,7-dimethyl-2-propyl-imidazo[4,5-b]pyridin-3-ylmethyl)-phenyl]-acrylic acid methyl ester (6c) (350 mg, 0.96 mmol) was dissolved in 10 ml of CH₂Cl₂ and cooled to −78° C. After addition of a 1.2M solution of DIBAH in CH₂Cl₂ (2.4 ml, 2.89 mmol) the mixture was stirred for 1 h (TLC control) at −78° C. Then the reaction mixture was quenched by addition of water and evaporated under reduced pressure. The residue was diluted with ethyl acetate, washed with water and brine, dried over Na₂SO₄ and evaporated. The crude product was purified by flash-chromatography (ethyl acetate/cyclohexanes 1:9, silicagel) to give a colorless solid.

MS (ESI): 336 [M+H]⁺, ¹H-NMR (DMSO-d6, 500 MHz) δ (ppm): 7.37 (d, 2H), 7.07 (d, 2H), 6.96 (s, 1H), 6.51 (d, 1H), 6.35 (dt, 1H), 5.44 (s, 2H), 4.85 (t, 1H), 4.10 (m, 2H), 2.74 (t, 2H), 2.51 (s, 6H), 1.69 (m, 2H), 0.90 (t, 3H).

### Synthesis of (E)-3-[4-(2-Isopropyl-5,7-dimethyl-imidazo[4,5-b]pyridin-3-ylmethyl)-phenyl]-prop-2-en-1-ol (7d, i-propyl, R20=H, Method B)

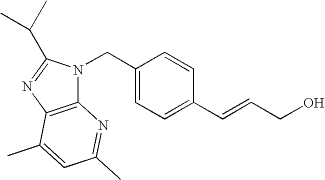

### (1) Step A: 3-(4-Bromo-benzyl)-2-isopropyl-5,7-dimethyl-3H-imidazo[4,5-b]pyridine (5d)

This compound was synthesized from 2d analogously to example 5c.

MS (ESI): 358, 360 [M+H]⁺, ¹H-NMR (DMSO-d6, 500 MHz) δ (ppm): 7.53 (d, 2H), 7.06 (d, 2H), 6.96 (s, 1H), 5.48 (s, 2H), 3.17 (h, 1H), 2.50 (d, 6H), 1.20 (d, 6H).

### (2) Step B: (E)-3-[4-(2-Isopropyl-5,7-dimethyl-imidazo[4,5-b]pyridin-3-ylmethyl)-phenyl]-acrylic acid methyl ester (6d)

This compound was synthesized from 5d analogously to example 6c.

MS (ESI): 364 [M+H]⁺, ¹H-NMR (DMSO-d6, 500 MHz) δ (ppm): 7.68 (d, 2H), 7.63 (d, 1H), 7.14 (d, 2H), 6.97 (s, 1H), 6.61 (d, 1H), 5.53 (s, 2H), 3.72 (s, 3H), 3.18 (h, 1H), 2.53 (s, 3H), 2.50 (s, 3H), 1.21 (d, 6H).

### (3) Step C: (E)-3-[4-(2-Isopropyl-5,7-dimethyl-imidazo[4,5-b]pyridin-3-ylmethyl)-phenyl]-prop-2-en-1-ol (7d)

This compound was synthesized from 6d analogously to example 7c.

MS (ESI): 336 [M+H]⁺, ¹H-NMR (DMSO-d6, 500 MHz) δ (ppm): 7.35 (d, 2H), 7.05 (d, 2H), 6.95 (s, 1H), 6.48 (d, 1H), 6.35 (dt, 1H), 5.47 (s, 2H), 4.84 (t, 1H), 4.09 (dd, 2H), 3.17 (h, 1H), 2.51 (s, 6H), 1.18 (d, 6H).

### Synthesis of (E)-3-[4-(2-Cyclopropyl-5,7-dimethyl-imidazo[4,5-b]pyridin-3-ylmethyl)-phenyl]-prop-2-en-1-ol (7e, R1=cyclopropyl, R20=H, Method A)

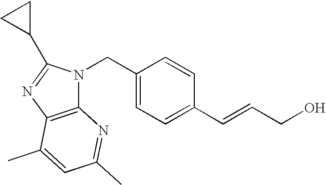

### (1) Step A: (E)-3-[4-(2-Cyclopropyl-5,7-dimethyl-imidazo[4,5-b]pyridin-3-lmethyl)-phenyl]-acrylic acid methyl ester (6e)

This compound was synthesized from 2e analogously to example 6a. MS (ESI): 362 [M+H]⁺.

### (2) Step B: (E)-3-[4-(2-Cyclopropyl-5,7-dimethyl-imidazo[4,5-b]pyridin-3-ylmethyl)-phenyl]-prop-2-en-1-ol (7e)

This compound was synthesized from 6e analogously to example 7a.

MS (ESI): 334 [M+H]⁺, RT 0.91 min (HPLC, 1×50 mm, 2.5 μm, water/acetonitrile+0.05% formic acid, 35 μl/min).

### Synthesis of (E)-3-[4-(2-Butyl-5,7-dimethyl-imidazo[4,5-b]pyridin-3-ylmethyl)-phenyl]-prop-2-en-1-ol (7f, R1=n-butyl, R20=H, Method B)

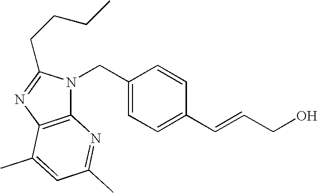

### (1) Step A: 3-(4-Bromo-benzyl)-2-butyl-5,7-dimethyl-3H-imidazo[4,5-b]pyridine (5f)

This compound was synthesized analogously to 5c starting from compound 2f.

MS (ESI): 372, 374 [M+H]⁺, ¹H-NMR (DMSO-d6, 400 MHz) δ (ppm): 7.53 (d, 2H), 7.07 (d, 2H), 6.96 (s, 1H), 5.44 (s, 2H), 2.75 (t, 2H), 2.52 (s, 3H), 2.50 (s, 3H), 1.75 (m, 2H), 1.34 (m, 2H), 0.84 (t, 3H).

### (2) Step B: (E)-3-[4-(2-Butyl-5,7-dimethyl-imidazo[4,5-b]pyridin-3-ylmethyl)-phenyl]-acrylic acid methyl ester (6f)

This compound was synthesized analogously to 6c starting from compound 5f.

MS (ESI): 378 [M+H]⁺, ¹H-NMR (DMSO-d6, 400 MHz) δ (ppm): 7.68 (d, 2H), 7.62 (d, 1H), 7.14 (d, 2H), 6.96 (s, 1H), 6.60 (d, 1H), 5.49 (s, 2H), 3.72 (s, 3H), 2.75 (t, 2H), 2.52 (s, 3H), 2.50 (s, 3H), 1.63 (m, 2H), 1.32 (m, 2H), 0.84 (t, 3H).

### (3) Step C: (E)-3-[4-(2-Butyl-5,7-dimethyl-imidazo[4,5-b]pyridin-3-ylmethyl)-phenyl]-prop-2-en-1-ol (7f)

This compound was synthesized analogously to 7c starting from compound 6f.

MS (ESI): 350 [M+H]⁺, ¹H-NMR (DMSO-d6, 400 MHz) δ (ppm): 7.37 (d, 2H), 7.08 (d, 2H), 6.96 (s, 1H), 6.51 (d, 1H), 6.35 (dt, 1H), 5.45 (s, 2H), 4.87 (t, 1H), 4.11 (dd, 2H), 2.76 (t, 2H), 1.63 (m, 2H), 1.32 (m, 2H), 0.84 (t, 3H).

### Synthesis of (E)-3-[4-(2-Trifluoromethyl-5,7-dimethyl-imidazo[4,5-b]pyridin-3-ylmethyl)-phenyl]-prop-2-en-1-ol (7g, R1=CF₃, R20=H, Method A)

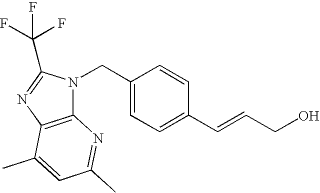

### (1) Step A: (E)-3-[4-(5,7-Dimethyl-2-trifluoromethyl-imidazo[4,5-b]pyridin-3-ylmethyl)-phenyl]-acrylic acid methyl ester (6g)

This compound was synthesized from compound 2g analogously to 6a.

MS (ESI): 390 [M+H]⁺, ¹H-NMR (DMSO-d6, 500 MHz) δ (ppm): 7.67 (d, 2H), 7.62 (d, 1H), 7.24 (s, 1H), 7.14 (d, 2H), 6.60 (d, 1H), 5.67 (s, 2H), 3.71 (s, 3H), 2.60 (s, 3H), 2.58 (s, 3H).

### (2) Step B: (E)-3-[4-(5,7-Dimethyl-2-trifluoromethyl-imidazo[4,5-b]pyridin-3-ylmethyl)-phenyl]-prop-2-en-1-ol (7d)

This compound was synthesized analogously to 7a starting from compound 6g.

MS (ESI): 362 [M+H]⁺, ¹H-NMR (DMSO-d6, 500 MHz) δ (ppm): 7.35 (d, 2H), 7.23 (s, 1H), 7.07 (d, 2H), 6.49 (d, 1H), 6.35 (dt, 1H), 5.61 (s, 2H), 4.84 (t, 1H), 4.08 (m, 2H), 2.59 (s, 3H), 2.58 (s, 3H).

### Synthesis of (Z)-3-[4-(2-Ethyl-5,7-dimethyl-imidazo[4,5-b]pyridin-3-ylmethyl)-phenyl]-2-fluoro-prop-2-en-1-ol (7h, R1=ethyl, R20=F, Method B)

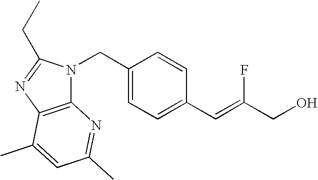

### (1) Step A: 3-(4-Bromo-benzyl)-2-ethyl-5,7-dimethyl-3H-imidazo[4,5-b]pyridine (5h)

This compound was synthesized analogously to 5c starting from compound 2b.

MS (ESI): 344 and 346 [M+H]⁺; ¹H-NMR (DMSO-d6, 500 MHz) δ (ppm): 7.51 (d, 2H), 7.05 (d, 2H), 6.94 (s, 1H), 5.42 (s, 2H), 2.75 (q, 2H), 2.49 (s, 6H), 1.21 (t, 3H).

### (2) Step B: (Z)-3-[4-(2-Ethyl-5,7-dimethyl-imidazo[4,5-b]pyridin-3-ylmethyl)-phenyl]-2-fluoro-acrylic acid methyl ester (6h)

This compound was synthesized analogously to 6c starting from compound 5h.

MS (ESI): 368 [M+H]⁺, ¹H-NMR (DMSO-d6, 500 MHz) δ (ppm): 7.64 (d, 2H), 7.18 (d, 2H), 7.05 (d, 1H), 6.94 (s, 1H), 5.48 (s, 2H), 3.81 (s, 3H), 2.75 (q, 2H), 2.49 (s, 6H), 1.21 (t, 3H).

### (3) Step C: (Z)-3-[4-(2-Ethyl-5,7-dimethyl-imidazo[4,5-b]pyridin-3-ylmethyl)-phenyl]-2-fluoro-prop-2-en-1-ol (10d)

This compound was synthesized analogously to 7c starting from compound 6h.

MS (ESI): 340 [M+H]⁺, ¹H-NMR (DMSO-d6, 500 MHz) δ (ppm): 7.41 (d, 2H), 7.10 (d, 2H), 6.94 (s, 1H), 5.85 (d, 1H), 5.43 (s, 2H), 5.40 (t, 1H), 4.08 (dd, 2H), 2.77 (q, 2H), 2.50 (s, 6H), 1.21 (t, 3H).

### Synthesis of 4-(2-Ethyl-5,7-dimethyl-imidazo[4,5-b]pyridin-3-ylmethyl)-benzoic acid (11)

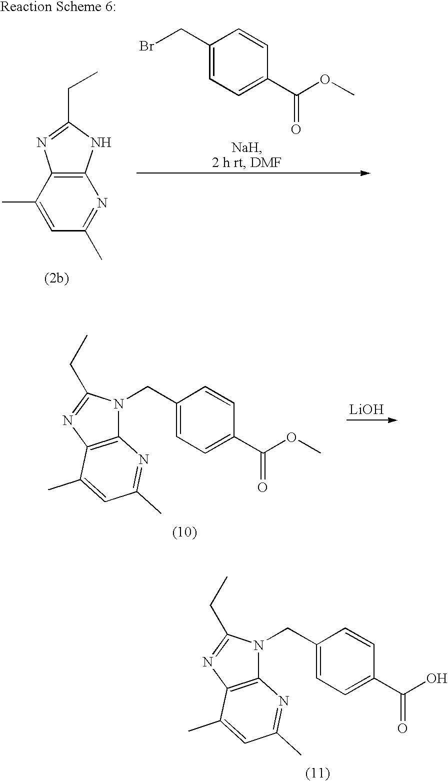

### (1) Step A: 4-(2-Ethyl-5,7-dimethyl-imidazo[4,5-b]pyridin-3-ylmethyl)-benzoic acid methyl ester (10)

2-Ethyl-5,7-dimethyl-3H-imidazo[4,5-b]pyridine (2b) (1 g, 5.71 mmol) was dissolved in 57 ml of DMF and after addition of 4-bromomethyl-benzoic acid methyl ester (1.3 g, 5.71 mmol) and NaH (60% in mineral oil, 274 mg, 6.85 mmol) the mixture was stirred for 2 h at rt. The solvent was evaporated and the residue was diluted with ethyl acetate, washed with water and brine, dried over Na₂SO₄ and evaporated under reduced pressure. The crude product was purified by recrystallization from ethyl acetate to give a colorless solid.

MS (ESI): 324 [M+H]⁺, ¹H-NMR (DMSO-d6, 500 MHz) δ (ppm): 7.87 (d, 2H), 7.20 (d, 2H), 6.93 (s, 1H), 5.52 (s, 2H), 3.81 (s, 3H), 2.72 (q, 2H), 2.50 (s, 3H), 2.47 (s, 3H), 1.20 (t, 3H).

### (2) Step B: 4-(2-Ethyl-5,7-dimethyl-imidazo[4,5-b]pyridin-3-ylmethyl)-benzoic acid (11)

4-(2-Ethyl-5,7-dimethyl-imidazo[4,5-b]pyridin-3-ylmethyl)-benzoic acid methyl ester (10) (680 mg, 2.1 mmol) was dissolved in 10 ml of THF and after addition of a solution of LiOH (103 mg, 4.21 mmol) in 10 ml of water the mixture was refluxed for 4 h. Then the organic solvent were removed under reduced pressure. The residue was cooled to 0° C. and treated with 2M HCl. The crude product was filtered off and dried under high vacuum. The product was used in the next step without further purification.

MS (ESI): 310 [M+H]⁺, ¹H-NMR (DMSO-d6, 400 MHz) δ (ppm): 12.96 (br s, 1 H), 7.88 (d, 2H), 7.32 (d, 2H), 7.22 (s, 1H), 5.68 (s, 2H), 3.01 (q, 2H), 2.60 (s, 3H), 2.55 (s, 3H), 1.26 (t, 3H).

### Synthesis of 2-[4-(2-Ethyl-5,7-dimethyl-imidazo[4,5-b]pyridin-3-ylmethyl)-phenyl]-ethanol (13)

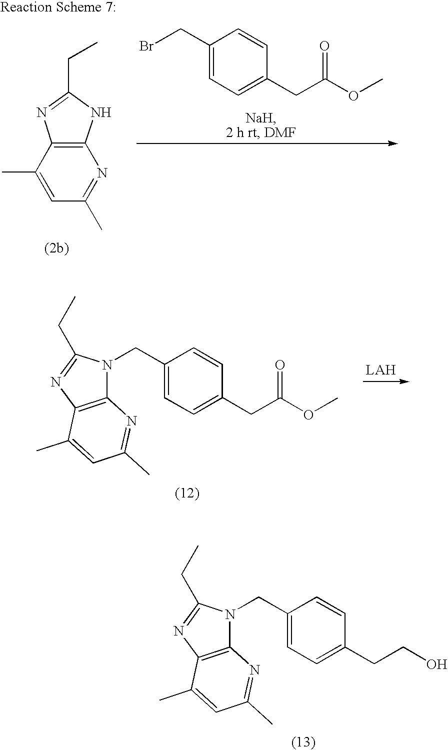

### (1) Step A: [4-(2-Ethyl-5,7-dimethyl-imidazo[4,5-b]pyridin-3-ylmethyl)-phenyl]-acetic acid methyl ester (12)

2-Ethyl-5,7-dimethyl-3H-imidazo[4,5-b]pyridine (2b) (1 g, 5.71 mmol) was dissolved in 10 ml of DMF and after addition of NaH (60% in mineral oil, 0.228 g, 5.71 mmol) the mixture was stirred for 20 min at rt. Then methyl 4-(bromomethyl)phenylacetate (1.387 g, 5.71 mmol) was added and the mixture was stirred for 4 h at rt. The solvent was evaporated under reduced pressure and the residue was diluted with ethyl acetate, washed with water and NaCl-solution, dried over Na₂SO₄ and evaporated. The crude product was purified by chromatography on silica (ethyl acetate/cyclohexanes 1:2).

MS (ESI): 338 [M+H]⁺, ¹H-NMR (DMSO-d6, 400 MHz) δ (ppm): 7.17 (d, 2H), 7.03 (d, 2H), 6.91 (s, 1H), 5.40 (s, 2H), 3.61 (s, 2H), 3.56 (s, 3H), 2.75 (q, 2H), 2.49 (s, 3H), 2.48 (s, 3H), 1.21 (t, 3H).

### (2) Step B: 2-[4-(2-Ethyl-5,7-dimethyl-imidazo[4,5-b]pyridin-3-ylmethyl)-phenyl]-ethanol (13)

A 1M solution of LiAlH₄ in THF (2.55 ml, 2.55 mmol) was diluted with 2 ml of THF and cooled to 0° C. [4-(2-Ethyl-5,7-dimethyl-imidazo[4,5-b]pyridin-3-ylmethyl)-phenyl]acetic acid methyl ester (12) (430 mg, 1.27 mmol) was dissolved in 3 ml of THF and added dropwise. The mixture was stirred for 2 h (TLC control) at 0° C. Then the mixture was cooled to −15° C. and 1M NaOH was added dropwise (strongly exothermic, gas formation). Then the mixture was filtrated through celite, the filter cake was washed with THF and the combined filtrates were evaporated. The residue was purified by chromatography on silica (ethyl acetate/cyclohexanes 1:1).

MS (ESI): 310 [M+H]⁺, ¹H-NMR (DMSO-d6, 500 MHz) δ (ppm): 7.14 (d, 2H), 7.01 (d, 2H), 6.93 (s, 1H), 5.39 (s, 2H), 4.85 (m, 1H), 3.54 (m, 2H), 2.76 (q, 2H), 2.65 (t, 2H), 2.49 (s, 6H), 1.22 (t, 3H).

### Synthesis of 4-(2-Ethyl-5,7-dimethyl-imidazo[4,5-b]pyridin-3-ylmethyl)-phenylamine (14)

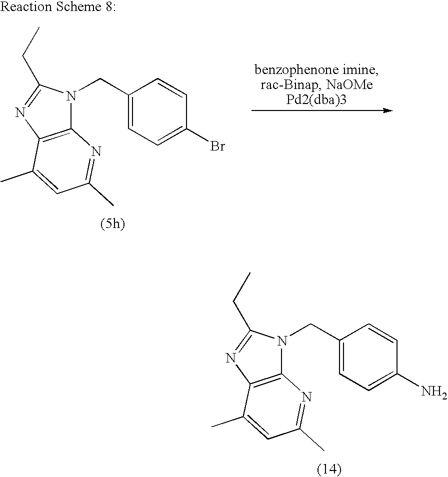

3-(4-Bromo-benzyl)-2-ethyl-5,7-dimethyl-3H-imidazo[4,5-b]pyridine (5h, 650 mg, 1.888 mmol), benzophenoneimine (411 mg, 2.266 mmol), CH₃ONa (153 mg, 2.830 mmol), rac-BINAP (88 mg, 0.142 mmol) and Pd₂(dba)₃ (43.2 mg, 0.047 mmol) were suspended in toluene (6 ml) under nitrogen. The mixture was stirred for 4 h at 100° C. Then the mixture was cooled to rt and 2M HCl (20 ml) was added followed by stirring for 30 min. The mixture was washed with EtOAc and the aqueous layer was slowly basified with Na₂CO₃ solid until pH>8, then again extracted with EtOAc/brine. The organic layer was dried over Na₂SO₄ and evaporated. The crude product was purified chromatography on silica (0% to 2% MeOH in CH₂Cl₂) to give the title compound as a colorless oil.

LC-MS (APCI): 281 [M+H]+, t_{R}: 0.59 min. (Symmetry C8, 2×50 mm 3 um, 1 ml/min, H₂O (5% acetonitrile and 0.1% TFA)/acetonitrile (0.1% TFA), Gradient 0-2 min: 5% to 95%, 2-3 min: 95%, 3-3.3 min: 95% to 5%)

### Synthesis of 4-(2-Ethyl-5,7-dimethyl-imidazo[4,5-b]pyridin-3-ylmethyl)-phenol (16)

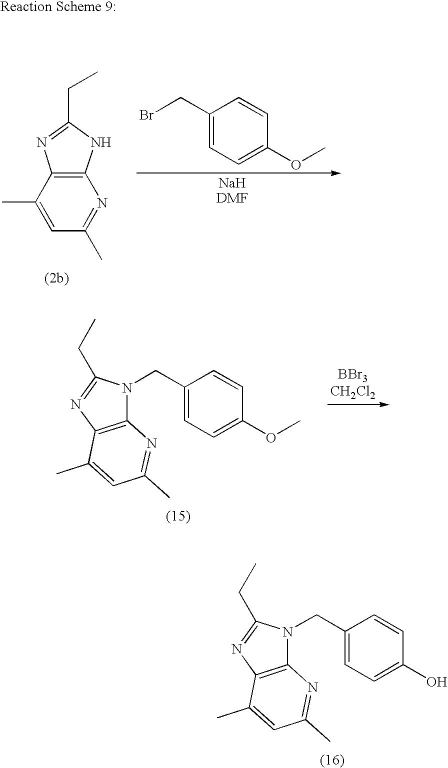

### (1) Step A: 2-Ethyl-3-(4-methoxy-benzyl)-5,7-dimethyl-3H-imidazo[4,5-b]pyridine (15)

2-Ethyl-5,7-dimethyl-3H-imidazo[4,5-b]pyridine (2b) (1 g, 5.7 mmol) was dissolved in 20 ml of DMF and NaH (60% in mineral oil, 500 mg, 11.4 mmol) was added. After 20 min stirring at rt, 1-bromomethyl-4-methoxy-benzene (0.82 ml, 5.7 mmol) was added slowly and the mixture was stirred for 4 h at rt (TLC control). Then the mixture was evaporated under reduced pressure. The residue was diluted with ethyl acetate, washed with water and NaCl-solution, dried over Na₂SO₄ and evaporated. The crude product was purified by flash-chromatography (ethyl acetate/cyclohexanes (1:1), silicagel) giving a white solid.

MS (ESI): 296 [M+H]⁺, ¹H-NMR (DMSO-d6, 500 MHz) δ (ppm): 7:06 (d, 2H), 6.91 (s, 1H), 6.86 (d, 2H), 5.34 (s, 2H), 3.68 (s, 3H), 2.74 (q, 2H), 2.49 (s, 3H), 2.47 (s, 3H), 1.19 (t, 3H).

### (2) Step B: 4-(2-Ethyl-5,7-dimethyl-imidazo[4,5-b]pyridin-3-ylmethyl)-phenol (16)

2-Ethyl-3-(4-methoxy-benzyl)-5,7-dimethyl-3H-imidazo[4,5-b]pyridine (15) (1.1 g, 3.7 mmol) was dissolved in 10 ml of CH₂Cl₂ and cooled to 0° C. A 1M solution of BBr₃ in methylene chloride (7.4 ml, 7.4 mmol) was added within 25 min. The reaction was allowed to stir for 2 h at rt. Then the mixture was poured on ice/5%-NaHCO₃-solution. The organic layer was separated, washed with water and saturated NaCl-solution and dried over Na₂SO₄. Evaporation gives a yellow solid, which was used in the next step without further purification.

MS (ESI): 282 [M+H]⁺, ¹H-NMR (DMSO-d6, 500 MHz) δ (ppm): 6.95 (d, 2H), 6.90 (s, 1H), 6.86 (d, 2H), 5.28 (s, 2H), 2.72 (q, 2H), 2.48 (s, 3H), 2.46 (s, 3H), 1.17 (t, 3H).

### Synthesis of (E)-4-[4-(2-Ethyl-5,7-dimethyl-imidazo[4,5-b]pyridin-3-ylmethyl)-phenyl]-but-3-en-1-ol (18)

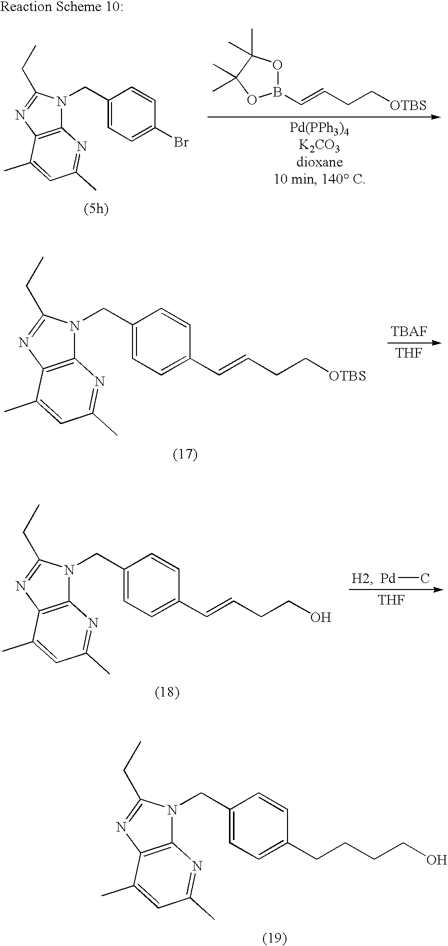

### (1) Step A: 3-{4-[(E)-4-(tert-Butyl-dimethyl-silanoxy)-but-1-enyl]-benzyl}-2-ethyl-5,7-dimethyl-3H-imidazo[4,5-b]pyridine (17)

3-(4-Bromo-benzyl)-2-ethyl-5,7-dimethyl-3H-imidazo[4,5-b]pyridine (5h, 410 mg, 1.19 mmol), (E)-4-(tert-butyldimethylsiloxy)-1-buten-1ylboronic acid pinacol ester (372 mg, 1.19 mmol) and tetrakis-(triphenylphoshine)-palladium(0) (69 mg, 0.06 mmol) were dissolved in a mixture of dioxane (8 ml) and 2M aq. potassium carbonate (1.5 ml) and heated to 140° C. for 30 min in a microwave oven. The reaction mixture was cooled to rt, diluted with ethyl acetate and washed with aqueous sodium bicarbonate and brine. The organic layer was dried over Na₂SO₄, filtered and evaporated. The residue was purified by column chromatography on silica (cyclohexane/ethyl acetate 1:1) to yield a colorless oil.

MS (ESI): 450 [M+H]⁺, ¹H-NMR (DMSO-d6, 500 MHz) δ (ppm): 7.29 (d, 2H), 7.03 (d, 2H), 6.93 (s, 1H), 6.38 (d, 1H), 6.20 (dt, 1H), 5.40 (s, 2H), 3.65 (t, 2H), 2.74 (q, 2H), 2.49 (2s, 6H), 2.32 (m, 2H), 1.18 (t, 3H), 0.83 (s, 9H), 0.0 (s, 6H).

### (2) Step B: (E)-4-[4-(2-Ethyl-5,7-dimethyl-3H-imidazo[4,5-b]pyridin3-ylmethyl)-phenyl]-but-3-en-1-ol (18)

3-{4-[(E)-4-(tert-Butyl-dimethyl-silanoxy)-but-1-enyl]-benzyl}-2-ethyl-5,7-dimethyl-3H-imidazo[4,5b]pyridine (17) (400 mg, 0.89 mmol) was dissolved in THF (5 ml) and a 1.6M solution of TBAF in THF (1.4 ml) was added. After stirring at rt for 2 h the reaction mixture was evaporated and the residue was purified by column chromatography on silica (gradient from cyclohexane/ethyl acetate 1:1 to 100% ethyl acetate) to yield a colorless solid.

MS (ESI): 336 [M+H]⁺, ¹H-NMR (DMSO-d6, 500 MHz) δ (ppm): 7.30 (d, 2H), 7.04 (d, 2H), 6.93 (s, 1H), 6.38 (d, 1H), 6.20 (dt, 1H), 5.40 (s, 2H), 4.55 (t, 1H), 3.47 (m, 2H), 2.73 (q, 2H), 2.49 (2s, 6H), 2.30 (m, 2H), 1.19 (t, 3H).

### (3) Step C: 4-[4-(2-Ethyl-5,7-dimethyl-imidazo[4,5-b]pyridin3-ylmethyl)-phenyl]-butan-1-ol (19)

A mixture of 10% palladium on charcoal (63 mg) an (E)-4-[4-(2-ethyl-5,7-dimethyl-3H-imidazo[4,5-b]pyridin3-ylmethyl)-phenyl]-but-3-en-1-ol (18) (200 mg, 0.6 mmol) in THF (5 ml) was stirred under an atmosphere of hydrogen at rt for 2 hours. The catalyst was removed by filtration through a plug of celite and the solvent was removed to give a colorless oil which was used in the next steps without further purification.

MS (ESI): 338 [M+H]⁺, ¹H-NMR (DMSO-d6, 500 MHz) δ (ppm): 7.11 (d, 2H), 7.01 (d, 2H), 6.93 (s, 1H), 5.39 (s, 2H), 3.36 (m, 2H), 2.74 (q, 2H), 2.52 (m, 2H), 2.49 (2s, 6H), 2.30 (m, 2H), 1.53 (m, 2H), 1.38 (m, 2H), 1.20 (t, 3H).

Synthesis of the phenyl-propynyl-amines

The phenyl-propynyl-amines were Generally Prepared by a Synthesis as Lined Out in Reaction Scheme 10.

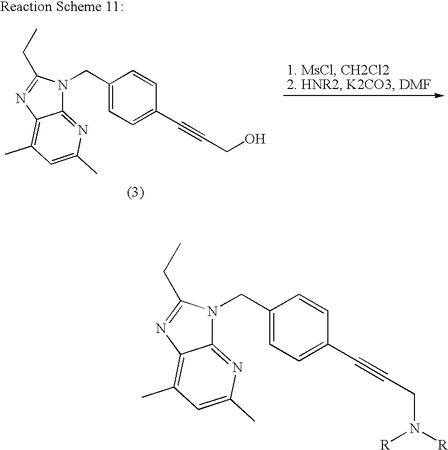

### EXAMPLE 1

### 2-Ethyl-3-{4-[3-(4-isopropyl-piperazin-1-yl)-prop-1-ynyl]-benzyl}-5,7-dimethyl-3H-imidazo[4,5-b]pyridine

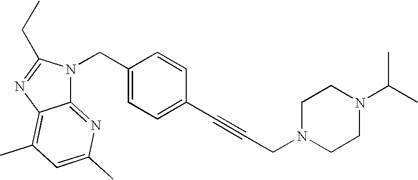

To a solution of 3-[4-(2-Ethyl-5,7-dimethyl-imidazo[4,5-b]pyridin-3-ylmethyl)-phenyl]-prop-yn-1-ol (3) (22 mg, 0.069 mmol) in CH₂Cl₂ was added mesylchloride (16.2 μl, 0.206 mmol) followed by diisopropylethylamine (36.1 μl, 0.206 mmol). After 5 min 4-isopropyl piperazine (51.7 μl, 0.344 mmol) was added followed by THF and the mixture was stirred at 80° C. for 1 h. Then the solvent was removed under reduced pressure. The crude product was dissolved in DMSO and purified by preparative reverse phase HPLC (gradient 0 . . . 60% MeCN/H₂O; 0.1% TFA).

MS (ESI): 430.1 [M+H]⁺, ¹H-NMR (MeOD, 400 MHz) δ (ppm): 1.32-1.41 (m, 9H) 2.67 (s, 6H) 2.87 (br. s., 4H) 3.21 (q, 2H) 3.47 (br. s., 4H) 3.5 (q, 1H) 3.73 (s, 2H) 5.75 (s, 2H) 7.38 (s, 1H) 7.32 (d, J=8.21 Hz, 2H) 7.44 (d, J=8.34 Hz, 2H).

### EXAMPLE 2

### 1′-{3-[4-(2-Ethyl-5,7-dimethyl-imidazo[4,5-b]pyridin-3-ylmethyl)-phenyl]-prop-2-ynyl}-[1,4′]bipiperidine

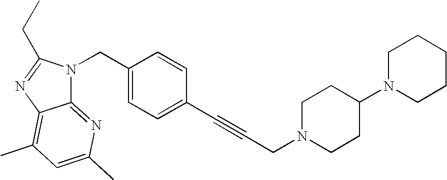

This compound was synthesized analogously to example 1 from 3-[4-(2-ethyl-5,7-dimethyl-imidazo[4,5-b]pyridin-3-ylmethyl)-phenyl]-prop-yn-1-ol (3) and 4-piperidinopiperidine.

MS (ESI): 470 [M+H]⁺, ¹H-NMR (MeOD, 400 MHz) δ (ppm): 1.35 (t, 3H) 1.85 (br. s., 4H) 2.03 (s, 5H) 2.39 (d, 2H) 2.67 (d, 6H) 3.13 (m, 6H) 3.50 (br. s., 4H) 3.75 (s, 2H) 4.21 (s, 2H) 5.74 (s, 2H) 7.32-7.34 (m, 3H) 7.53 (d, 2H).

### EXAMPLE 3

### 2-Ethyl-5,7-dimethyl-3-[4-(3-pyrrolidin-1-yl-prop-1-ynyl)-benzyl]-3H-imidazo[4,5-b]pyridine

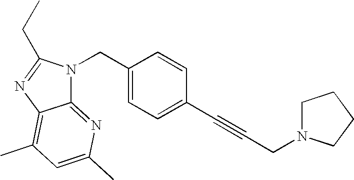

This compound was synthesized analogously to example 1 from 3-[4-(2-ethyl-5,7-dimethyl-imidazo[4,5-b]pyridin-3-ylmethyl)-phenyl]-prop-yn-1-ol (3) and pyrrolidine.

MS (ESI): 373.3 [M+H]⁺, ¹H-NMR (MeOD, 400 MHz) δ (ppm): 1.29 (t, J=7.58 Hz, 3H) 1.89 (t, 4H) 2.63 (d, 6H) 2.76 (t, 4H) 2.88 (d, 2H) 3.65 (s, 2H) 5.59 (s, 2H) 7.06 (s, 1H) 7.12 (d, 2H) 7.41 (d, 2H).

### EXAMPLE 4

### {3-[4-(2-Ethyl-5,7-dimethyl-imidazo[4,5-b]pyridin-3-ylmethyl)-phenyl]-prop-2-ynyl}-(2-pyrrolidin-1-yl-ethyl)-amine

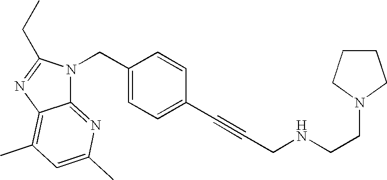

This compound was synthesized analogously to example 1 from 3-[4-(2-ethyl-5,7-dimethyl-imidazo[4,5-b]pyridin-3-ylmethyl)-phenyl]-prop-yn-1-ol (3) and 1-(2-aminoethylpyrrolidine).

MS (ESI): 416 [M+H]⁺, ¹H-NMR (MeOD, 400 MHz) δ (ppm): 1.35 (t, 3H) 2.06-2.17 (m, 4H) 2.66 (d, 6H) 3.16 (q, 2H)) 3.30 (br. s., 2H) 3.45 (br. s., 4H) 3.57 (m, 4H) 4.20 (s, 2H) 5.75 (s, 2H) 7.33-7.35 (m, 3H) 7.50 (d, 2H).

Synthesis of the phenyl-ethyl-amines

The phenylethylamines were Generally Prepared by a Synthesis as Lined Out in Reaction Scheme 11.

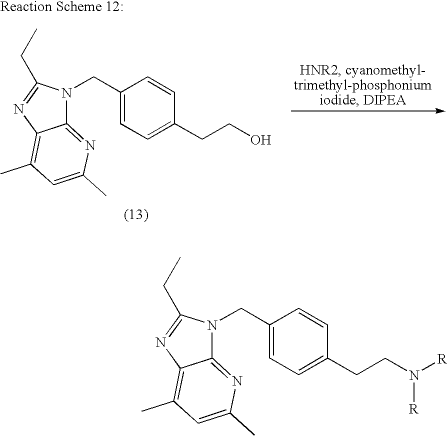

### EXAMPLE 5

### 1′-{2-[4-(2-Ethyl-5,7-dimethyl-imidazo[4,5-b]pyridin-3-ylmethyl)-phenyl]-ethyl}-[1,4′]bipiperidinyl

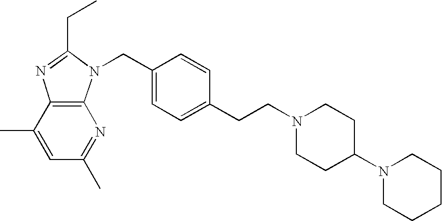

2-[4-(2-Ethyl-5,7-dimethyl-imidazo[4,5-b]pyridin-3-ylmethyl)-phenyl]-ethanol (13) (50 mg, 0.162 mmol) was dissolved in 2 ml of propionitrile and after addition of 4-piperidinopiperidine (27.2 mg, 0.162 mmol), diisopropylethylamine (0.141 ml, 0.808 mmol) and cyanomethyl trimethyl phosphonium iodide (93 mg, 0.404 mmol) the mixture was stirred for 2 h at 100° C. The solvent was evaporated under reduced pressure. The residue was diluted with ethyl acetate, washed with 5% NaHCO₃— and NaCl-solution and dried over Na₂SO₄. Evaporation gave a brown oil which was purified by flash-chromatography (silica gel, ethyl acetate/methanol). The hydrochloride was prepared by adding excess methanolic HCl to the pure product followed by evaporation.

MS (ESI): 460 [M+H]⁺, ¹H-NMR (DMSO-d6, 500 MHz) δ (ppm): 7.25-7.18 (m, 5H), 5.56 (s, 2H), 3.80-2.86 (m, 15H), 2.61 (s, 3H), 2.57 (s, 3H), 2.34-1.32 (m, 10H), 1.24 (t, 3H).

Synthesis of the phenyl-propyl-amines

The phenylpropylamines were Generally Prepared by a Synthesis as Lined Out in Reaction Scheme 12.

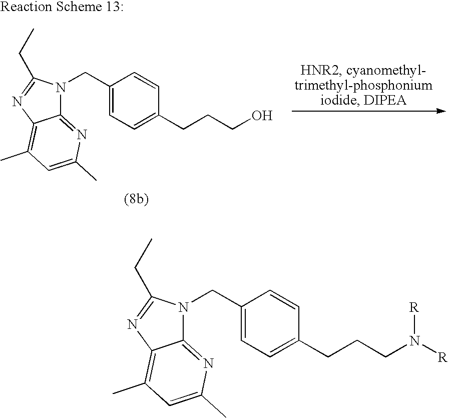

### EXAMPLE 6

### 2-Ethyl-5,7-dimethyl-3-{4-[3-(4-methyl-piperazin-1-yl)-propyl]-benzyl}-3H-imidazo[4,5-b]pyridine dihydrochloride

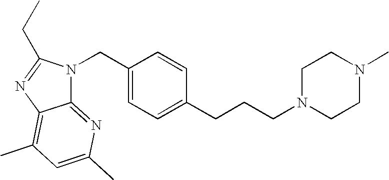

3-[4-(2-Ethyl-5,7-dimethyl-imidazo[4,5-b]pyridin-3-ylmethyl)-phenyl]-propan-1-ol (8b) (100 mg, 0.309 mmol) was dissolved in 0.7 ml of propionitrile and after addition of N-methylpiperazine (0.034 ml, 0.309 mmol), cyanomethyl trimethyl phosphonium iodide (177 mg, 0.773 mmol) and diisopropylethylamine (0.270 ml, 1.546 mmol) the mixture was stirred for 16 h at 100° C. Then the mixture was evaporated under reduced pressure. The residue was diluted with ethyl acetate, washed with 5% NaHCO₃— and NaCl-solution and dried over Na₂SO₄. Evaporation gave a brown oil which was purified by chomatography (silica gel, ethyl acetate/methanol). The hydrochloride was prepared by adding excess methanolic HCl to the pure product followed by evaporation.

MS (ESI): 406 [M+H]⁺, ¹H-NMR (DMSO-d6, 500 MHz) δ (ppm): 11.5-12 (br, 2H), 7.27 (s, 1H), 7.21 (m, 4H), 5.59 (s, 2H), 4.00-3.20 (m, 8H), 3.15-2.95 (m, 4H), 2.79 (s, 3H), 2.65-2.59 (m, 2H), 2.60 (s, 3H), 2.57 (s, 3H), 2.03-1.94 (m, 2H), 1.25 (t, 3H).

### EXAMPLE 7

### 2-Ethyl-5,7-dimethyl-3-{4-[3-(4-propyl-piperazin-1-yl)-propyl]-benzyl}-3H-imidazo[4,5-b]pyridine

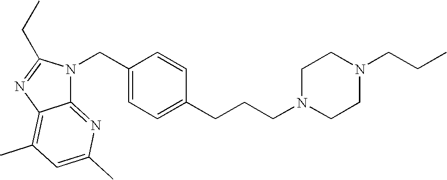

This compound was synthesized analogously to example 6 from N-propyl-piperazine without the formation of the hydrochloride salt.

MS (ESI): 434 [M+H]⁺, ¹H-NMR (DMSO-d6, 500 MHz) δ (ppm): 7.11 (d, 2H), 7.01 (d, 2H), 6.93 (s, 1H), 5.39 (s, 2H), 2.74 (q, 2H), 2.55-2.50 (m, 2H), 2.49 (s, 6H), 2.45-2.25 (m, 8H), 2.22-2.14 (m, 4H), 1.70-1.60 (m, 2H), 1.45-1.35 (m, 2H), 1.19 (t, 3H), 0.81 (t, 3H).

### EXAMPLE 8

### 2-Ethyl-3-{4-[3-(4-isopropyl-piperazin-1-yl)-propyl]-benzyl}-5,7-dimethyl-3H-imidazo[4,5-b]pyridine dihydrochloride

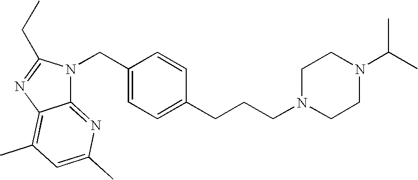

This compound was synthesized analogously to example 6 from 1-isopropyl-piperazine

MS (ESI): 434 [M+H]⁺, ¹H-NMR (DMSO-d6, 500 MHz) δ (ppm): 8.4 (br, 2H), 7.1 (d, 2H), 7.0 (d, 2H), 6.95 (s, 1H), 5.4 (s, 2H), 2.75 (q, 2H), 2.7-2.2 (m, 16H), 1.67 (m, 2H), 1.55 (m, 1H), 1.2 (t, 3H), 1.0 (m, 1H), 0.95 (d, 6H), 0.85 (m, 1H).

### EXAMPLE 9

### 3-{4-[3-(4-Cyclopropyl-piperazin-1-yl)-propyl]-benzyl}-2-ethyl-5,7-dimethyl-3H-imidazo[4,5-b]pyridine

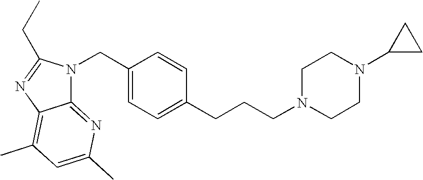

This compound was synthesized analogously to example 6 from 1-cyclopropyl-piperazine without the formation of the hydrochloride salt.

MS (ESI): 432 [M+H]⁺, ¹H-NMR (DMSO-d6, 500 MHz) δ (ppm): 7.30 (s, 1H), 7.23-721 (m, 4H), 5.61 (s, 2H), 3.99-2.60 (m, 15H), 2.62 (s, 3H), 2.58 (s, 3H), 2.02-1.94 (m, 2H), 1.26 (t, 3H), 1.01-0.67 (m, 4H).

### EXAMPLE 10

### 3-{4-[3-(4-Butyl-piperazin-1-yl)-propyl]-benzyl}-2-ethyl-5,7-dimethyl-3H-imidazo[4,5-b]pyridine

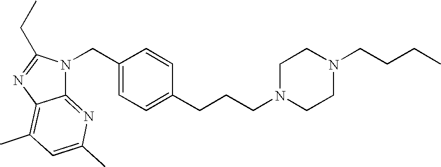

This compound was synthesized analogously to example 6 from 1-butylpiperazine without the formation of the hydrochloride salt.

MS (ESI): 448 [M+H]⁺, ¹H-NMR (DMSO-d6, 500 MHz) δ (ppm): 7.12 (d, 2H), 7.07 (d, 2H), 6.89 (s, 1H), 5.42 (s, 2H), 3.2-2.5 (m, 14H), 2.74 (q, 2H), 2.49 (s, 6H), 1.75-1.65 (m, 2H), 1.5-1.4 (m, 2H), 1.2-1.1.15 (m, 2H), 1.2 (t, 3H), 0.83 (t, 3H).

### EXAMPLE 11

### 3-{4-[3-(4-sec-Butyl-piperazin-1-yl)-propyl]-benzyl}-2-ethyl-5,7-dimethyl-3H-imidazo[4,5-b]pyridine

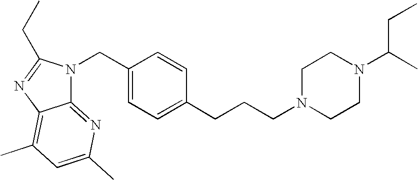

This compound was synthesized analogously to example 6 from 1-sec-butyl-piperazine without the formation of the hydrochloride salt.

MS (ESI): 448 [M+H]⁺, ¹H-NMR (DMSO-d6, 500 MHz) δ (ppm): 7.12 (d, 2H), 7.02 (d, 2H), 6.92 (s, 1H), 5.39 (s, 2H), 3.80-2.90 (m, 6H), 2.74 (q, 2H), 2.49 (s, 6H), 2.48-2.10 (m, 6H), 1.70-1.60 (m, 2H), 1.50-1.40 (m, 2H), 1.35-1.10 (m, 4H), 0.89 (t, 3H), 0.83 (t, 3H).

### EXAMPLE 12

### 3-{4-[3-(4-tert-Butyl-piperazin-1-yl)-propyl]-benzyl}-2-ethyl-5,7-dimethyl-3H-imidazo[4,5-b]pyridine

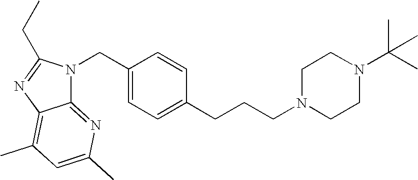

This compound was synthesized analogously to example 6 from 1-tert.butylpiperazine without the formation of the hydrochloride salt.

MS (ESI): 448 [M+H]⁺, ¹H-NMR (DMSO-d6, 500 MHz) δ (ppm): 7.12 (d, 2H), 7.01 (d, 2H), 6.93 (s, 1H), 5.39 (s, 2H), 2.74 (q, 2H), 2.49 (s, 6H), 2.50-2.20 (m, 10H), 2.17 (t, 2H), 1.63 (m, 2H), 1.19 (t, 3H), 0.96 (s, 9H).

### EXAMPLE 13

### 2-Ethyl-5,7-dimethyl-3-{4-[3-(4-pentyl-piperazin-1-yl)-propyl]-benzyl}-3H-imidazo[4,5-b]pyridine

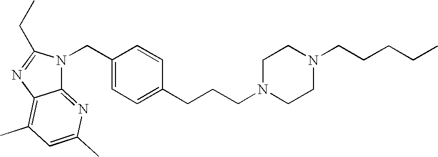

This compound was synthesized analogously to example 6 from 1-pentyl-piperazine without the formation of the hydrochloride salt.

MS (ESI): 462 [M+H]⁺, ¹H-NMR (DMSO-d6, 500 MHz) δ (ppm): 7.13 (d, 2H), 7.02 (d, 2H), 6.93 (s, 1H), 5.39 (s, 2H), 3.45-3.20 (m, 4H), 2.74 (q, 2H), 2.49 (s, 6H), 2.45-2.10 (m, 10H), 1.70-1.60 (m, 2H), 1.35-1.10 (m, 6H), 0.85 (t, 6H).

### EXAMPLE 14

### 2-Ethyl-5,7-dimethyl-3-(4-{3-[4-(1-methyl-butyl)-piperazin-1-yl]-propyl}-benzyl)-3H-imidazo[4,5-b]pyridine

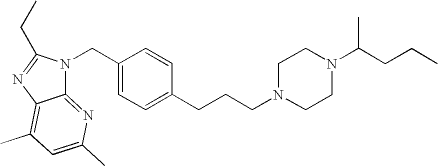

This compound was synthesized analogously to example 6 from 1-(1-methyl-butyl)-piperazine without the formation of the hydrochloride salt.

MS (ESI): 462 [M+H]⁺, ¹H-NMR (DMSO-d6, 500 MHz) δ (ppm): 7.11 (d, 2H), 7.01 (d, 2H), 6.93 (s, 1H), 5.39 (s, 2H), 2.74 (q, 2H), 2.49 (s, 6H), 2.45-2.15 (m, 11H), 1.70-1.60 (m, 2H), 1.55-1.38 (m, 2H), 1.35-1.10 (m, 7H), 0.85 (t, 6H).

### EXAMPLE 15

### 2-Ethyl-3-(4-{3-[4-(1-ethyl-propyl)-piperazin-1-yl]-propyl}-benzyl)-5,7-dimethyl-3H-imidazo[4,5-b]pyridine

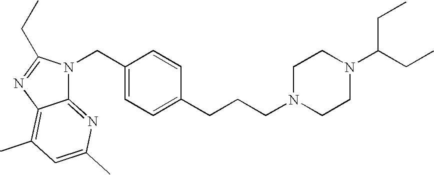

This compound was synthesized analogously to example 6 from 1-(1-ethyl-propyl)-piperazine without the formation of the hydrochloride salt.

MS (ESI): 462 [M+H]⁺, ¹H-NMR (DMSO-d6, 500 MHz) δ (ppm): 7.12 (d, 2H), 7.01 (d, 2H), 6.93 (s, 1H), 5.39 (s, 2H), 2.74 (q, 2H), 2.49 (s, 6H), 2.49-2.05 (m, 13H), 1.70-1.60 (m, 2H), 1.50-1.10 (m, 7H), 0.82 (t, 6H).

### EXAMPLE 16

### 2-Ethyl-5,7-dimethyl-3-(4-{3-[4-(3-methyl-butyl)-piperazin-1-yl]-propyl}-benzyl)-3H-imidazo[4,5-b]pyridine

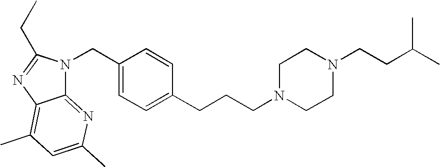

This compound was synthesized analogously to example 6 from 1-(3-methyl-butyl)-piperazine without the formation of the hydrochloride salt.

MS (ESI): 462 [M+H]⁺, ¹H-NMR (DMSO-d6, 500 MHz) δ (ppm): 7.12 (d, 2H), 7.01 (d, 2H), 6.93 (s, 1H), 5.39 (s, 2H), 2.74 (q, 2H), 2.49 (s, 6H), 2.49-2.05 (m, 14H), 1.70-1.60 (m, 2H), 1.58-1.46 (m, 1H), 1.35-1.25 (m, 2H), 1.18 (t, 3H), 0.85 (d, 3H), 0.84 (d, 3H).

### EXAMPLE 17

### 3-{4-[3-(4-Cyclobutylmethyl-piperazin-1-yl)-propyl]-benzyl}-2-ethyl-5,7-dimethyl-3H-imidazo[4,5-b]pyridine dihydrochloride

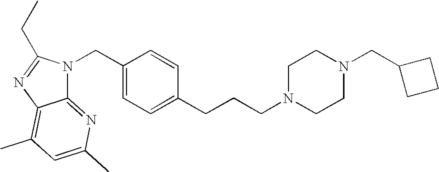

This compound was synthesized analogously to example 6 from 1-(cycobutyl)methyl-piperazine.

MS (ESI): 460 [M+H]⁺, ¹H-NMR (DMSO-d6, 500 MHz) δ (ppm): 11.5-12 (br, 2H), 7.29 (s, 1H), 7.23-721 (m, 4H), 5.61 (s, 2H), 4.20-3.30 (m, 10H), 3.20-3.00 (m, 6H), 2.85-2.65 (m, 1H), 2.62 (s, 3H), 2.58 (s, 3H), 2.20-1.60 (m, 8H), 1.26 (t, 3H).

### EXAMPLE 18

### 3-{4-[3-(4-Cyclopentyl-piperazin-1-yl)-propyl]-benzyl}-2-ethyl-5,7-dimethyl-3H-imidazo[4,5-b]pyridine

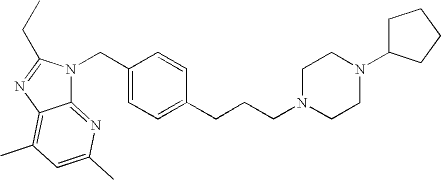

This compound was synthesized analogously to example 6 from 1-cyclopentylpiperazine without the formation of the hydrochloride salt.

MS (ESI): 460 [M+H]⁺, ¹H-NMR (DMSO-d6, 500 MHz) δ (ppm): 7.11 (d, 2H), 7.01 (d, 2H), 6.93 (s, 1H), 5.39 (s, 2H), 2.74 (q, 2H), 2.49 (s, 6H), 2.49-2.10 (m, 12H), 1.80-1.20 (m, 11H), 1.19 (t, 3H).

### EXAMPLE 19

### 3-{4-[3-(4-Cyclohexyl-piperazin-1-yl)-propyl]-benzyl}-2-ethyl-5,7-dimethyl-3H-imidazo[4,5-b]pyridine dihydrochloride

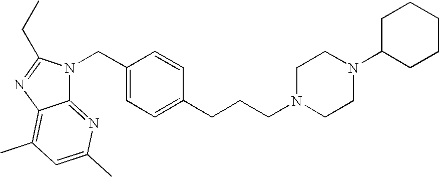

This compound was synthesized analogously to example 6 from 1 -cyclohexylpiperazine.

MS (ESI): 474 [M+H]⁺, ¹H-NMR (DMSO-d6, 500 MHz) δ (ppm): 11.5-12.0 (br, 2H), 7.29 (s, 1H), 7.23-721 (m, 4H), 5.59 (s, 2H), 4.10-3.00 (m, 9H), 2.65-2.60 (m, 2H), 2.60 (s, 3H), 2.57 (s, 3H), 2.15-1.90 (m, 6H), 1.90-1.70 (m, 2H), 1.65-1.55 (m, 2H), 1.50-1.30 (m, 4H), 1.25 (t, 3H), 1.20-1.00 (m, 2H).

### EXAMPLE 20

### 2-Ethyl-3-(4-{3-[4-(2-methoxy-ethyl)-piperazin-1-yl]-propyl}-benzyl)-5,7-dimethyl-3H-imidazo[4,5-b]pyridine dihydrochloride

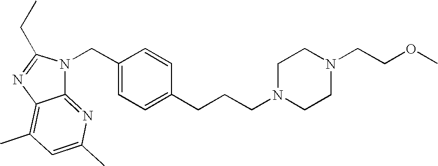

This compound was synthesized analogously to example 6 from 1-(2-methoxyethyl)-piperazine.

MS (ESI): 450 [M+H]⁺, ¹H-NMR (DMSO-d6, 500 MHz) δ (ppm): 11.5-12.0 (br, 2H), 7.32 (s, 1H), 7.23-721 (m, 4H), 5.63 (s, 2H), 3.90-2.85 (m, 18H), 3.28 (s, 3H), 2.64 (s, 3H), 2.58 (s, 3H), 2.10-1.90 (m, 2H), 1.27 (t, 3H).

### EXAMPLE 21

### [2-(4-{3-[4-(2-Ethyl-5,7-dimethyl-imidazo[4,5-b]pyridin-3-ylmethyl)-phenyl]-propyl}-piperazin-1-yl)-ethyl]-dimethyl-amine dihydrochloride

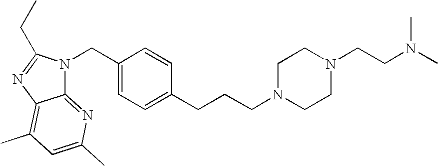

This compound was synthesized analogously to example 6 from 1-(2-dimetylamino-ethyl)piperazine.

MS (ESI): 463 [M+H]⁺, ¹H-NMR (DMSO-d6, 500 MHz) δ (ppm): 12.0 (br, 1H), 10.8 (br, 1H), 9.7 (br, 1H), 7.32 (s, 1H), 7.23-721 (m, 4H), 5.66 (s, 2H), 3.90-2.85 (m, 16H), 2.77 (s, 6H), 2.70-2.55 (m, 2H), 2.50 (s, 6H), 2.10-1.90 (m, 2H), 1.27 (t, 3H).

### EXAMPLE 22

### 2-Ethyl-3-{4-[3-(4-ethyl-3-methyl-piperazin-1-yl)-propyl]-benzyl}-5,7-dimethyl-3H-imidazo[4,5-b]pyridine dihydrochloride

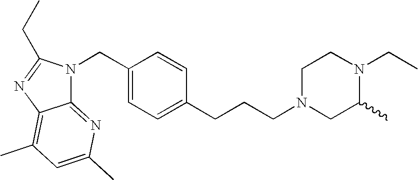

This compound was synthesized analogously to example 6 from 1-ethyl-2-methyl-piperazine.

MS (ESI): 434 [M+H]⁺, ¹H-NMR (DMSO-d6, 500 MHz) δ (ppm): 11.8-12.0 (br, 2H), 10.0 (br, 1H), 7.32 (s, 1H), 7.23-721 (m, 4H), 5.63 (s, 2H), 4.00-2.85 (m, 15H), 2.64 (s, 3H), 2.58 (s, 3H), 2.10-1.90 (m, 2H), 1.30 (d, 3H), 1.27 (t, 3H), 1.21 (t, 3H).

### EXAMPLE 23

### 2-Ethyl-3-{4-[3-(4-isopropyl-3-methyl-piperazin-1-yl)-propyl]-benzyl}-5,7-dimethyl-3H-imidazo[4,5-b]pyridine

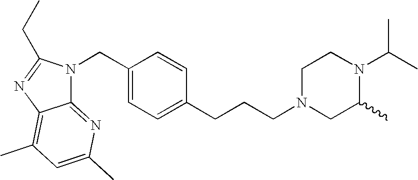

This compound was synthesized analogously to example 6 from 1-isopropyl-2-methylpiperazine without the formation of the hydrochloride salt.

MS (ESI): 448 [M+H]⁺, ¹H-NMR (DMSO-d6, 500 MHz) δ (ppm) (isomeric mixture): 7.12 (d, 2H), 7.01 (d, 2H), 6.93 (s, 1H), 5.39 (s, 2H), 3.20-3.00 (m, 1H), 2.74 (q, 2H), 2.49 (s, 6H), 2.70-1.50 (m, 13H), 1.19 (t, 3H), 1.02 and 0.98 (d, 3H), 0.96 and 0.9 (d, 3H), 0.81 and 0.79 (d, 3H).

### EXAMPLE 24

### 2-Ethyl-3-{4-[3-((S)-2-isopropyl-4-methyl-piperazin-1-yl)-propyl]-benzyl}-5,7-dimethyl-3H-imidazo[4,5-b]pyridine dihydrochloride

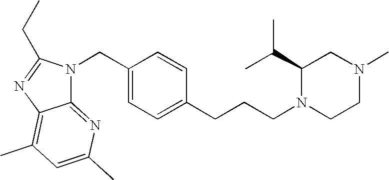

This compound was synthesized analogously to example 6 from (S)-3-isopropyl-1-methylpiperazine.

MS (ESI): 448 [M+H]⁺, ¹H-NMR (DMSO-d6, 500 MHz) δ (ppm): 11.5-12.0 (br, 2H), 7.15 (d, 2H), 7.10 (d, 2H), 6.93 (s, 1H), 5.42 (s, 2H), 3.40-2.60 (m, 15H), 2.81 (q, 2H), 2.54 (s, 3H), 2.53 (s, 3H), 1.80 (m, 2H), 1.27 (t, 3H), 0.93 (d, 3H), 0.85 (d, 3H).

### EXAMPLE 25

### 2-Ethyl-3-{4-[3-(3-ethyl-4-isopropyl-piperazin-1-yl)-propyl]-benzyl}-5,7-dimethyl-3H-imidazo[4,5-b]pyridine

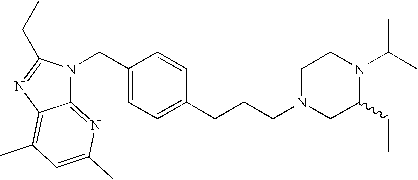

This compound was synthesized analogously to example 6 from 2-ethyl-1-isopropylpiperazine without the formation of the hydrochloride salt.

MS (ESI): 462 [M+H]⁺, ¹H-NMR (DMSO-d6, 500 MHz) δ (ppm): 7.29 (s, 1H), 7.25-721 (m, 4H), 5.61 (s, 2H), 4.00-3.25 (m, 10H), 3.20-3.00 (m, 4H), 2.64 (s, 3H), 2.58 (s, 3H), 2.10-1.90 (m, 3H), 1.80-1.60 (m, 1H), 1.37 (d, 3H), 1.27 (t, 3H), 1.13 (d, 3H), 0.94 (t, 3H).

### EXAMPLE 26

### 2-Ethyl-3-{4-[3-((1R,4R)-5-ethyl-2,5-diaza-bicyclo[2.2.1]hept-2-yl)-propyl]-benzyl}-5,7-dimethyl-3H-imidazo[4,5-b]pyridine

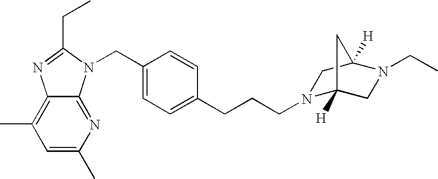

This compound was synthesized analogously to example 6 from (1R,4R)-2-ethyl-2,5-diazabicyclo[2.2.1]heptane without the formation of the hydrochloride salt.

MS (ESI): 432 [M+H]⁺, ¹H-NMR (DMSO-d6, 500 MHz) δ (ppm): 7.1 (d, 2H), 7.0 (d, 2H), 6.9 (s, 1H), 5.4 (s, 2H), 3.45-3.30 (m, 2H), 2.74 (q, 2H), 2.49 (s, 6H), 2.70-2.25 (m, 10H), 1.70-1.50 (m, 4H), 1.19 (t, 3H), 0.94 (t, 3H).

### EXAMPLE 27

### 2-Ethyl-3-{4-[3-((1R,4R)-5-isobutyl-2,5-diaza-bicyclo[2.2.1]hept-2-yl)-propyl]-benzyl}-5,7-dimethyl-3H-imidazo[4,5-b]pyridine dihydrochloride

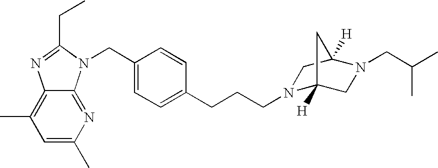

This compound was synthesized analogously to example 6 from (1R,4R)-2-isobutyl-2,5-diazabicyclo[2.2.1]-heptane.

MS (ESI): 460 [M+H]⁺, ¹H-NMR (DMSO-d6, 500 MHz) δ (ppm): 12.0 (br, 2H), 7.29 (s, 1H), 7.25-7.21 (m, 4H), 5.6 (s, 2H), 4.5 (m, 2H), 4.00-3.25 (m, 9H), 3.20-3.00 (m, 4H), 2.64 (s, 3H), 2.58 (s, 3H), 2.10-1.90 (m, 3H), 1.80-1.60 (m, 1H), 1.30 (t, 3H), 1.0 (br s, 3H), 0.90 (d, 3H).

### EXAMPLE 28

### 3-{4-[3-((1R,4R)-5-Cyclopropylmethyl-2,5-diaza-bicyclo[2.2.1]hept-2-yl)-propyl]-benzyl}-2-ethyl-5,7-dimethyl-3H-imidazo[4,5-b]pyridine

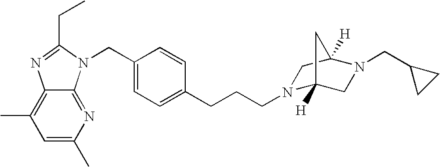

This compound was synthesized analogously to example 6 from (1R,4R)-2-(cyclopropylmethyl)-2,5-diazabicyclo[2.2.1]heptane without the formation of the hydrochloride salt.

MS (ESI): 432 [M+H]⁺, ¹H-NMR (DMSO-d6, 500 MHz) δ (ppm): 7.11 (d, 2H), 7.01 (d, 2H), 6.93 (s, 1H), 5.39 (s, 2H), 3.45-3.30 (m, 2H), 2.74 (q, 2H), 2.49 (s, 6H), 2.70-2.25 (m, 10H), 1.70-1.50 (m, 4H), 1.19 (t, 3H), 0.80-0.70 (m, 1H), 0.45-0.00 (m, 4H).

Synthesis of the phenyl-allyl-amines

The phenyl-allyl-amines were Generally Prepared by a Synthesis as Lined Out in Reaction Scheme 14.

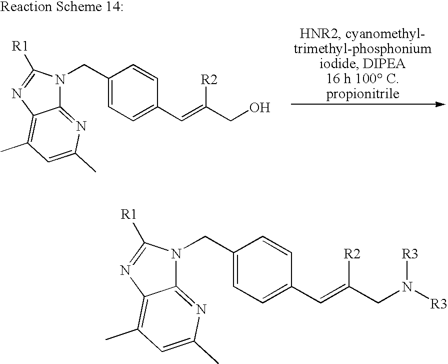

### EXAMPLE 29

### 3-{4-[(E)-3-(4-Isopropyl-piperazin-1-yl)-propenyl]-benzyl}-2,5,7-trimethyl-3H-imidazo[4,5-b]pyridine

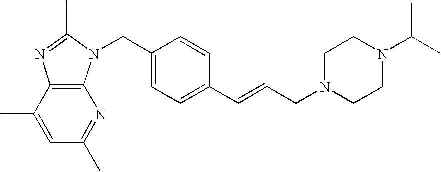

This compound was synthesized analogously to example 6 from alcohol 7a and 1-isopropyl-piperazine without the formation of the hydrochloride salt.

MS (ESI): 418 [M+H]⁺, ¹H-NMR (DMSO-d6, 500 MHz) δ (ppm): 7.35 (d, 2H), 7.05 (d, 2H), 6.92 (s, 1H), 6.45 (d, 1H), 6.22 (dt, 1H), 5.40 (s, 2H), 3.02 (d, 2H), 2.56 (m, 1H), 2.45 (s, 6H), 2.4 (s, 3H), 2.5-2.2 (m, 8H), 0.93 (d, 6H).

### EXAMPLE 30

### 1′-{(E)-3-[4-(2,5,7-Trimethyl-imidazo[4,5-b]pyridin-3-ylmethyl)-phenyl]-allyl}-[1,4′]bipiperidinyl

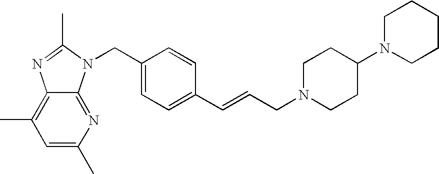

This compound was synthesized analogously to example 6 from alcohol 7a and 4-piperidinopiperidine without the formation of the hydrochloride salt.

MS (ESI): 458 [M+H]⁺, ¹H-NMR (DMSO-d6, 500 MHz) δ (ppm): 7.35 (d, 2H), 7.07 (d, 2H), 6.92 (s, 1H), 6.44 (d, 1H), 6.22 (dt, 1H), 5.40 (s, 2H), 3.01 (d, 2H), 2.84 (d, 2H), 2.45 (s, 6H), 2.4 (s, 3H), 2.39 (m, 4H), 2.15-2.05 (m, 1H), 1.84 (t, 2H), 1.61 (m, 2H), 1.43 (m, 4H), 1.40 (dd, 2H), 1.33 (m, 2H).

### EXAMPLE 31

### Diethyl-(1-{(E)-3-[4-(2-ethyl-5,7-dimethyl-imidazo[4,5-b]pyridin-3-ylmethyl)-phenyl]-allyl}-piperidin-4-yl)-amine dihydrochloride

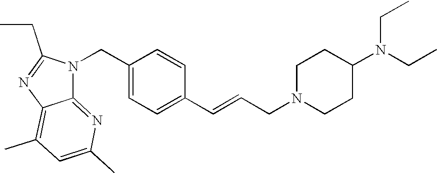

This compound was synthesized analogously to example 6 from alcohol 7b and diethyl-piperidin-4-yl-amine.

MS (ESI): 460 [M+H]⁺, ¹H-NMR (DMSO-d6, 500 MHz) δ (ppm): 11.5 (br, 1H), 10.7 (br, 1H), 7.49 (d, 2H), 7.31 (s, 1H), 7.30 (d, 2H), 6.82 (d, 1H), 6.41 (dt, 1H), 5.67 (s, 2H), 3.84 (m, 2H), 3.4-3.6 (m, 5H), 3.0-3.3 (m, 6H), 2.65 (s, 3H), 2.59 (s, 3H), 2.1-2,45 (m, 4H), 1.28 (t, 9H).

### EXAMPLE 32

### 2-Ethyl-5,7-dimethyl-3-{4-[(E)-3-(4-pyrrolidin-1-yl-piperidin-1-yl)-propenyl]-benzyl}-3H-imidazo[4,5-b]pyridine

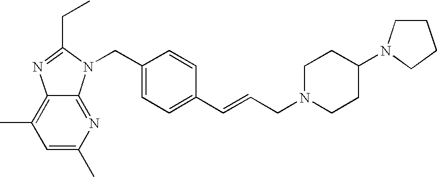

This compound was synthesized analogously to example 6 from alcohol 7b and 4-pyrrolidin-1-yl-piperidine without the formation of the hydrochloride salt.

MS (ESI): 458 [M+H]⁺, ¹H-NMR (DMSO-d6, 500 MHz) δ (ppm): 7.37 (d, 2H), 7.07 (d, 2H), 6.96 (s, 1H), 6.45 (d, 1H), 6.27 (dt, 1H), 5.43 (s, 2H), 3.02 (d, 2H), 2.7-2.85 (m, 4H), 2.52 (s, 3H), 2.51 (s, 3H), 2.44 (m, 4H), 1.85-1.95 (m, 3H), 1.75-1.8 (m, 2H), 1.65 (m, 4H), 1.3-1.4 (m, 2H), 1.22 (t, 3H).

### EXAMPLE 33

### 1′-{(E)-3-[4-(2-Ethyl-5,7-dimethyl-imidazo[4,5-b]pyridin-3-ylmethyl)-phenyl]-allyl}-[1,4′]bipiperidinyl dihydrochloride

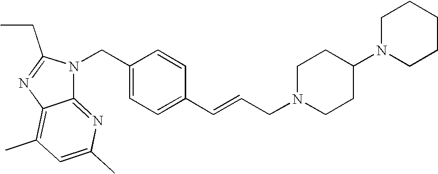

This compound was synthesized analogously to example 6 from alcohol 7b and 4-piperidino-piperidine.

MS (ESI): 472 [M+H]⁺, ¹H-NMR (DMSO-d6, 500 MHz) δ (ppm): 11.1 (br, 1H), 10.7 (br, 1H), 7.45 (d, 2H), 7.28 (s, 1H), 7.27 (d, 2H), 6.75 (d, 1H), 6.39 (dt, 1H), 5.64 (s, 2H), 3.82 (m, 2H), 2.8-3.6 (m, 11H), 2.63 (s, 3H), 2.58 (s, 3H), 2.31 (m, 2H), 2.15 (m, 2H), 1.3-1.9 (m, 6H), 1.27 (t, 3H).

### EXAMPLE 34

### 2-Ethyl-5,7-dimethyl-3-{4-[(E)-3-(4-morpholin-4-yl-piperidin-1-yl)-propenyl]-benzyl}-3H-imidazo[4,5-b]pyridine dihydrochloride

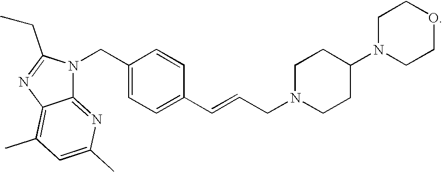

This compound was synthesized analogously to example 6 from alcohol 7b and 4-piperidin-4-yl-morpholine.

MS (ESI): 474 [M+H]⁺, ¹H-NMR (DMSO-d6, 500 MHz) δ (ppm): 11.6 (br, 1H), 10.9 (br, 1H), 7.47 (d, 2H), 7.19 (d, 2H), 7.05 (s, 1H), 6.78 (d, 1H), 6.39 (dt, 1H), 5.52 (s, 2H), 3.8-4.1 (m, 6H), 3.59 (m, 2H), 3.2-3.5 (m, 3H), 2.8-3.2 (m, 6H), 2.56 (s, 3H), 2.53 (s, 3H), 2.1-2.45 (m, 4H), 1.25 (t, 3H).

### EXAMPLE 35

### 2-Ethyl-5,7-dimethyl-3-(4-{(E)-3-[4-(4-methyl-piperazin-1-yl)-piperidin-1-yl]-propenyl}-benzyl)-3H-imidazo[4,5-b]pyridine

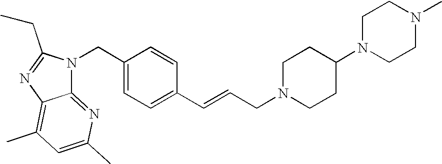

This compound was synthesized analogously to example 6 from alcohol 7b and 1-methyl-4-(piperidin-4-yl)-piperazine without the formation of the hydrochloride salt.

MS (ESI): 487 [M+H]⁺, ¹H-NMR (DMSO-d6, 500 MHz) δ (ppm): 7.47 (d, 2H), 7.27 (m, 3H), 6.78 (d, 1H), 6.39 (dt, 1H), 5.62 (s, 2H), 3.83 (m, 2H), 2.79 (s, 3H), 2.6 (s, 3H), 2.56 (s, 3H), 2.0-3.6 (m, 19H), 1.25 (t, 3H).

### EXAMPLE 36

### 1-{(E)-3-[4-(2-Ethyl-5,7-dimethyl-imidazo[4,5-b]pyridin-3-ylmethyl)-phenyl]-allyl}-piperidin-4-ol

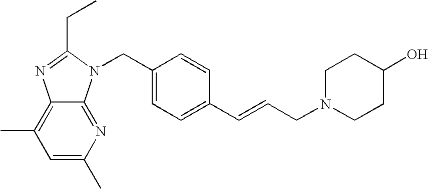

This compound was synthesized analogously to example 6 from alcohol 7b and 4-hydroxypiperidine without the formation of the hydrochloride salt.

MS (ESI): 405 [M+H]⁺, ¹H-NMR (DMSO-d6, 500 MHz) δ (ppm): 7.35 (d, 2H), 7.05 (d, 2H), 6.93 (s, 1H), 6.42 (d, 1H), 6.22 (dt, 1H), 5.41 (s, 2H), 4.51 (s, br, 1H), 3.48-3.35 (m, 1H), 3.00 (d, 2H), 2.76 (q, 2H), 2.70-2.60 (m, 2H), 2.49 (s, 6H), 1.99 (t, 2H), 1.75-1.60 (m, 2H), 1.45-1.30 (m, 2H), 1.20 (t, 3H).

### EXAMPLE 37

### 2-Ethyl-3-{4-[(E)-3-(4-methoxy-piperidin-1-yl)-propenyl]-benzyl}-5,7-dimethyl-3H-imidazo[4,5-b]pyridine

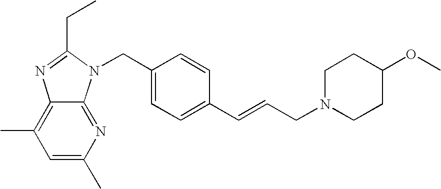

This compound was synthesized analogously to example 6 from alcohol 7b and 4-methoxypiperidine without the formation of the hydrochloride salt.

MS (ESI): 419 [M+H]⁺, ¹H-NMR (DMSO-d6, 500 MHz) δ (ppm): 7.35 (d, 2H), 7.05 (d, 2H), 6.93 (s, 1H), 6.45 (d, 1H), 6.23 (dt, 1H), 5.41 (s, 2H), 3.19 (s, 3H), 3.12 (m, 1H), 3.02 (d, 2H), 2.74 (q, 2H), 2.70-2.60 (m, 2H), 2.49 (s, 6H), 2.10-1.95 (m, 2H), 1.85-1.75 (m, 2H), 1.45-1.30 (m, 2H), 1.20 (t, 3H).

### EXAMPLE 38

### 2-(1-{(E)-3-[4-(2-Ethyl-5,7-dimethyl-imidazo[4,5-b]pyridin-3-ylmethyl)-phenyl]-allyl}-piperidin-4-yloxy)-N-methyl-acetamide

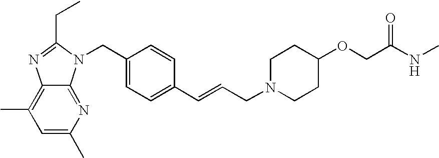

This compound was synthesized analogously to example 6 from alcohol 7b and N-methyl-2-(piperidin-4-yloxy)acetamide without the formation of the hydrochloride salt.

MS (ESI): 476 [M+H]⁺, ¹H-NMR (DMSO-d6, 500 MHz) δ (ppm): 7.54 (s, br, 1H), 7.35 (d, 2H), 7.05 (d, 2H), 6.93 (s, 1H), 6.44 (d, 1H), 6.24 (dt, 1H), 5.41 (s, 2H), 3.81 (s, 2H), 3.45-3.10 (m, 1H), 3.02 (d, 2H), 2.74 (q, 2H), 2.70-2.61 (m, 2H), 2.60 (d, 3H), 2.49 (s, 6H), 2.10-1.95 (m, 2H), 1.80-1.60 (m, 2H), 1.55-1.35 (m, 2H), 1.20 (t, 3H).

### EXAMPLE 39

### 1-{(E)-3-[4-(2-Ethyl-5,7-dimethyl-imidazo[4,5-b]pyridin-3-ylmethyl)-phenyl]-allyl}-4-morpholin-4-ylmethyl-piperidin-4-ol

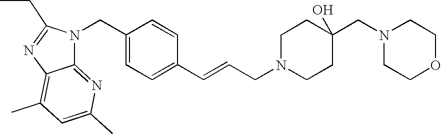

This compound was synthesized analogously to example 6 from alcohol 7b and 4-(morpholinomethyl)piperidin-4-ol without the formation of the hydrochloride salt.

MS (ESI): 504 [M+H]⁺, ¹H-NMR (DMSO-d6, 500 MHz) δ (ppm): 7.35 (d, 2H), 7.05 (d, 2H), 6.93 (s, 1H), 6.44 (d, 1H), 6.24 (dt, 1H), 5.41 (s, 2H), 3.91 (s, 1H), 3.52 (t, 4H), 3.03 (d, 2H), 2.74 (q, 2H), 2.49 (s, 6H), 2.49-2.35 (m, 6H), 2.28 (m, 2H), 2.18 (s, 2H), 1.52 (m, 2H), 1.48-1.30 (m, 2H), 1.20 (t, 3H).

### EXAMPLE 40

### 2-Ethyl-3-(4-{(E)-3-[4-(1H-indol-3-yl)-piperidin-1-yl]-propenyl}-benzyl)-5,7-dimethyl-3H-imidazo[4,5-b]pyridine hydrochloride

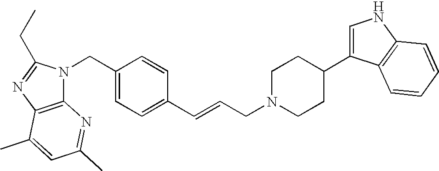

This compound was synthesized analogously to example 6 from alcohol 7b and 3-piperidin-4-yl-1H-indole.

MS (ESI): 504 [M+H]⁺, ¹H-NMR (DMSO-d6, 500 MHz) δ (ppm): 10.74 (s, 1H), 7.52 (d, 1H), 7.39 (d, 2H), 7.31 (d, 1H), 7.05 (m, 4H), 6.95 (s, 1H), 6.94 (d, 1H), 6.52 (d, 1H), 6.32 (dt, 1H), 5.44 (s, 2H), 3.12 (d, 2H), 2.97 (m, 2H), 2.77 (q, 2H), 2.72 (m, 1H), 2.52 (s, 6H), 2.12 (m, 2H), 1.93 (m, 2H), 1.72 (m, 2H), 1.24 (t, 3H).

### EXAMPLE 41

### 3-{4-[(E)-3-((1R,5S)-3-Benzoimidazol-1-yl-8-aza-bicyclo[3.2.1]oct-8-yl)-propenyl]-benzyl}-2-ethyl-5,7-dimethyl-3H-imidazo[4,5-b]pyridine hydrochloride

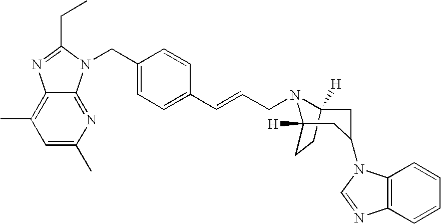

This compound was synthesized analogously to example 6 from alcohol 7b and 1-(8-azabicyclo(3.2.1)oct-3yl)-1H-benzimidazol.

MS (ESI): 531 [M+H]⁺, ¹H-NMR (DMSO-d6, 500 MHz) δ (ppm): 11.95 (br s, 1H), 9.75 (s, 1H), 8.75 (d, 1H), 7.9 (m, 1H), 7.62 (m, 2H), 7.49 (d, 2H), 7.36 (s, 1H), 7.35 (d, 2H), 6.92 (s, 1H), 6.60 (dt, 1H), 5.71 (s, 2H), 5.44 (m, 1H), 4.19 (m, 1H), 3.85 (m, 1H), 3.19 (q, 2H), 3.04 (m, 2H), 2.67 (s, 3H), 2.60 (s, 3H), 2.31 (m, 8H), 1.29 (t, 3H).

### EXAMPLE 42

### 2-Ethyl-5,7-dimethyl-3-[4-((E)-3-piperazin-1-yl-propenyl)-benzyl]-3H-imidazo[4,5-b]pyridine

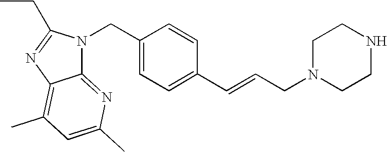

This compound was synthesized analogously to example 6 from alcohol 7b and piperazine without the formation of the hydrochloride salt.

MS (ESI): 390 [M+H]⁺, ¹H-NMR (DMSO-d6, 500 MHz) δ (ppm): 7.35 (d, 2H), 7.05 (d, 2H), 6.93 (s, 1H), 6.45 (d, 1H), 6.23 (dt, 1H), 5.41 (s, 2H), 3.01 (d, 2H), 2.76 (q, 2H), 2.75-2.65 (m, 4H), 2.49 (s, 6H), 2.40-2.20 (m, 4H), 1.20 (t, 3H).

### EXAMPLE 43

### 2-Ethyl-5,7-dimethyl-3-{4-[(E)-3-(4-methyl-piperazin-1-yl)-propenyl]-benzyl}-3H-imidazo[4,5-b]pyridine

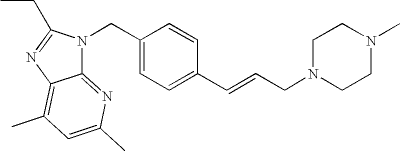

This compound was synthesized analogously to example 6 from alcohol 7b and 1-methylpiperazine without the formation of the hydrochloride salt.

MS (ESI): 404 [M+H]⁺, ¹H-NMR (DMSO-d6, 500 MHz) δ (ppm): 7.36 (d, 2H), 7.05 (d, 2H), 6.93 (s, 1H), 6.47 (d, 1H), 6.23 (dt, 1H), 5.41 (s, 2H), 3.05 (d, 2H), 2.76 (q, 2H), 2.50 (s, 6H), 2.50-2.15 (m, 8H), 2.17 (s, 3H), 1.20 (t, 3H).

### EXAMPLE 44

### 2-Ethyl-3-{4-[(E)-3-(4-ethyl-piperazin-1-yl)-propenyl]-benzyl}-5,7-dimethyl-3H-imidazo[4,5-b]pyridine dihydrochloride

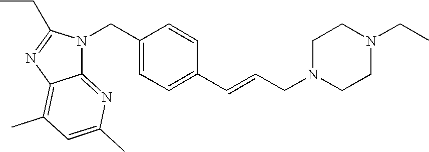

This compound was synthesized analogously to example 6 from alcohol 7b and N-ethylpiperazine

MS (ESI): 418 [M+H]⁺, ¹H-NMR (DMSO-d6, 500 MHz) δ (ppm): 12.0 (br, 2H), 7.45 (d, 2H), 7.29 (d, 2H), 7.28 (s, 1H), 6.85 (d, 1H), 6.38 (dt, 1H), 5.66 (s, 2H), 3.95-3.85 (m, 2H), 3.70-2.96 (m, 12H), 2.63 (s, 3H), 2.57 (s, 3H), 1.26 (t, 3H), 1.08 (t, 3H).

### EXAMPLE 45

### 2-Ethyl-5,7-dimethyl-3-{4-[(E)-3-(4-propyl-piperazin-1-yl)-propenyl]-benzyl}-3H-imidazo[4,5-b]pyridine

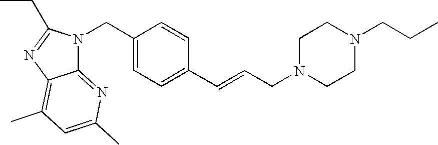

This compound was synthesized analogously to example 6 from alcohol 7b and 1-propylpiperazine without the formation of the hydrochloride salt.

MS (ESI): 432 [M+H]⁺, ¹H-NMR (DMSO-d6, 500 MHz) δ (ppm): 7.36 (d, 2H), 7.05 (d, 2H), 6.93 (s, 1H), 6.45 (d, 1H), 6.22 (dt, 1H), 5.41 (s, 2H), 3.03 (d, 2H), 2.75 (q, 2H), 2.50 (s, 6H), 2.50-2.15 (m, 8H), 2.17 (t, 2H), 1.39 (t, 2H), 1.20 (t, 3H), 0.81 (t, 3H).

### EXAMPLE 46

### 2-Ethyl-3-{4-[(E)-3-(4-isopropyl-piperazin-1-yl)-propenyl]-benzyl}-5,7-dimethyl-3H-imidazo[4,5-b]pyridine

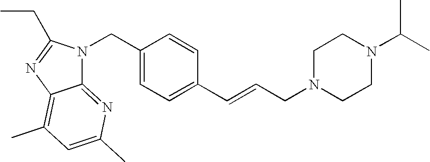

This compound was synthesized analogously to example 6 from alcohol 7b and 1-isopropyl-piperazine without the formation of the hydrochloride salt.

MS (ESI): 432 [M+H]⁺, ¹H-NMR (DMSO-d6): δ (ppm) 7.37 (d, 2H), 7.05 (d, 2H), 6.94 (s, 1H), 6.46 (d, 1H), 6.24 (dt, 1H), 5.43 (s, 2H), 3.03 (d, 2H), 2.77 (q, 2H), 2.59 (h, 1H), 2.52 (s, 3H), 2.51 (s, 3H), 2.42 (m, 8H), 1.23 (t, 3H), 0.95 d, 6H).

### EXAMPLE 47

### 3-{4-[(E)-3-(4-Cyclopropyl-piperazin-1-yl)-propenyl]-benzyl}-2-ethyl-5,7-dimethyl-3H-imidazo[4,5-b]pyridine dihydrochloride

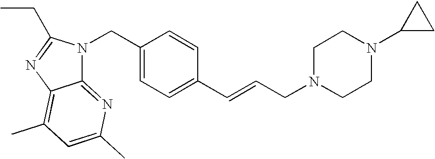

This compound was synthesized analogously to example 6 from alcohol 7b and 1-cyclopropylpiperazine.

MS (ESI): 430 [M+H]⁺, ¹H-NMR (DMSO-d6, 500 MHz) δ (ppm): 12.0 (br, 2H), 7.46 (d, 2H), 7.31 (s, 1H), 7.29 (d, 2H), 6.86 (d, 1H), 6.38 (dt, 1H), 5.67 (s, 2H), 3.95-3.85 (m, 2H), 3.70-3.25 (m, 9H), 3.20-3.05 (m, 2H), 2.64 (s, 3H), 2.58 (s, 3H), 1.27 (t, 3H), 1.20-0.60 (m, 4H).

### EXAMPLE 48

### 3-{4-[(E)-3-(4-Butyl-piperazin-1-yl)-propenyl]-benzyl}-2-ethyl-5,7-dimethyl-3H-imidazo[4,5-b]pyridine dihydrochloride

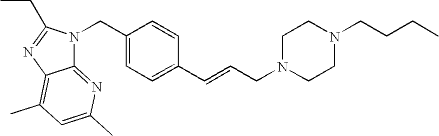

This compound was synthesized analogously to example 6 from alcohol 7b and 1-butylpiperazine.

MS (ESI): 446 [M+H]⁺, ¹H-NMR (DMSO-d6, 500 MHz) δ (ppm): 12.5-11.8 (br, 2H), 7.45 (d, 2H), 7.29 (d, 2H), 7.28 (s, 1H), 6.86 (d, 1H), 6.36 (dt, 1H), 5.66 (s, 2H), 4.00-3.85 (m, 2H), 3.70-3.25 (m, 8H), 3.20-2.90 (m, 4H), 2.64 (s, 3H), 2.57 (s, 3H), 1.75-1.60 (m, 2H), 1.40-1.25 (m, 2H), 1.26 (t, 3H), 0.89 (t, 3H).

### EXAMPLE 49

### 3-{4-[(E)-3-(4-sec-Butyl-piperazin-1-yl)-propenyl]-benzyl}-2-ethyl-5,7-dimethyl-3H-imidazo[4,5-b]pyridine

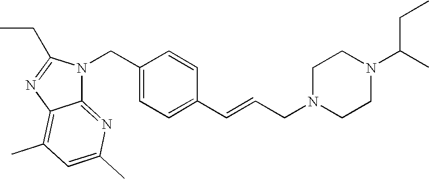

This compound was synthesized analogously to example 6 from alcohol 7b and 1-sec-butylpiperazine without the formation of the hydrochloride salt.

MS (ESI): 446 [M+H]⁺, ¹H-NMR (DMSO-d6, 500 MHz) δ (ppm): 7.36 (d, 2H), 7.05 (d, 2H), 6.93 (s, 1H), 6.47 (d, 1H), 6.23 (dt, 1H), 5.41 (s, 2H), 3.15-3.00 (m, 2H), 2.75 (q, 2H), 2.50 (s, 6H), 2.65-2.25 (m, 8H), 1.55-1.30 (m, 2H), 1.20 (t, 3H), 1.08 (t, 3H). 1.00-0-75 (m, 4H).

### EXAMPLE 50

### 2-Ethyl-3-{4-[(E)-3-(4-isobutyl-piperazin-1-yl)-propenyl]-benzyl}-5,7-dimethyl-3H-imidazo[4,5-b]pyridine dihydrochloride

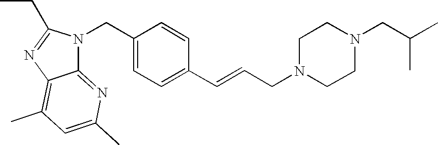

This compound was synthesized analogously to example 6 from alcohol 7b and 1-(2-methylpropyl)piperazine.

MS (ESI): 446 [M+H]⁺, ¹H-NMR (DMSO-d₆): δ (ppm) 12.5 (br, 1H), 11.3 (br, 1H), 7.48 (d, 2H), 7.29 (s, 1H), 7.28 (d, 2H), 6.86 (d, 1H), 6.37 (dt, 1H), 5.65 (s, 2H), 2.8-4.0 (m, 14H), 2.63 (s, 3H), 2.58 (s, 3H), 2.07 (m, 1H), 1.26 (t, 3H), 1.00 (s, 3H), 0.98 (s, 3H).

### EXAMPLE 51

### 3-{4-[(E)-3-(4-tert-Butyl-piperazin-1-yl)-propenyl]-benzyl}-2-ethyl-5,7-dimethyl-3H-imidazo[4,5-b]pyridine

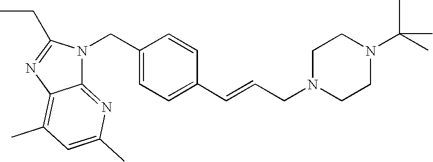

This compound was synthesized analogously to example 6 from alcohol 7b and 1-tert-butylpiperazine without the formation of the hydrochloride salt.

MS (ESI): 446 [M+H]⁺, ¹H-NMR (DMSO-d6, 500 MHz) δ (ppm): 7.36 (d, 2H), 7.04 (d, 2H), 6.93 (s, 1H), 6.44 (d, 1H), 6.23 (dt, 1H), 5.41 (s, 2H), 3.02 (d, 2H), 2.75 (q, 2H), 2.49 (s, 6H), 2.65-2.25 (m, 8H), 1.20 (t, 3H), 0.97 (s, 9H).

### EXAMPLE 52

### 2-Ethyl-5,7-dimethyl-3-(4-{(E)-3-[4-(3-methyl-butyl)-piperazin-1-yl]-propenyl}-benzyl)-3H-imidazo[4,5-b]pyridine dihydrochloride

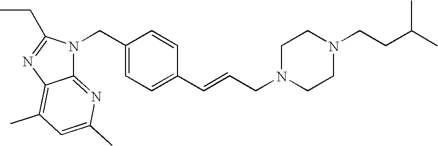

This compound was synthesized analogously to example 6 from alcohol 7b and 1-isopentylpiperazine.

MS (ESI): 460 [M+H]⁺, ¹H-NMR (DMSO-d6, 500 MHz) δ (ppm): 12.5-11.5 (br, 2H), 7.36 (d, 2H), 7.04 (d, 2H), 6.94 (s, 1H), 6.45 (d, 1H), 6.23 (dt, 1H), 5.41 (m, 2H), 3.02 (d, 2H), 2.7-2.8 (m, 2H), 2.50 (s, 6H), 2.5-2.1 (m, 10H), 1.55-1.45 (m, 1H), 1.30-1.10 (m, 5H), 0.84 (d, 6H).

### EXAMPLE 53

### 2-Ethyl-3-(4-{(E)-3-[4-(1-ethyl-propyl)-piperazin-1-yl]-propenyl}-benzyl)-5,7-dimethyl-3H-imidazo[4,5-b]pyridine dihydrochloride

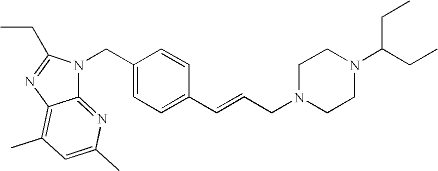

This compound was synthesized analogously to example 6 from alcohol 7b and 1-(3-pentyl)-piperazine.

MS (ESI): 460 [M+H]⁺, ¹H-NMR (DMSO-d6, 500 MHz) δ (ppm): 12.5-11.5 (br, 2H), 7.36 (d, 2H), 7.04 (d, 2H), 6.93 (s, 1H), 6.45 (d, 1H), 6.23 (dt, 1H), 5.41 (s, 2H), 3.02 (d, 2H), 2.75 (q, 2H), 2.50 (s, 3H), 2.49 (s, 3H), 2.50-2.25 (m, 8H), 2.08 (quint., 1H), 1.50-1.10 (m, 4H), 1.20 (t, 3H), 0.82 (t, 6H).

### EXAMPLE 54

### 2-Ethyl-5,7-dimethyl-3-(4-{(E)-3-[4-(1-methyl-butyl)-piperazin-1-yl]-propenyl}-benzyl)-3H-imidazo[4,5-b]pyridine dihydrochloride

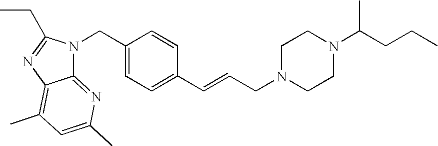

This compound was synthesized analogously to example 6 from alcohol 7b and 1-(2-pentyl)-piperazine.

MS (ESI): 460 [M+H]⁺, ¹H-NMR (DMSO-d6, 500 MHz) δ (ppm): 12.5-11.75 (br, 2H), 7.46 (d, 2H), 7.28 (s, 1H), 7.27 (d, 2H), 6.87 (d, 1H), 6.35 (dt, 1H), 5.65 (s, 2H), 4.00-3.85 (m, 2H), 3.70-3.25 (m, 8H), 3.20-2.90 (m, 2H), 2.62 (s, 3H), 2.57 (s, 3H), 1.60-1.00 (m, 11H), 0.89 (t, 3H).

### EXAMPLE 55

### 3-{4-[(E)-3-(4-Cyclobutylmethyl-piperazin-1-yl)-propenyl]-benzyl}-2-ethyl-5,7-dimethyl-3H-imidazo[4,5-b]pyridine dihydrochloride

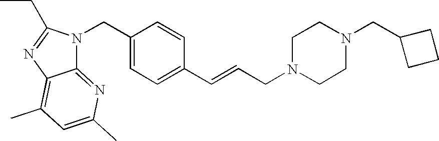

This compound was synthesized analogously to example 6 from alcohol 7b and 1-(cyclobutylmethyl)piperazine.

MS (ESI): 458 [M+H]⁺, ¹H-NMR (DMSO-d6, 500 MHz) δ (ppm): 12.5-11.75 (br, 2H), 7.45 (d, 2H), 7.28 (s, 1H), 7.27 (d, 2H), 6.87 (d, 1H), 6.35 (dt, 1H), 5.65 (s, 2H), 4.00-3.85 (m, 2H), 3.80-3.25 (m, 8H), 3.20-3.00 (m, 4H), 2.77 (m, 1H), 2.63 (s, 3H), 2.57 (s, 3H), 2.15-1.70 (m, 6H), 1.26 (t, 3H).

### EXAMPLE 56

### 3-{4-[(E)-3-(4-Cyclopentyl-piperazin-1-yl)-propenyl]-benzyl}-2-ethyl-5,7-dimethyl-3H-imidazo[4,5-b]pyridine

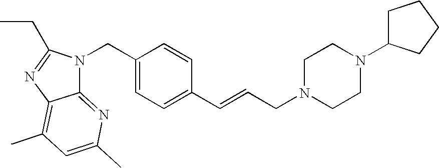

This compound was synthesized analogously to example 6 from alcohol 7b and 1-cyclopentylpiperazine without the formation of the hydrochloride salt.

MS (ESI): 458 [M+H]⁺, ¹H-NMR (DMSO-d6, 500 MHz) δ (ppm): 7.35 (d, 2H), 7.05 (d, 2H), 6.93 (s, 1H), 6.45 (d, 1H), 6.23 (dt, 1H), 5.41 (s, 2H), 3.50-3-40 (m, 1H), 3.02 (d, 2H), 2.75 (q, 2H), 2.50 (s, 6H), 2.65-2.25 (m, 8H), 1.60-1.20 (m, 8H), 1.20 (t, 3H).

### EXAMPLE 57

### 3-{4-[(E)-3-(4-Cyclohexyl-piperazin-1-yl)-propenyl]-benzyl}-2-ethyl-5,7-dimethyl-3H-imidazo[4,5-b]pyridine dihydrochloride

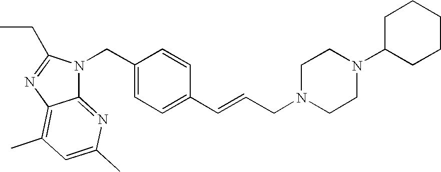

This compound was synthesized analogously to example 6 from alcohol 7b and 1-cyclohexylpiperazine.

MS (ESI): 472 [M+H]⁺, ¹H-NMR (DMSO-d6, 500 MHz) δ (ppm): 12.6-11.8 (br, 2H), 7.46 (d, 2H), 7.35 (s, 1H), 7.30 (d, 2H), 6.88 (d, 1H), 6.35 (dt, 1H), 5.69 (s, 2H), 3.94 (d, 2H), 3.80-3.25 (m, 9H), 3.20-3.00 (m, 2H), 2.66 (s, 3H), 2.58 (s, 3H), 2.09 (m, 2H), 1.79 (m, 2H), 1.57 (m, 1H), 1.43 (m, 2H), 1.27 (t, 3H) 1.10-0.90 (m, 2H).

### EXAMPLE 58

### [2-(4-{(E)-3-[4-(2-Ethyl-5,7-dimethyl-imidazo[4,5-b]pyridin-3-ylmethyl)-phenyl]-allyl}-piperazin-1-yl)-ethyl]-dimethyl-amine trihydrochloride

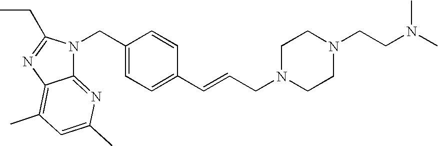

This compound was synthesized analogously to example 6 from alcohol 7b and 1-(2-dimetylaminoethyl)piperazine.

MS (ESI): 461 [M+H]⁺, ¹H-NMR (DMSO-d6, 500 MHz) δ (ppm): 12.0 (br, 2H), 10.65 (br, 1H), 7.48 (d, 2H), 7.38 (s, 1H), 7.32 (d, 2H), 6.85 (d, 1H), 6.41 (dt, 1H), 5.71 (s, 2H), 3.94 (d, 2H), 3.80-3.25 (m, 12H), 3.19 (q, 2H), 2.79 (s, 6H), 2.68 (s, 3H), 2.60 (s, 3H), 1.28 (t, 3H).

### EXAMPLE 59

### [3-(4-{(E)-3-[4-(2-Ethyl-5,7-dimethyl-imidazo[4,5-b]pyridin-3-ylmethyl)-phenyl]-allyl}-piperazin-1-yl)-propyl]-dimethyl-amine dihydrochloride

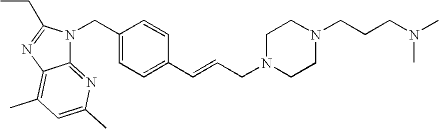

This compound was synthesized analogously to example 6 from alcohol 7b and dimethyl-(3-piperazin-1-yl-propyl)-amine.

MS (ESI): 475 [M+H]⁺, ¹H-NMR (DMSO-d₆): δ (ppm) 12.1 (br, 1H), 10.6 (br, 1H), 7.49 (d, 2H), 7.37 (s, 1H), 7.32 (d, 2H), 6.89 (d, 1H), 6.39 (dt, 1H), 5.7 (s, 2H), 3.1-4.6 (m, 16H), 2.77 (s, 3H), 2.76 (s, 3H), 2.67 (s, 3H), 2.60 (s, 3H), 2.15 (m, 2H), 1.29 (t, 3H).

### EXAMPLE 60

### Diethyl-[2-(4-{(E)-3-[4-(2-ethyl-5,7-dimethyl-imidazo[4,5-b]pyridin-3-ylmethyl)-phenyl]-allyl}-piperazin-1-yl)-ethyl]-amine hydrochloride

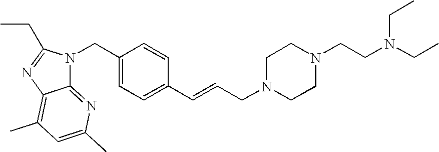

This compound was synthesized analogously to example 6 from alcohol 7b and diethyl-(2-piperazin-1-yl-ethyl)-amine.

MS (ESI): 489 [M+H]⁺, ¹H-NMR (121° C., DMSO-d₆): δ (ppm) 10.6 (br, 1H), 7.4 (d, 2H), 7.2 (d, 2H), 7.0 (s, 1H), 6.8 (d, 1H), 6.4 (dt, 1H), 5.5 (s, 2H), 3.8 (d, 2H), 2.8-3.3 (m, 18H), 2.6 (s, 3H), 2.5 (s, 3H), 1.3 (t, 9H).

### EXAMPLE 61

### [2-(4-{(E)-3-[4-(2-Ethyl-5,7-dimethyl-imidazo[4,5-b]pyridin-3-ylmethyl)-phenyl]-allyl}-piperazin-1-yl)-ethyl]-diisopropyl-amine trihydrochloride

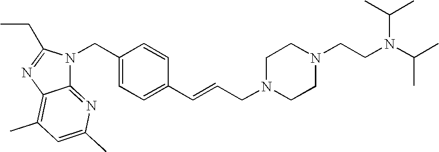

This compound was synthesized analogously to example 6 from alcohol 7b and 1-(2-diisopropyamino-ethyl)-piperazine.

MS (ESI): 461 [M+H]⁺, ¹H-NMR (DMSO-d6, 500 MHz) δ (ppm): 12.5-11.5 (br, 2H), 10.5-9.7 (br, 1H), 7.45 (d, 2H), 7.33 (s, 1H), 7.30 (d, 2H), 6.82 (d, 1H), 6.41 (dt, 1H), 5.67 (s, 2H), 4.20-3.00 (m, 18H), 2.64 (s, 3H), 2.58 (s, 3H), 1.32 (d, 12H), 1.26 (t, 3H).

### EXAMPLE 62

### 2-Ethyl-3-(4-{(E)-3-[4-(2-methoxy-ethyl)-piperazin-1-yl]-propenyl}-benzyl)-5,7-dimethyl-3H-imidazo[4,5-b]pyridine dihydrochloride

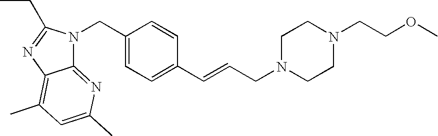

This compound was synthesized analogously to example 6 from alcohol 7b and 1-(2-methoxyethyl)-piperazine.

MS (ESI): 448 [M+H]⁺, ¹H-NMR (DMSO-d6, 500 MHz) δ (ppm): 12.5-11.5 (br, 2H), 7.45 (d, 2H), 7.29 (s, 1H), 7.28 (d, 2H), 6.85 (d, 1H), 6.36 (dt, 1H), 5.65 (s, 2H), 4.20-3.25 (m, 14H), 3.28 (s, 3H), 3.20.3.05 (m, 2H), 2.63 (s, 3H), 2.57 (s, 3H), 1.26 (t, 3H).

### EXAMPLE 63

### 3-(4-{(E)-3-[4-(2-Ethoxy-ethyl)-piperazin-1-yl]-propenyl}-benzyl)-2-ethyl-5,7-dimethyl-3H-imidazo[4,5-b]pyridine

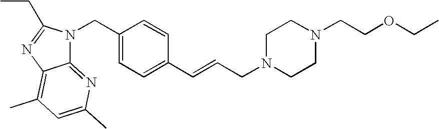

This compound was synthesized analogously to example 6 from alcohol 7b and 1-(2-ethoxy-ethyl)-piperazine without the formation of the hydrochloride salt.

MS (ESI): 462 [M+H]⁺, ¹H-NMR (DMSO-d₆): δ (ppm) 7.35 (d, 2H), 7.03 (d, 2H), 6.91 (s, 1H), 6.44 (d, 1H), 6.21 (dt, 1H), 5.40 (s, 2H), 3.41 (t, 2H), 3.36 (q, 2H), 3.01 (d, 2H), 2.73 (q, 2H), 2.49 (s, 3H), 2.48 (s, 3H), 2.41 (t, 2H), 2.36 (m, 8H), 1.20 (t, 3H), 1.07 (t, 3H).

### EXAMPLE 64

### 2-Ethyl-3-(4-{(E)-3-[4-(3-methoxy-propyl)-piperazin-1-yl]-propenyl}-benzyl)-5,7-dimethyl-3H-imidazo[4,5-b]pyridine dihydrochloride

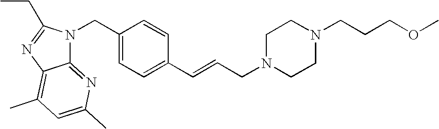

This compound was synthesized analogously to example 6 from alcohol 7b and 1-(3-methoxypropyl)-piperazine.

MS (ESI): 462 [M+H]⁺, ¹H-NMR (DMSO-d₆): δ (ppm) 12.0 (br, 1H), 10.2 (br, 1H), 7.40 (d, 2H), 7.17 (d, 2H), 6.96 (s, 1H), 6.69 (d, 1H), 6.27 (d, 1H), 5.47 (s, 2H), 3.5 (m, 2H), 3.44 (t, 2H), 3.29 (s, 3H), 3.0-3.3 (m, 10H), 2.85 (q, 2H), 2.56 (s, 3H), 2.55 (s, 3H), 1.85-1.95 (m, 2H), 1.3 (t, 3H).

### EXAMPLE 65

### 2-(4-{(E)-3-[4-(2-Ethyl-5,7-dimethyl-imidazo[4,5-b]pyridin-3-ylmethyl)-phenyl]-allyl}-piperazin-1-yl)-N,N-dimethyl-acetamide

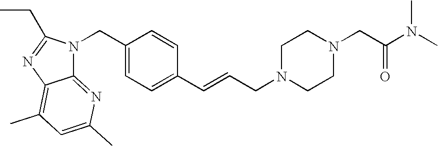

This compound was synthesized analogously to example 6 from alcohol 7b and N,N-dimethyl-2-piperazine-1-yl-acetamide without the formation of the hydrochloride salt.

MS (ESI): 475 [M+H]⁺, ¹H-NMR (DMSO-d6, 500 MHz) δ (ppm): 7.39 (d, 2H), 7.08 (d, 2H), 6.94 (s, 1H), 6.80-6.55 (m, 1H), 6.35-6.15 (m, 1H), 5.43 (s, 2H), 2.95 (s, 3H), 2.81 (s, 3H), 2.90-2.60 (m, 14H), 2.50 (s, 6H), 1.20 (t, 3H).

### EXAMPLE 66

### 2-Ethyl-5,7-dimethyl-3-(4-{(E)-3-[4-(2-morpholin-4-yl-ethyl)-piperazin-1-yl]-propenyl}-benzyl)-3H-imidazo[4,5-b]pyridine trihydrochloride

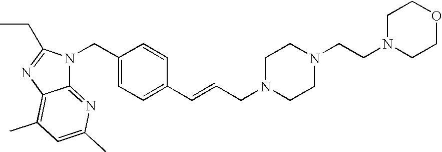

This compound was synthesized analogously to example 6 from alcohol 7b and 1-(2-(4-morpholino)-ethyl)-piperazine.

MS (ESI): 503 [M+H]⁺, ¹H-NMR (DMSO-d6, 500 MHz) δ (ppm): 12.0-11.0 (br, 2H), 9.3 (br s, 1H), 7.45 (d, 2H), 7.34 (s, 1H), 7.29 (d, 2H), 6.81 (d, 1H), 6.40 (dt, 1H), 5.67 (s, 2H), 4.00-3.90 (m, 4H), 3.85-3.05 (m, 20H), 2.65 (s, 3H), 2.58 (s, 3H), 1.29 (t, 3H).

### EXAMPLE 67

### 3-(4-{(E)-3-[4-(3,4-Dimethoxy-benzyl)-piperazin-1-yl]-propenyl}-benzyl)-2-ethyl-5,7-dimethyl-3H-imidazo[4,5-b]pyridine trihydrochloride

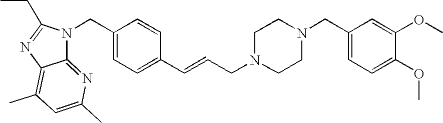

This compound was synthesized analogously to example 6 from alcohol 7b and 1-(3,4-dimethoxybenzyl)piperazine.

MS (ESI): 540 [M+H]⁺, ¹H-NMR (DMSO-d6, 500 MHz) δ (ppm): 12.3 (br, 2H), 7.45 (d, 2H), 7.44 (s, 1H), 7.32 (s, 1H), 7.29 (d, 2H), 7.10 (d, 1H), 6.98 (d, 1H), 6.85 (d, 1H), 6.37 (m, 1H), 5.66 (s, 2H), 4.30 (s, br, 2H), 4.00-3.90 (m, 2H), 3.78 (s, 3H), 3.77 (s, 3H), 3.70-3.05 (m, 10H), 2.64 (s, 3H), 2.57 (s, 3H), 1.26 (t, 3H).

### EXAMPLE 68

### 2-Ethyl-3-(4-{(E)-3-[4-(3-fluoro-propyl)-piperazin-1-yl]-propenyl}-benzyl)-5,7-dimethyl-3H-imidazo[4,5-b]pyridine

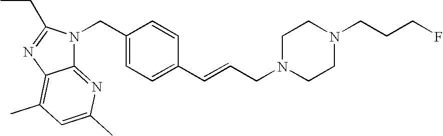

This compound was synthesized analogously to example 6 from alcohol 7b and 3-fluoropropan-1-ol without the formation of the hydrochloride salt.

MS (ESI): 450 [M+H]⁺, ¹H-NMR (DMSO-d6, 500 MHz) δ (ppm): 7.36 (d, 2H), 7.03 (d, 2H), 6.93 (s, 1H), 6.45 (d, 1H), 6.23 (dt, 1H), 5.41 (s, 2H), 4.49 (q, 1H), 4.39 (q, 1H), 3.03 (d, 2H), 2.76 (q, 2H), 2.49 (s, 6H), 2.45-2.20 (m, 10H), 1.85-1-70 (m, 2H), 1.20 (t, 3H).

### EXAMPLE 69

### 2-Ethyl-3-{4-[(E)-3-(4ethyl-3-methyl-piperazin-1-yl)-propenyl]-benzyl}-5,7-dimethyl-3H-imidazo[4,5-b]pyridine dihydrochloride

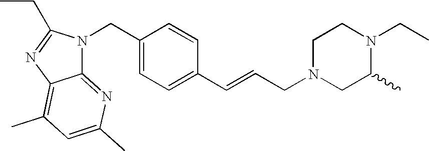

This compound was synthesized analogously to example 6 from alcohol 7b and 1-ethyl-2-methyl-piperazine.

MS (ESI): 432 [M+H]⁺, ¹H-NMR (DMSO-d6, 500 MHz) δ (ppm): 12.5-11.8 (br, 2H), 7.45 (d, 2H), 7.29 (s, 1H), 7.28 (d, 2H), 6.87 (d, 1H), 6.39 (m, 1H), 5.65 (s, 2H), 4.30-3.25 (m, 9H), 3.20-3.05 (m, 4H), 2.62 (s, 3H), 2.57 (s, 3H), 1.37 (d, 3H), 1.26 (t, 3H), 1.21 (t, 3H).

### EXAMPLE 70

### 2-Ethyl-3-{4-[(E)-3-((S)-2-isopropyl-4-methyl-piperazin-1-yl)-propenyl]-benzyl}-5,7-dimethyl-3H-imidazo[4,5-b]pyridine dihydrochloride

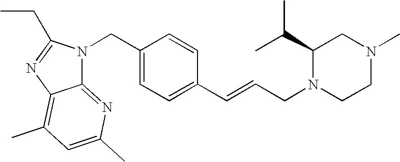

This compound was synthesized analogously to example 6 from alcohol 7b and (S)-3-isopropyl-1-methylpiperazine.

MS (ESI): 446 [M+H]⁺, ¹H-NMR (DMSO-d₆): δ (ppm) 12 (br, 1H), 10-9.5 (br, 1H), 7.36 (d, 2H), 7.17 (d, 2H), 6.98 (s, 1H), 6.62 (d, 1H), 6.26 (dt, 1H), 5.47 (s, 2H), 3.5-3.0 (m, 9H), 2.86 (q, 2H), 2.68 (s, 3H), 2.56 (s, 3H), 2.54 (s, 3H), 2.04 (m, 1H), 1.29 (t, 3H), 1.06 (d, 3H), 1.03 (d, 3H).

### EXAMPLE 71

### 2-Ethyl-3-{4-[(E)-3-(4-isopropyl-3-methyl-piperazin-1-yl)-propenyl]-benzyl}-5,7-dimethyl-3H-imidazo[4,5-b]pyridine

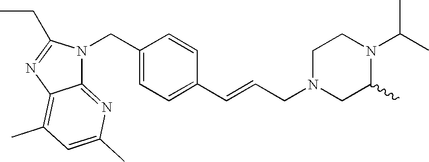

This compound was synthesized analogously to example 6 from alcohol 7b and 1-isopropyl-2-methylpiperazine without the formation of the hydrochloride salt.

MS (ESI): 450 [M+H]⁺, ¹H-NMR (DMSO-d6, 500 MHz) δ (ppm): 7.36 (d, 2H), 7.03 (d, 2H), 6.93 (s, 1H), 6.44 (d, 1H), 6.23 (dt 1H), 5.41 (s, 2H), 3.12 (m, 1H), 2.99 (m, 2H), 2.74 (q, 2H), 2.70-2.55 (m, 1H), 2.49 (s, 6H), 2.40-1.70 (m, 6H), 1.21 (t, 3H), 1.00 (d, 3H), 0.90 (d, 3H), 0.80 (d, 3H).

### EXAMPLE 72

### 2-Ethyl-3-{4-[(E)-3-(3-ethyl-4-isopropyl-piperazin-1-yl)-propenyl]-benzyl}-5,7-dimethyl-3H-imidazo[4,5-b]pyridine dihydrochloride

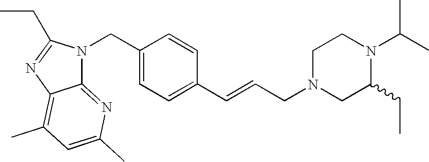

This compound was synthesized analogously to example 6 from alcohol 7b and 2-ethyl-1-isopropylpiperazine.

MS (ESI): 460 [M+H]⁺, ¹H-NMR (DMSO-d₆): δ (ppm) 12.6-11.6 (br, 2H), 7.47 (d, 2H), 7.30 (s, 1H), 7.29 (d, 2H), 6.89 (d, 1H), 6.38 (dt, 1H), 5.66 (s, 2H), 4.0-2.9 (m, 12H), 2.63 (s, 3H), 2.58 (s, 3H), 2.1-1.6 (m, 2H), 1.38 (m, 3H), 1.27 (t, 3H), 1.13 (m, 3H). 0.95 (t, 3H).

### EXAMPLE 73

### 2-Ethyl-5,7-dimethyl-3-{4-[(E)-3-(1-methyl-hexahydro-pyrrolo[1,2-a]pyrazin-2-yl)-propenyl]-benzyl}-3H-imidazo[4,5-b]pyridine dihydrochloride

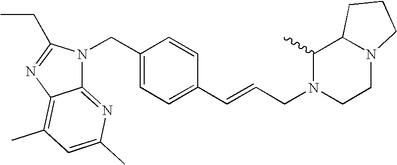

This compound was synthesized analogously to example 6 from alcohol 7b and 1-methyloctahydropyrrolo[1,2-a]pyrazine.

MS (ESI): 444 [M+H]⁺, ¹H-NMR (DMSO-d6, 500 MHz) δ (ppm): 13-12.1 (br, 2H), 7.50 (d, 2H), 7.30 (s, 1H), 7.28 (d, 2H), 6.9 (d, 1H), 6.4 (m, 1H), 5.7 (s, 2H), 4.25-2.95 (m, 12H), 2.62 (s, 3H), 2.57 (s, 3H), 2.3-1.5 (m, 4H), 1.4 (d, 3H), 1.3 (t, 3H).

### EXAMPLE 74

### 2-Ethyl-3-{4-[(E)-3-((1R,4R)-5-ethyl-2,5-diaza-bicyclo[2.2.1]hept-2-yl)-propenyl]-benzyl}-5,7-dimethyl-3H-imidazo[4,5-b]pyridine

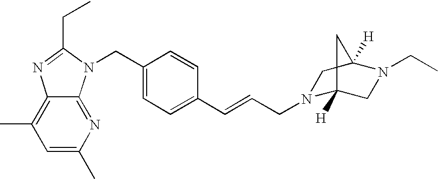

This compound was synthesized analogously to example 6 from alcohol 7b and (1R,4R)-2-ethyl-2,5-diazabicyclo[2.2.1]heptane without the formation of the hydrochloride salt.

MS (ESI): 430 [M+H]⁺, ¹H-NMR (DMSO-d6, 500 MHz) δ (ppm): 7.36 (d, 2H), 7.03 (d, 2H), 6.93 (s, 1H), 6.42 (d, 1H), 6.18 (dt,1H), 5.41 (s, 2H), 3.3-3.1 (m, 4H), 2.76 (q, 2H), 2.65-2.5 (m, 6H), 2.49 (s, 6H), 1.50 (m, 2H), 1.2 (t, 3H), 0.90 (t, 3H).

### EXAMPLE 75

### 2-Ethyl-3-{4-[(E)-3-((1R,4R)-5-isobutyl-2,5-diaza-bicyclo[2.2.1]hept-2-yl)-propenyl]-benzyl}-5,7-dimethyl-3H-imidazo[4,5-b]pyridine dihydrochloride

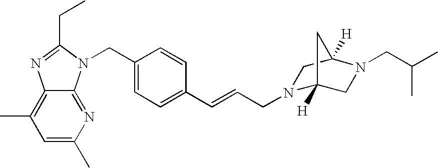

This compound was synthesized analogously to example 6 from alcohol 7b and (1R,4R)-2-isobutyl-2,5-diazabicyclo[2.2.1]heptane.

MS (ESI): 458 [M+H]⁺, ¹H-NMR (DMSO-d6, 500 MHz) δ (ppm): 12.6-11.0 (br, 2H), 7.45 (d, 2H), 7.4 (s, 1H), 7.28 (d, 2H), 6.8 (d, 1H), 6.4 (dt, 1H), 5.7 (s, 2H), 4.5 (m, 2H), 4.2-3.3 (m, 8H), 3.20 (q, 2H), 2.62 (s, 3H), 2.57 (s, 3H), 2.0 (m, 1H), 1.5 (d, 2H), 1.26 (t, 3H), 1.00-0.90 (m, 6H).

### EXAMPLE 76

### 3-{4-[(E)-3-((1R,4R)-5-Cyclopropylmethyl-2,5-diaza-bicyclo[2.2.1]hept-2-yl)-propenyl]-benzyl}-2-ethyl-5,7-dimethyl-3H-imidazo[4,5-b]pyridine trihydrochloride

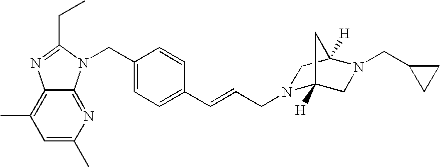

This compound was synthesized analogously to example 6 from alcohol 7b and (1R,4R)-2-(cyclopropylmethyl)-2,5-diazabicyclo[2.2.1]heptane without the formation of the hydrochloride salt.

MS (ESI): 456 [M+H]⁺, ¹H-NMR (DMSO-d6, 500 MHz) δ (ppm): 7.36 (d, 2H), 7.03 (d, 2H), 6.93 (s, 1H), 6.49 (d, 1H), 6.20 (dt, 1H), 5.41 (s, 2H), 3.5-3.1 (m, 6H), 2.76 (q, 2H), 2.7-2.5 (m, 2H), 2.49 (s, 6H), 2.40-2.30 (m, 2H), 1.60-1.50 (m, 2H), 1.21 (t, 3H), 0.80-0.70 (m, 1H), 0.45 (m, 2H), 0.1-0 (m, 2H).

### EXAMPLE 77

### (4-{(E)-3-[4-(2-Ethyl-5,7-dimethyl-imidazo[4,5-b]pyridin-3-ylmethyl)-phenyl]-allyl}-morpholin-2-ylmethyl)-carbamic acid tert-butyl ester

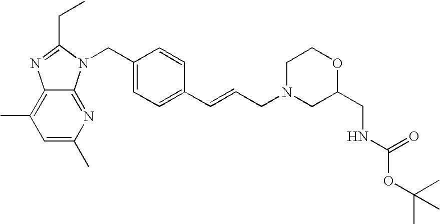

This compound was synthesized analogously to example 6 from alcohol 7b and morpholin-2-ylmethyl-carbamic acid tert-butyl ester without the formation of the hydrochloride salt.

MS (ESI): 520 [M+H]⁺, ¹H-NMR (DMSO-d6, 500 MHz) δ (ppm): 7.37 (d, 2H), 7.06 (d, 2H), 6.94 (s, 1H), 6.81 (t, 1H), 6.47 (d, 1H), 6.23 (dt, 1H), 5.42 (s, 2H), 3.73 (m, 1H), 3.5-3.45 (m, 2H), 3.15-2.8 (m, 4H), 2.76 (q, 2H), 2.8-2.7 (m, 1H), 2.7-2.6 (m, 1H), 2.49 (s, 6H), 2.05-1.95 (m, 1H), 1.70-1.60 (m, 1H), 1.29 (s, 9H), 1.21 (t, 3H).

### EXAMPLE 78

### C-(4-{(E)-3-[4-(2-Ethyl-5,7-dimethyl-imidazo[4,5-b]pyridin-3-ylmethyl)-phenyl]-allyl}-morpholin-2-yl)-methylamine dihydrochloride

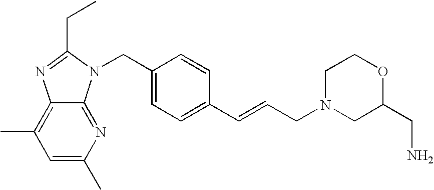

(4-{(E)-3-[4-(2-Ethyl-5,7-dimethyl-imidazo[4,5-b]pyridin-3-ylmethyl)-phenyl]-allyl}-morpholin-2-ylmethyl)-carbamic acid tert-butyl ester (example 77) (528 mg, 1.016 mmol) was dissolved in dioxane (5 ml) and after addition of 4N HCl in dioxane (1.016 ml, 4.06 mmol) the mixture was stirred for 3 h at rt. Then the mixture was evaporated under reduced pressure and dried at high vacuum to give a yellow solid.

MS (ESI): 420 [M+H]⁺, ¹H-NMR (DMSO-d6, 500 MHz) δ (ppm): 8.13 (br, 3H), 7.45 (d, 2H), 7.28 (s, 1H), 7.27 (d, 2H), 6.79 (d, 1H), 6.40 (dt, 1H), 5.63 (s, 2H), 4.25-3.75 (m, 5H), 3.50-3.3 (m, 2H), 3.1-2.95 (m, 4H), 2.9-2.7 (m, 2H), 2.62 (s, 3H), 2.56 (s, 3H), 1.25 (t, 3H).

### EXAMPLE 79

### (4-{(E)-3-[4-(2-Ethyl-5,7-dimethyl-imidazo[4,5-b]pyridin-3-ylmethyl)-phenyl]-allyl}-morpholin-2-ylmethyl)-isopropyl-amine

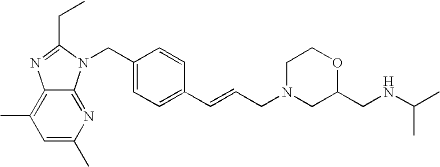

To a mixture of C-(4-{(E)-3-[4-(2-ethyl-5,7-dimethyl-imidazo[4,5-b]pyridin-3-ylmethyl)-phenyl]-allyl}-morpholin-2-yl)-methylamine (example 78) (100 mg, 0.203 mmol) and acetone (0.022 ml, 0.305 mmol) in CH₂Cl₂ (2 ml) was added sodium triacetoxyborohydride (86 mg, 0.406 mmol). The mixture was stirred for 16 h at rt. The reaction mixture was extracted with 1M NaOH. The combined organic layers were dried over Na₂SO₄ and evaporated. The crude product was purified by preparative HPLC. The hydrochloride was prepared by adding excess methanolic HCl to the pure product followed by evaporation.

MS (ESI): 462 [M+H]⁺, ¹H-NMR (DMSO-d6+NaOD, 500 MHz) δ (ppm): 7.36 (d, 2H), 7.03 (d, 2H), 6.93 (s, 1H), 6.55-6.40 (m, 1H), 6.30-6.15 (m, 1H), 5.41 (s, 2H), 3.85-3.40 (m, 3H), 3.10-3.00 (m, 2H), 2.76 (q, 2H), 2.80-2.50 (m, 6H), 2.49 (s, 6H), 2.05-1.95 (m, 1H), 1.80-1.70 (m, 1H), 1.19 (t, 3H), 1.00-0.90 (m, 6H).

### EXAMPLE 80

### 2-Ethyl-3-[4-((E)-3-imidazol-1-yl-propenyl)-benzyl]-5,7-dimethyl-3H-imidazo[4,5-b]pyridine hydrochloride

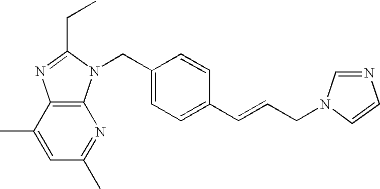

A mixture of 3-(4-bromo-benzyl)-2-ethyl-5,7-dimethyl-3H-imidazo[4,5-b]pyridine (5h) (100 mg, 0.290 mmol), 1-allylimidazole (0.031 ml, 0.290 mmol), di-cyclohexyl-methylamine (0.123 ml 0.581 mmol) and Pd(PtBu₃)₂ (2.97 mg, 0.0058 mmol) in dioxane (2 ml) was stirred for 10 min at 130° C. in the microwave oven. The reaction mixture was extracted with EtOAc/saturated aqueous NaHCO₃ solution. The combined organic layers were dried over Na₂SO₄ and evaporated. The crude product was purified by preparative HPLC. The hydrochloride was prepared by adding excess methanolic HCl to the pure product followed by evaporation.

MS (ESI): 372 [M+H]⁺, ¹H-NMR (DMSO-d6, 500 MHz) δ (ppm): 9.20 (s, 1H), 7.85-7.65 (m, 2H), 7.45 (d, 2H), 7.29 (s, 1H), 7.28 (d, 2H), 6.75-6.65 (m, 1H), 6.55-6.40 (m, 1H), 5.63 (s, 2H), 4.98 (d, 2H), 3.15-3.00 (m, 2H), 2.62 (s, 3H), 2.57 (s, 3H), 1.25 (t, 3H).

### EXAMPLE 81

### 3-{4-[(E)-3-(4-Isopropyl-piperazin-1-yl)-propenyl]-benzyl}-5,7-dimethyl-2-propyl-3H-imidazo[4,5-b]pyridine dihydrochloride

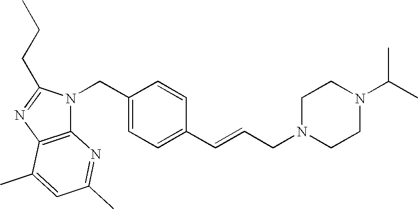

This compound was synthesized analogously to example 6 from alcohol 7c and 1-isopropyl-piperazine.

MS (ESI): 446 [M+H]⁺, ¹H-NMR (DMSO-d₆): δ (ppm) 12.1 (br, 2H), 7.47 (d, 2H), 7.30 (s, 1H), 7.29 (d, 2H), 6.89 (d, 1H), 6.37 (dt, 1H), 5.67 (s, 2H), 3.0-4.0 (m, 13H), 2.64 (s, 3H), 2.59 (s, 3H), 1.72 (m, 2H), 1.3 (d, 6H), 0.92 (t, 3H).

### EXAMPLE 82

### 1′-{(E)-3-[4-(5,7-Dimethyl-2-propyl-imidazo[4,5-b]pyridin-3-ylmethyl)-phenyl]-allyl}-[1,4′]bipiperidine dihydrochloride

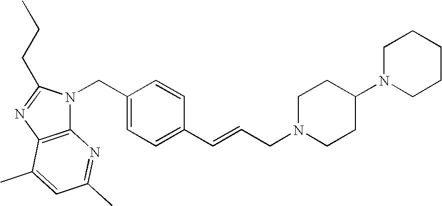

This compound was synthesized analogously to example 6 from alcohol 7c and [1,4′]bipiperidinyl.

MS (ESI): 486 [M+H]⁺, ¹H-NMR (DMSO-d₆): δ (ppm) 11.0 (br, 1H), 10.7 (br, 1H), 7.47 (d, 2H), 7.24 (d, 2H), 7.17 (br, 1H), 6.80 (d, 1H), 6.37 (dt, 1H), 5.60 (s, 2H), 2.8-3.9 (m, 13H), 2.62 (s, 3H), 2.58 (s, 3H), 1.75-2.4 (m, 8H), 1.7 (m, 2H), 1.4 (m, 2H), 0.92 (t, 3H).

### EXAMPLE 83

### 1′-{(E)-3-[4-(2-Isopropyl-5,7-dimethyl-imidazo[4,5-b]pyridin-3-ylmethyl)-phenyl]-allyl}-[1,4′]bipiperidinyl dihydrochloride

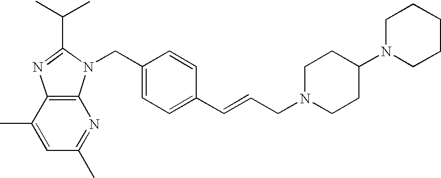

This compound was synthesized analogously to example 6 from alcohol 7d and 4-piperidino-piperidine.

MS (ESI): 486 [M+H]⁺, ¹H-NMR (DMSO-d₆): δ (ppm) 11.15 (br, 1H), 10.7 (br, 1H), 7.46 (d, 2H), 7.28 (s, 1H), 7.24 (d, 2H), 6.77 (d, 1H), 6.40 (dt, 1H), 5.69 (s, 2H), 3.82 (m, 2H), 3.4-3.7 (m, 3H), 3.2-3.4 (m, 3H), 2.97 (dd, 2H), 2.89 (dd, 2H), 2.86 (s, 3H), 2.58 (s, 3H), 2.1-2.4 (m, 4H), 1.35-2.0 (m, 6H), 1.34 (d, 6H).

### EXAMPLE 84

### 2-Cyclopropyl-3-{4-[(E)-3-(4-isopropyl-piperazin-1-yl)-propenyl]-benzyl}-5,7-dimethyl-3H-imidazo[4,5-b]pyridine dihydrochloride

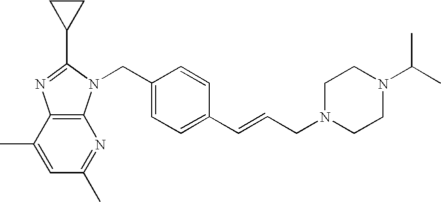

This compound was synthesized analogously to example 6 from alcohol 7e and 1-isopropylpiperazine.

MS (ESI): 444 [M+H]⁺, ¹H-NMR (DMSO-d6, 500 MHz) δ (ppm): 12.5-11.7 (br, 2H), 7.45 (d, 2H), 7.29 (d, 2H), 7.22 (s, 1H), 6.6.87 (d, 1H), 6.38 (dt, 1H), 5.71 (s, 2H), 4.10-3.40 (m, 11H), 2.58 (s, 3H), 2.55 (s, 3H), 1.75-1-60 (m, 1H), 1.45-1.15 (m, 4H) 1.27 (d, 6H).

### EXAMPLE 85

### 2-Butyl-3-{4-[(E)-3-(4-isopropyl-piperazin-1-yl)-propenyl]-benzyl}-5,7-dimethyl-3H-imidazo[4,5-b]pyridine dihydrochloride

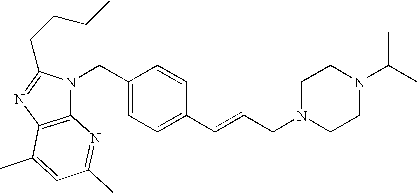

This compound was synthesized analogously to example 6 from alcohol 7f and 1-isopropyl-piperazine.

MS (ESI): 460 [M+H]⁺, ¹H-NMR (DMSO-d₆): δ (ppm) 11.8 (br, 2H), 7.45 (d, 2H), 7.21 (d, 2H), 7.11 (br s, 1H), 6.83 (br, 1H), 6.33 (m, 1H), 5.56 (s, 2H), 3.0-4.0 (m, 11H), 2.9 (m, 2H), 2.55 (s, 3H), 2.54 (s, 3H), 1.64 (m, 2H), 1.34 (m, 2H), 1.29 (d, 6H), 0.85 (t, 3H).

### EXAMPLE 86

### 1′-{(E)-3-[4-(2-Butyl-5,7-dimethyl-imidazo[4,5-b]pyridin-3-ylmethyl)-phenyl]-allyl}-[1,4′]bipiperidine dihydrochloride

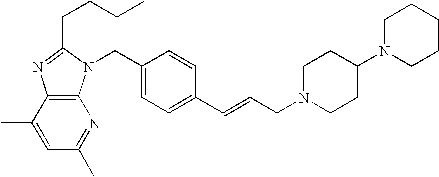

This compound was synthesized analogously to example 6 from alcohol 7f and 4-piperidino-piperidine.

MS (ESI): 500 [M+H]⁺, ¹H-NMR (DMSO-d₆): δ (ppm) 11.2 (br, 1H), 10.85 (br, 1H), 7.47 (d, 2H), 7.30 (s, 1H), 7.29 (d, 2H), 6.80 (d, 1H), 6.43 (dt, 1H), 5.68 (s, 2H), 3.84 (m, 2H), 2.75-3.8 (m, 10H), 2.64 (s, 3H), 2.59 (s, 3H), 1.3-2.5 (m, 15H), 0.84 (t, 3H).

### EXAMPLE 87

### 1′-{(E)-3-[4-(5,7-Dimethyl-2-trifluoromethyl-imidazo[4,5-b]pyridin-3-ylmethyl)-phenyl]-allyl}-[1,4′]bipiperidinyl dihydrochloride

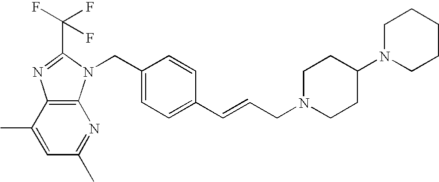

This compound was synthesized analogously to example 6 from alcohol 7g and 4-piperidino-piperidine.

MS (ESI): 512 [M+H]⁺, ¹H-NMR (DMSO-d6, 500 MHz) δ (ppm): 10.8 (br. S, 1H), 10.5 (br. S, 1H), 7.35 (d, 2H), 7.23 (s, 1H), 7.05 (d, 2H), 6.43 (d, 1H), 6.25 (dt, 1H), 5.61 (s, 2H), 3.01 (d, 2H), 2.90-2.80 (m, 2H), 2.59 (s, 3H), 2.57 (s, 3H), 2.50-2.35 (m, 4H), 2.20-2.05 (m, 1H), 1.90-1.80 (m, 2H), 1.70-1.55 (m, 2H), 1.5-1.25 (m, 8H).

### EXAMPLE 88

### 5,7-Dimethyl-3-(4-{(E)-3-[4-(4-methyl-piperazin-1-yl)-piperidin-1-yl]-propenyl}-benzyl)-2-trifluoromethyl-3H-imidazo[4,5-b]pyridine trihydrochloride

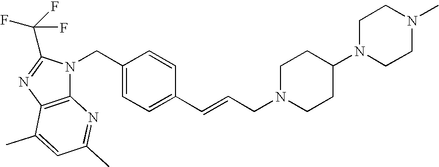

This compound was synthesized analogously to example 6 from alcohol 7g and 1-methyl-4-(piperidin-4-yl)piperazine.

MS (ESI): 527 [M+H]⁺, ¹H-NMR (DMSO-d6, 500 MHz) δ (ppm): 11.8 (br, 2H), 10.80 (br, 1H), 7.45 (d, 2H), 7.24 (s, 1H), 7.13 (d, 2H), 6.78 (d, 1H), 6.35 (dt, 1H), 5.64 (s, 3H), 3.84 (br, 2H), 3.20-3.75 (m, 10H), 2.95 (m, 2H), 2.81 (s, 3H9, 2.61 (s, 3H), 2.58 (s, 3H), 2.26 (br m, 2H), 2.03 (br. M, 2H).

### EXAMPLE 89

### 5,7-Dimethyl-3-{4-[(E)-3-(4-morpholin-4-yl-piperidin-1-yl)-propenyl]-benzyl}-2-trifluoromethyl-3H-imidazo[4,5-b]pyridine dihydrochloride

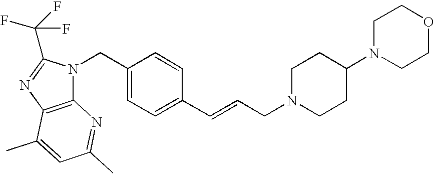

This compound was synthesized analogously to example 6 from alcohol 7g and 4-morpholinopiperidine.

MS (ESI): 514 [M+H]⁺, ¹H-NMR (DMSO-d6, 500 MHz) δ (ppm): 11.2 (br, 1H), 10.4 (br, 1H), 7.44 (d, 2H), 7.24 (s, 1H), 7.14 (d, 2H), 6.78 (d, 1H), 6.33 (dt, 1H), 5.65 (s, 2H), 4.0-3.85 (m, 2H), 3.85-3.70 (m, 4H), 3.6-3.55 (m, 2H), 3.45-3.3 (m, 3H), 3.1-2.95 (m, 2H), 2.95-2.85 (m, 2H), 2.60 (s, 3H), 2.57 (s, 3H), 2.35-2.25 (m, 2H), 2.05-1.9 (m, 2H).

### EXAMPLE 90

### 1-{(E)-3-[4-(5,7-Dimethyl-2-trifluoromethyl-imidazo[4,5-b]pyridin-3-ylmethyl)-phenyl]-allyl}-piperidine-4-carboxylic acid methyl ester hydrochloride

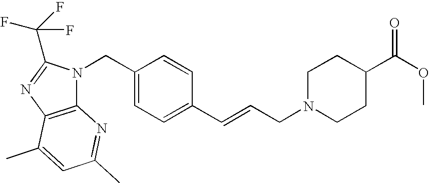

This compound was synthesized analogously to example 6 from alcohol 7g and methyl piperidine-4-carboxylate.

MS (ESI): 487 [M+H]⁺, ¹H-NMR (DMSO-d6, 500 MHz) δ (ppm): 10.45 (br d, 1H), 7.45 (d, 2H), 7.25 (s, 1H), 7.14 (d, 2H), 6.78 (d, 1H), 6.33 (m, 1H), 5.65 (s, 2H), 3.82 (m, 2H), 3.63 (s, 3H), 3.45 (m, 2H), 2.95 (m, 2H), 2.61 (s, 3H), 2.58 (s, 3H), 2.05 (m, 2H), 1.8 (m, 2H).

### EXAMPLE 91

### 3-{4-[(E)-3-(4-Isopropyl-piperazin-1-yl)-propenyl]-benzyl}-5,7-dimethyl-2-trifluoromethyl-3H-imidazo[4,5-b]pyridine dihydrochloride

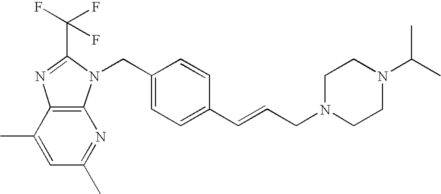

This compound was synthesized analogously to example 6 from alcohol 7g and 1-isopropylpiperazine.

MS (ESI): 472 [M+H]⁺, ¹H-NMR (DMSO-d6, 500 MHz, as free base) δ (ppm): 7.36 (d, 2H), 7.23 (s, 1H), 7.05 (d, 2H), 6.45 (d, 1H), 6.30-6.15 (m, 1H), 5.61 (s, 2H), 3.01 (d, 2H), 2.59 (s, 3H), 2.57 (s, 3H), 2.60-2.50 (m, 1H), 2.45-2.20 (m, 8H), 0.94 (d, 6H).

### EXAMPLE 92

### 3-{4-[(E)-3-(4-tert-Butyl-piperazin-1-yl)-propenyl]-benzyl}-5,7-dimethyl-2-trifluoromethyl-3H-imidazo[4,5-b]pyridine

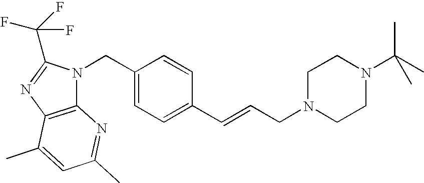

This compound was synthesized analogously to example 6 from alcohol 7g and 1-t-butylpiperazine without the formation of the hydrochloride salt.

MS (ESI): 486 [M+H]⁺, ¹H-NMR (DMSO-d6, 500 MHz) δ (ppm): 7.36 (d, 2H), 7.23 (s, 1H), 7.05 (d, 2H), 6.45 (d, 1H), 6.30-6.15 (m, 1H), 5.61 (s, 2H), 3.01 (d, 2H), 2.59 (s, 3H), 2.57 (s, 3H), 2.45-2.20 (m, 8H), 0.97 (s, 9H).

### EXAMPLE 93

### 1′-{(Z)-3-[4-(2-Ethyl-5,7-dimethyl-imidazo[4,5-b]pyridin-3-ylmethyl)-phenyl]-2-fluoro-allyl}-[1,4′]bipiperidinyl dihydrochloride

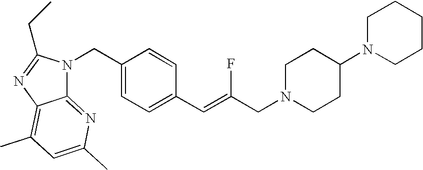

This compound was synthesized analogously to example 6 from alcohol 7h and 4-piperidino-piperidine.

MS (ESI): 490 [M+H]⁺, ¹H-NMR (DMSO-d6+NaOD, 500 MHz) δ (ppm): 7.40 (d, 2H), 7.08 (d, 2H), 6.94 (s, 1H), 5.80 (d, br, 1H), 5.42 (s, 2H), 3.13 (d, 2H), 2.92-2.85 (m, 2H), 2.74 (q, 2H), 2.50 (s, 6H), 2.43-2.37 (m, 4H), 2.16-2.09 (m, 1H), 2.05-1.93 (m, 2H), 1.70-1.30 (m, 10H), 1.20 (t, 3H).

### EXAMPLE 94

### 2-Ethyl-3-{4-[(Z)-2-fluoro-3-(4-isopropyl-piperazin-1-yl)-propenyl]-benzyl}-5,7-dimethyl-3H-imidazo[4,5-b]pyridine dihydrochloride

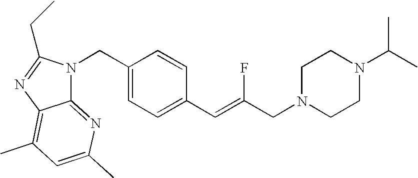

This compound was synthesized analogously to example 6 from alcohol 7h and 1-isopropylpiperazine.

MS (ESI): 450 [M+H]⁺, ¹H-NMR (DMSO-d6, 500 MHz) δ (ppm): 7.48 (d, 2H), 7.31 (s, 1H), 7.28 (d, 2H), 6.13 (d, br, 1H), 5.65 (s, 2H), 3.50-3.43 (m, 3H), 3.39-3.16 (m, 8H), 3.13-3.08 (br q, 2H), 2.63 (s, 3H), 2.58 (s, 3H), 1.27 (d, 6H), 1.26 (t, 3H).

### EXAMPLE 95

### 3-{4-[(Z)-3-(4-tert-Butyl-piperazin-1-yl)-2-fluoro-propenyl]-benzyl}-2-ethyl-5,7-dimethyl-3H-imidazo[4,5-b]pyridine dihydrochloride

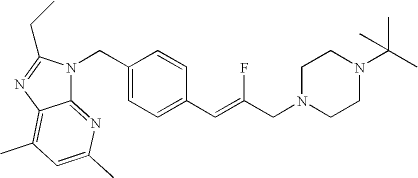

This compound was synthesized analogously to example 6 from alcohol 7h and 1-t-butylpiperazine.

MS (ESI): 464 [M+H]⁺, ¹H-NMR (DMSO-d6, 500 MHz) δ (ppm): 7.48 (d, 2H), 7.34 (s, 1H), 7.32 (d, 2H), 6.24 (d, br, 1H), 5.68 (s, 2H), 3.65-3.55 (m, 2H), 3.50-3.29 (m, 8H), 3.13 (q, 2H), 2.65 (s, 3H), 2.59 (s, 3H), 1.36 (s, 9H), 1.27 (t, 3H).

Synthesis of the cinnamide Derivatives

The cinnamide Derivatives were Generally Prepared by a Synthesis as Lined Out in Reaction Scheme 15.

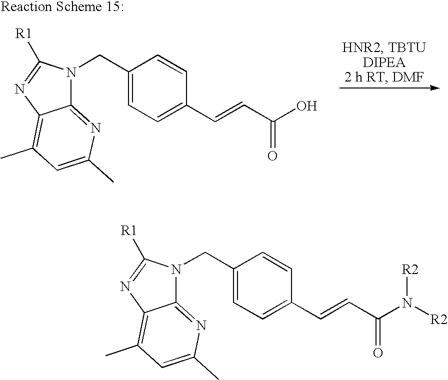

### EXAMPLE 96

### (E)-1-(4-Diethylamino-piperidin-1-yl)-3-[4-(2-ethyl-5,7-dimethyl-imidazo[4,5-b]pyridin-3-ylmethyl)-phenyl]-propenone

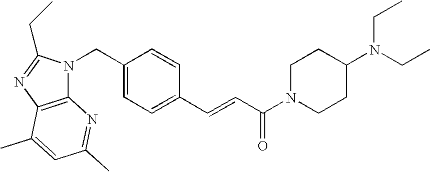

(E)-3-[4-(2-ethyl-5,7-dimethyl-imidazo[4,5-b]pyridin-3-ylmethyl)-phenyl]-acrylic acid (9b) (100 mg, 0.3 mmol) was dissolved in 4 ml of DMF and after addition of diethyl-piperidin-4-yl-amine (46.6 mg, 0.3 mmol), TBTU (109 mg, 0.33 mmol) and N-ethyldiisopropylamin (193 μl, 1.19 mmol) the mixture was stirred for 2 h (TLC control) at room temperature. Then the mixture was evaporated under reduced pressure. The residue was diluted with ethyl acetate, washed with sat. NaHCO₃— and NaCl-solution, and dried over Na₂SO₄. Evaporation gives a brown oil. The crude product was purified by flash-chromatography (methanol/ethyl acetate (1:3), silicagel), which yields a yellow solid.

MS (ESI): 474 [M+H]⁺, ¹H-NMR (DMSO-d₆): δ (ppm) 7.65 (d, 2H), 7.40 (d, 1H), 7.23 (d, 1H), 7.16 (d, 2H), 6.96 (s, 1H), 5.48 (s, 2H), 4.48 (m, 1H), 4.26 (m, 1H), 3.03 (m, 1H), 2.79 (q, 2H), 2.73 (m, 1H), 2.61 (m, 1H), 2.52 (s, 3H), 2.51 (s, 3H), 2.47 (q, 4H), 1.70 (m, 2H), 1.30 (m, 2H), 1.23 (t, 3H), 0.96 (t, 6H).

### EXAMPLE 97

### (E)-1-[1,4′]Bipiperidinyl-1′-yl-3-[4-(2-ethyl-5,7-dimethyl-imidazo[4,5-b]pyridin-3-ylmethyl)-phenyl]-propenone hydrochloride

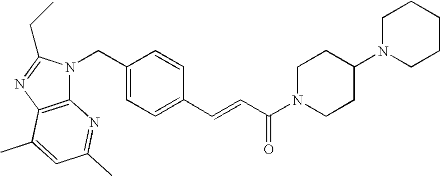

This compound was synthesized analogously to example 23 from acid (6) and 4-piperidino-piperidine. The HCl salt was prepared by adding excess methanolic HCl to the pure product followed by evaporation.

MS (ESI): 486 [M+H]⁺, ¹H-NMR (DMSO-d₆): δ (ppm) 10.51 (br, 1H), 7.58 (d, 2H), 7.41 (d, 1H), 7.23 (d, 2H), 7.07 (d, 1H), 7.03 (s, 1H), 5.53 (s, 2H), 4.45 (d, 2H), 3.38 (m, 3H), 2.89-2.95 (m, 6H), 2.58 (s, 3H), 2.55 (s, 3H), 2.20 (m, 2H), 1.4-2.0 (m, 8H), 1.30 (t, 3H).

### EXAMPLE 98

### (E)-3-[4-(2-Ethyl-5,7-dimethyl-imidazo[4,5-b]pyridin-3-ylmethyl)-phenyl]-1-(4-morpholin-4-yl-piperidin-1-yl)-propenone

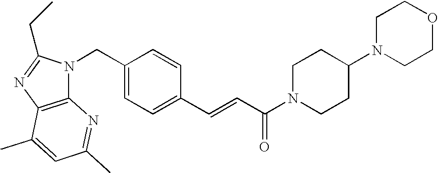

This compound was synthesized analogously to example 23 from acid (6) and 4-piperidin-4-yl-morpholine.

MS (ESI): 488 [M+H]⁺, ¹H-NMR (DMSO-d₆): δ (ppm) 7.65 (d, 2H), 7.40 (d, 1H), 7.23 (d, 1H), 7.14 (d, 2H), 6.96 (s, 1H), 5.47 (s, 2H), 4.44 (m, 1H), 4.24 (m, 1H), 3.56 (t, 4H), 3.05 (m, 1H), 2.79 (q, 2H), 2.66 (m, 1H), 2.55 (s, 3H), 2.50 (s, 3H), 2.45 (t, 4H), 2.41 (m, 1H), 1.80 (m, 2H), 1.29 (m, 2H), 1.23 (t, 3H).

Synthesis of the benzamide Derivatives

The benzamide Derivatives were Generally Prepared by a Synthesis as Lined Out in Reaction Scheme 16.

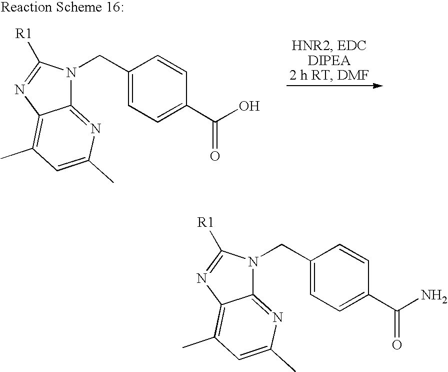

### EXAMPLE 99

### 4-(2-Ethyl-5,7-dimethyl-imidazo[4,5-b]pyridin-3-ylmethyl)-N-(4-pyrrolidin-1-yl-butyl)-benzamide

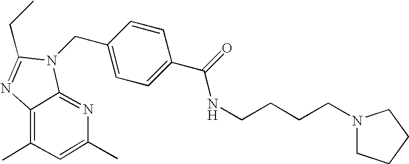

4-(2-Ethyl-5,7-dimethyl-imidazo[4,5-b]pyridin-3-ylmethyl)-benzoic acid (11) (70 mg, 0.226 mmol), 4-pyrrolidinobutylamine (32.2 mg, 0.226 mmol), EDC.HCl (87 mg, 0.453 mmol) and DIPEA (0.119 ml, 0.679 mmol) were dissolved in CH₂Cl₂ (1 ml). The mixture was stirred for 20 h at rt followed by purification by flash chromatography (silica gel, CH₂Cl₂/MeOH 5%→50%) to give the title compound as a colorless oil.

MS (APCI): 434 [M+H]⁺, ¹H-NMR (DMSO-d6, 600 MHz) δ (ppm): 7.77 (d, 2H), 7.21 (d, 2H), 7.03 (s, 1H), 5.61 (s, 2H), 3.40 (t, 2H), 3.15-3.10 (m, 4H), 3.10-3.00 (m, 2H), 2.90-2.80 (m, 2H), 2.61 (s, 3H), 2.57 (s, 3H), 2.05-1.95 (m, 4H), 1.75-1.60 (m, 4H), 1.25 (t, 3H).

### EXAMPLE 100

### N-(4-Azepan-1-yl-butyl)-4-(2-ethyl-5,7-dimethyl-imidazo[4,5-b]pyridin-3-ylmethyl)-benzamide

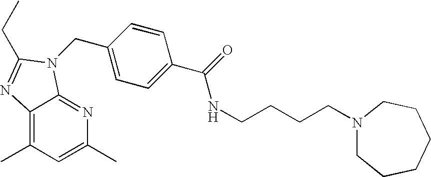

This compound was synthesized analogously to example 99 from acid (11) and (4-azepan-1-ylbutyl)amine.2HCl to give the product as a TFA salt.

MS (APCI): 462 [M+H]⁺, ¹H-NMR (DMSO-d6, 600 MHz) δ (ppm): 7.81 (d, 2H), 7.40 (d, 2H), 7.33 (s, 1H), 5.78 (s, 2H), 3.50-3.10 (m, 10H), 2.67 (s, 3H), 2.65 (s, 3H), 2.05-1.65 (m, 12H), 1.34 (t, 3H).

Synthesis of the anilide Derivatives

The anilide Derivatives were Generally Prepared by a Synthesis as Lined Out in Reaction Scheme 17.

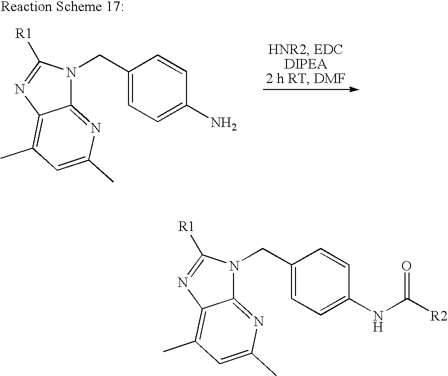

### EXAMPLE 101

### 5-Pyrrolidin-1-yl-pentanoic acid [4-(2-ethyl-5,7-dimethyl-imidazo[4,5-b]pyridin-3-ylmethyl)-phenyl]-amide

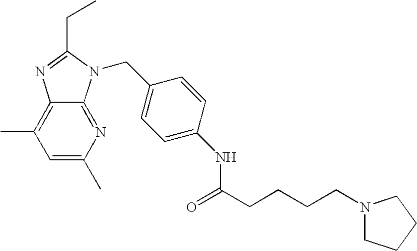

4-(2-Ethyl-5,7-dimethyl-imidazo[4,5-b]pyridin-3-ylmethyl)-phenylamine (14) (234 mg, 0.835 mmol), 5-chlorovaleric acid (114 mg, 0.835 mmol), EDC.HCl (160 mg, 0.835 mmol) and DIPEA (0.146 ml, 0.835 mmol) were dissolved in CH₂Cl₂ (6 ml). The mixture was stirred for 20 h at rt. Then the mixture was directly purified by flash chromatography (silica gel, CH₂Cl₂/MeOH 0%→50%) to give 5-chloro-pentanoic acid [4-(2-ethyl-5,7-dimethyl-imidazo[4,5-b]pyridin-3-ylmethyl)-phenyl]-amide (14b) as a white solid. This material was stirred with excess pyrrolidine (100 eq) for 3 h at 60° C. followed by purification by preparative HPLC (C8, H2O/TFA 900:100:1 to 100:900:1 in 20 min). Fractions containing products were collected and basified with NaHCO₃, before being concentrated. The resulting solution was extracted with EtOAc, washed with brine, dried over Na₂SO₄, filtered and evaporated to dryness to give the title compound as a colorless oil.

MS (APCI): 434 [M+H]⁺, ¹H-NMR (DMSO-d6, 600 MHz) δ (ppm): 7.56 (d, 2H), 7.34 (s, 1H), 7.30 (d, 2H), 5.68 (s, 2H), 3.70-3.60 (m, 2H), 3.20-3.10 (m, 4H), 3.10-3.00 (m, 2H), 2.67 (s, 3H), 2.66 (s, 3H), 2.45 (t, 2H), 2.20-1.95 (m, 4H), 1.80-1.65 (m, 4H), 1.34 (t, 3H).

### EXAMPLE 102

### 5-Piperidin-1-yl-pentanoic acid [4-(2-ethyl-5,7-dimethyl-imidazo[4,5-b]pyridin-3-ylmethyl)-phenyl]-amide

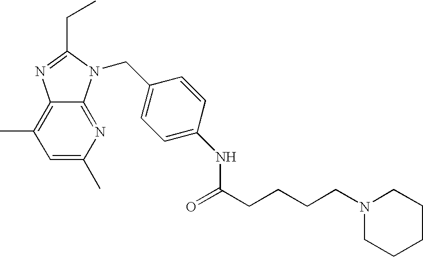

This compound was synthesized analogously to example 100 from aniline 14b and piperidine.

MS (APCI): 448 [M+H]⁺, ¹H-NMR (DMSO-d6, 600 MHz) δ (ppm): 7.56 (d, 2H), 7.34 (s, 1H), 7.30 (d, 2H), 5.69 (s, 2H), 3.60-3.50 (m, 2H), 3.20-3.10 (m, 4H), 3.00-2.90 (m, 2H), 2.68 (s, 3H), 2.66 (s, 3H), 2.46 (t, 2H), 2.00-1.45 (m, 10H), 1.34 (t, 3H).

### EXAMPLE 103

### 5-(3-Dimethylamino-pyrrolidin-1-yl)-pentanoic acid [4-(2-ethyl-5,7-dimethyl-imidazo[4,5-b]pyridin-3-ylmethyl)-phenyl]-amide

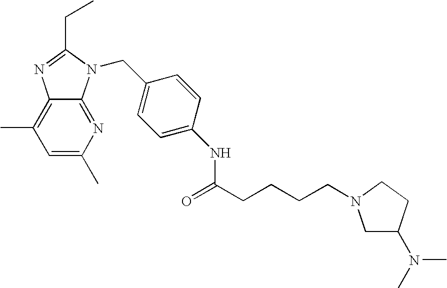

This compound was synthesized analogously to example 100 from aniline 14b and 3-(dimethylamino)pyrolidine.

MS (APCI): 477 [M+H]⁺, ¹H-NMR (DMSO-d6, 600 MHz) δ (ppm): 7.57 (d, 2H), 7.38 (s, 1H), 7.32 (d, 2H), 5.70 (s, 2H), 4.15-4.05 (m, 1H), 3.90-3.40 (m, 6H), 3.21 (q, 2H), 2.96 (s, 6H), 2.69 (s, 3H), 2.66 (s, 3H), 2.65-2.55 (m, 1H), 2.46 (t, 2H), 2.40-2.35 (m, 1H), 1.80-1.60 (m, 4H), 1.34 (t, 3H).

Synthesis of the phenyl-homoallyl amines and 4-phenyl-butyl amines

The phenyl-homoallyl amines and the 4-phenyl-butyl amines and the were Generally Prepared by a one of the Synthesis Routes Outlined in Reaction Scheme 19.

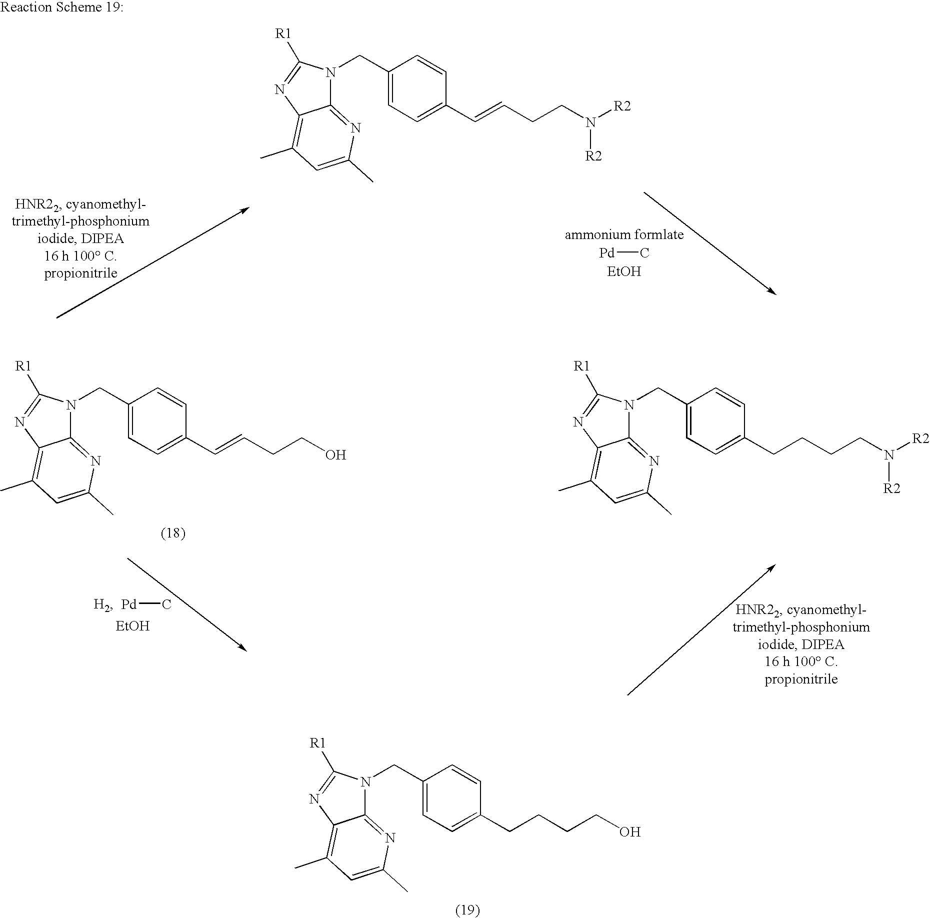

### EXAMPLE 104

### 3-{4-[(E)-4-(4-tert-Butyl-piperazin-1-yl)-but-1-enyl]-benzyl}-2-ethyl-5,7-dimethyl-3H-imidazo[4,5-b]pyridine dihydrochloride

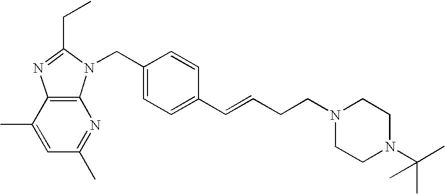

This compound was synthesized analogously to example 6 from alcohol 18 and 1-tert-butyl piperazine.

MS (ESI): 460 [M+H]⁺, ¹H-NMR (DMSO-d6, 500 MHz) δ (ppm): 11.3 (br. s, 2H), 7.36 (d, 2H), 7.15 (m, 3H), 6.51 (d, 1H), 6.25 (m, 1H), 5.52 (s, 2H), 3.90- 2.90 (br. m), 2.55 (s, 3H), 2.53 (s, 3H), 1.35 (s, 9H), 1.22 (t, 3H).

### EXAMPLE 105

### 1′-{(E)-4-[4-(2-Ethyl-5,7-dimethyl-imidazo[4,5-b]pyridin-3-ylmethyl)-phenyl]-but-3-enyl}-[1,4′]bipiperidine

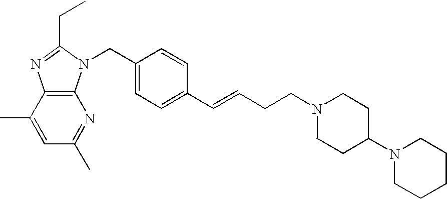

This compound was synthesized analogously to example 6 from alcohol 18 and 4-piperidinopiperidine

MS (ESI): 486 [M+H]⁺, ¹H-NMR (DMSO-d6, 500 MHz) δ (ppm): 11.0 (br. 1H), 10.7 (br. 1H), 7.38 (d, 2H), 7.28 (s, 1H), 7.21 (d, 2H), 6.48 (d, 1H), 6.27 (m, 1H), 5.61 (s, 2H), 3.65-2.78 (m, 12H), 2.61 (s, 3H), 2.57 (s, 3H), 2.36-1.35 (m, 13H), 1.24 (t, 3H).

### EXAMPLE 106

### 2-Ethyl-3-{4-[(E)-4-(4-isopropyl-piperazin-1-yl)-but-1-enyl]-benzyl}-5,7-dimethyl-3H-imidazo[4,5-b]pyridine dihydrochloride

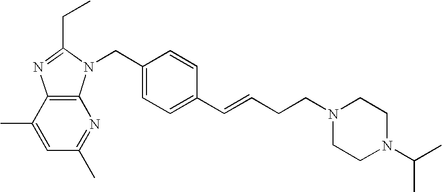

This compound was synthesized analogously to example 6 from alcohol 18 and 1-isopropyl piperazine

MS (ESI): 446 [M+H]⁺, ¹H-NMR (DMSO-d6, 500 MHz) δ (ppm): 12.1 (br. s., 1H), 11.9 (br.s, 1H), 7.39 (d, 2H), 7.27 (s, 1H), 7.21 (d, 2H), 6.51 (d, 1H), 6.28 (m, 1H), 5.61 (s, 2H), 3.76 (br, 2H), 3.66-3.50 (br. m, 6H), 3.24 (br. m, 3H), 3.08 (br. m, 3H), 2.66 (br. m, 1H), 2.61 (s, 3H), 2.57 (s, 3H), 1.27 (d, 6H), 1.24 (t, 3H).

### EXAMPLE 107

### 2-Ethyl-5,7-dimethyl-3-{4-[(E)-4-[4-(4-methyl-piperazin-1-yl)-piperidin-1-yl]-but-1-enyl]-benzyl}-3H-imidazo[4,5-b]pyridine

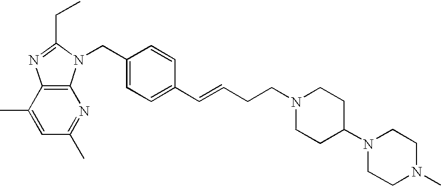

This compound was synthesized analogously to example 6 from alcohol 18 and 1-methyl-4-piperidin-4yl-piperazine

MS (ESI): 460 [M+H]⁺, ¹H-NMR (DMSO-d6, 500 MHz) δ (ppm): 12.1 (br. s., 1H), 11.3 (br.s, 1H), 7.41 (d, 2H), 7.32 (s, 1H), 7.24 (d, 2H), 6.52 (d, 1H), 6.30 (m, 1H), 5.64 (s, 2H), 4.33-3.08 (br. m, 17H), 2.98 (br, 3H), 2.66 (m, 2H), 2.64 (s, 3H), 2.59 (s, 3H), 1.26 (t, 3H).

### EXAMPLE 108

### 2-Ethyl-5,7-dimethyl-3-{4-[(E)-4-(4-morpholin-4-yl-piperidin-1-yl)-but-1-enyl]-benzyl}-3H-imidazo[4,5-b]pyridine dihydrochloride

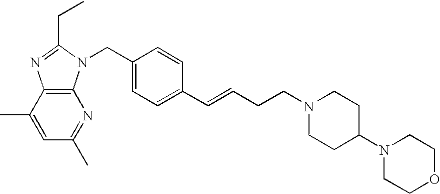

This compound was synthesized analogously to example 6 from alcohol 18 and 4-morpholino piperidine

MS (ESI): 488 [M+H]⁺, ¹H-NMR (DMSO-d6, 500 MHz) δ (ppm): 11.5 (br. s., 1H), 10.8 (br.s, 1H), 7.39 (d, 2H), 7.26 (s, 1H), 7.21 (d, 2H), 6.49 (d, 1H), 6.25 (m, 1H), 5.60 (s, 2H), 3.96 (m, 2H), 3.85 (m, 2H), 3.66 (m, 2H), 3.40 (br. m, 2H), 3.14 (m, 2H), 3.07 (br. m, 2H), 2.96 (m, 2H), 2.65 (m, 2H), 2.61 (s, 3H), 2.57 (s, 3H), 2.33 (m, 2H), 2.12 (m, 2H), 1.24 (t, 3H).

### EXAMPLE 109

### 2-Ethyl-3-[4-((E)-4-imidazo-1-yl)-but-1-enyl)-benzyl]-5,7-dimethyl-3H-imidazo[4,5-b]pyridine

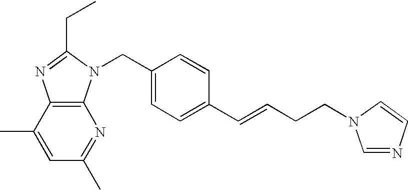

This compound was synthesized analogously to example 80 from 1-butenyl-1H-imidazole and 3-(4-bromo-benzyl)-2-ethyl-5,7-dimethyl-3H-imidazo[4,5-b]pyridine (5h)

MS (ESI): 386 [M+H]⁺, ¹H-NMR (DMSO-d6, 500 MHz) δ (ppm): 9.17 (s, 1H), 7.78 (s, 1H), 7.65 (s, 1H), 7.33 (d, 2H), 7.32 (s, 1H), 7.22 (d, 2H), 6.35 (d, 1H), 6.21 (m, 1H), 5.62 (s, 2H), 4.33 (t, 2H), 3.12 (m, 2H), 2.71 (q, 2H), 2.64 (s, 3H), 2.58 (s, 3H), 1.26 (t, 3H).

### EXAMPLE 110

### 3-{4-[4-(4-tert-Butyl-piperazin-1-yl)-butyl]-benzyl}-2-ethyl-5,7-dimethyl-3H-imidazo[4,5-b]pyridine dihydrochloride

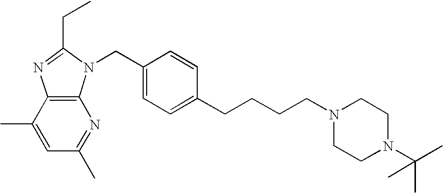

This compound was synthesized from Example 104 by treatment with ammonium formiate and palladium-on-charcoal in ethanol as described for compound 8, followed by purification by preparative HPLC.

MS (ESI): 462 [M+H]⁺, ¹H-NMR (DMSO-d6, 500 MHz) δ (ppm): 11.6 (br. s, 2H), 7.18 (m, 5H), 5.57 (s, 2H), 3.87-3.40 (br. m, 5H) 3.20-2.96 (br. m, 5H), 2.59 (s, 3H), 2.56 (s, 3H) 1.72-1.53 (m, 5H), 1.35 (s, 9H), 1.24 (t, 3H).

### EXAMPLE 111

### 2-Ethyl-3-{4-[4-(4-isopropyl-piperazin-1-yl)-butyl]-benzyl}-5,7-dimethyl-3H-imidazo[4,5-b]pyridine dihydrochloride

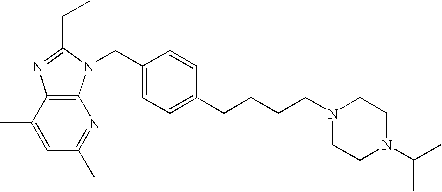

This compound was synthesized analogously to example 6 from alcohol 19 and 1-isopropyl piperazine

MS (ESI): 448 [M+H]⁺, ¹H-NMR (DMSO-d6, 500 MHz) δ (ppm): 7.19 (m, 5H), 5.56 (s, 2H), 4.0-3.35 (br, 9H), 3.12 (br. m, 2H), 3.01 (br. m, 2H), 2.59 (s, 3H), 2.57 (s, 3H), 1.68 (br. m, 2H), 1.57 (m, 2H), 1.27 (m, 9H).

### EXAMPLE 112

### 1′-{4-[4-(2-Ethyl-5,7-dimethyl-imidazo[4,5-b]pyridin-3-ylmethyl)-phenyl]-butyl}-[1,4′]bipiperidine

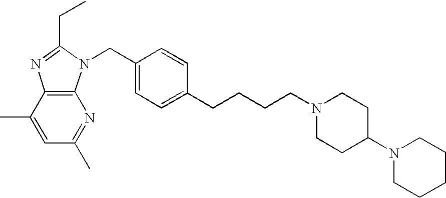

This compound was synthesized analogously to example 6 from alcohol 19 and 4-piperidinopiperidine.

MS (ESI): 488 [M+H]⁺, ¹H-NMR (DMSO-d6, 500 MHz) δ (ppm): 7.10 (d, 2H), 7.02 (d, 2H), 6.94 (s, 1H), 5.40 (s, 2H), 2.76 (m, 2H), 2.73 (q, 2H), 2.49 (2s, 6H), 2.39 (br. m, 4H), 2.17 (m, 2H), 2.08 (br. m, 1H), 1.73 (m, 2H), 1.60 (m, 2H), 1.45 (br. m, 6H), 1.34 (br. m, 6H), 1.19 (t, 3H).

The compounds of formula I in free form or in pharmaceutically acceptable salt form exhibit valuable pharmacological properties, e.g. as GPR4 antagonists as indicated in in vitro tests as described below.

a) Cell-Based Assay for Human GPR4 Activity

HeLa cells stably expressing human GPR4 were established by transfecting the cells with a construct containing the human GPR4 coding sequence. The cells were grown in DMEM/HAM's F12 medium supplemented with 10% fetal calf serum (FCS), 100 u/ml penicillin, 100 μg/ml streptomycin and 400 μg/ml G418 and 10 mM Hepes pH 8.0. pH-induced formation of cAMP was determined using the HTRF technology as provided by CisBio Inc. The cells were seeded in 384-well plates and cultured for 24 hours at 37° C., 5% CO2 before performing the assay. Medium was removed and 10 μl buffer A (HBS, 10 mM Hepes, pH 8, 2 mM IBMX) was added. For compound testing, buffer A with 2× concentrated compounds was used. Cells were incubated for 15 min at room temperature. 10 μl buffer B (HBS, 30 mM Hepes, specific pH) was added to reach the appropriate final pH for stimulation (see below) and incubation was continued for 15 min at room temperature. Finally, 10 μl of cAMP-XL 665 and 10 μl anti cAMP-cryptate were dispensed and plates were read on a Pherastar reader after 60 min incubation at room temperature. Data were calculated from the 665 nm/620 nm ratio and % activity was normalized according to values at minimum and maximum of GPR4 activation. HBS: 130 mM NaCl, 0.9 mM NaH₂PO₄, 5.4 mM KCl, 0.8 mM MgSO₄, CaCl₂ 1.8 mM, 25 mM glucose, 10-30 mM Hepes. Adjustment of HBS buffers: Bf A Bf B Final stimulation pH (1 volume buffer A+1 volume buffer B)

| | |
|---|---|
| pH 8.14 pH 5.68 | 6.92 |
| pH 6.19 | 6.98 |
| pH 6.46 | 7.04 |
| pH 6.86 | 7.19 |
| pH 7.26 | 7.44 |
| pH 7.62 | 7.70 |
| pH 8.00 | 8.00 |
| pH 8.19 | 8.14 |

Compounds were diluted from fresh stock solutions at 10 mM in DMSO to 2 mM and then used for serial dilutions in DMSO. 2× concentrated compound solutions were prepared to reach final concentrations of 20, 6.33, 2, 0.63, 0.2, 0.063, 0.02, 0.0063 uM.

Compounds of formula I have an IC₅₀ between 0.017 and 1 μM:

| | IC50 |
|---|---|
| Ex. | [nM] |
| 1 | 189 |
| 2 | 73 |
| 3 | 584 |
| 4 | 704 |
| 5 | 227 |
| 6 | 395 |
| 7 | 39 |
| 8 | 274 |
| 9 | 341 |
| 10 | 123 |
| 11 | 479 |
| 12 | 53 |
| 13 | 662 |
| 14 | 217 |
| 15 | 50 |
| 16 | 283 |
| 17 | 178 |
| 18 | 186 |
| 19 | 182 |
| 20 | 953 |
| 21 | 345 |
| 22 | 223 |
| 23 | 253 |
| 24 | 961 |
| 25 | 364 |
| 26 | 877 |
| 27 | 411 |
| 28 | 436 |
| 29 | 408 |
| 30 | 94 |
| 31 | 144 |
| 32 | 177 |
| 33 | 71 |
| 34 | 182 |
| 35 | 342 |
| 36 | 477 |
| 37 | 419 |
| 38 | 718 |
| 39 | 280 |
| 40 | 704 |
| 41 | 799 |
| 42 | 162 |
| 43 | 139 |
| 44 | 261 |
| 45 | 46 |
| 46 | 104 |
| 47 | 410 |
| 48 | 194 |
| 49 | 100 |
| 50 | 463 |
| 51 | 161 |
| 52 | 140 |
| 53 | 194 |
| 54 | 166 |
| 55 | 86 |
| 56 | 165 |
| 57 | 121 |
| 58 | 255 |
| 59 | 231 |
| 60 | 995 |
| 61 | 499 |
| 62 | 552 |
| 63 | 829 |
| 64 | 367 |
| 65 | 647 |
| 66 | 222 |
| 67 | 407 |
| 68 | 535 |
| 69 | 201 |
| 70 | 496 |
| 71 | 530 |
| 72 | 452 |
| 73 | 887 |
| 74 | 323 |
| 75 | 146 |
| 76 | 111 |
| 77 | 512 |
| 78 | 114 |
| 79 | 194 |
| 80 | 485 |
| 81 | 758 |
| 82 | 455 |
| 83 | 294 |
| 84 | 181 |
| 85 | 834 |
| 86 | 481 |
| 87 | 296 |
| 88 | 199 |
| 89 | 182 |
| 90 | 568 |
| 91 | 140 |
| 92 | 30 |
| 93 | 29 |
| 94 | 24 |
| 95 | 31 |
| 96 | 994 |
| 97 | 445 |
| 98 | 328 |
| 99 | 997 |
| 100 | 495 |
| 101 | 364 |
| 102 | 241 |
| 103 | 376 |
| 104 | 60 |
| 105 | 115 |
| 106 | 98 |
| 107 | 191 |
| 108 | 447 |
| 109 | 370 |
| 110 | 98 |
| 111 | 412 |
| 112 | 59 |

In a similar manner, assays for the mouse and rat GPR4 receptors have been established. Due to the species specificity of the GPR4 antagonists, the compounds of formula I had an IC₅₀ between 0.3 and 20 μM in the mouse GPR4 assay and between 0.7 and 13 μM in rat GPR4 assay.

b) GPR4 Activity in VEGF-Induced Angiogenesis

The functional activity of GPR4 was determined in the angiogenesis growth factor implant model. Porous tissue chambers made of perfluoro-alkoxy-Teflon were filled with 0.8% agar and 20 U/ml heparin supplemented with or without 8 μg/ml recombinant human VEGF. The solutions were maintained at 39° C. prior to the filling procedure. Mice were anesthetized using 3% isoflurane inhalation. For subcutaneous implantation, a small skin incision was made at the base of the tail to allow the insertion of an implant trocar. The chamber was implanted under aseptic conditions through the small incision onto the back of the animal. The skin incision was closed by wound clips. On the 4th day after implantation, animals were sacrificed using CO₂. Chambers were excised and the vascularized fibrous tissue formed around each implant carefully removed and weighed. Body weight was used to monitor the general condition of the mice. The compounds were applied po at the day of the chamber implantation.

| example | Dose [mg/kg] | | Δ tissue weight [%] |
|---|---|---|---|
| 33 | 10 | (qd) | 45 |
| 46 | 10 | (bid) | 100 |

c) GPR4 Activity in Mouse Antigen-Induced Arthritis Model

Female OFA-1 mice were sensitized i.d. on the back at two sites to methylated bovine serum albumin (mBSA) homogenize 1:1 with complete Freund's adjuvant on days—21 and —14 (0.1 ml containing 1 mg/ml mBSA). On day 0, the right knee receives 10 μl of 10 mg/ml mBSA in 5% glucose solution (antigen-injected knee), while the left knee receives 10 μl of 5% glucose solution alone (vehicle-injected knee). The diameters of the left and right knees were then measured using callipers immediately after the intra-articular injections and again on days 2, 4, 7, 9, 11 and 14. Vehicle and compounds were applied po starting at day 0 for 7 days. Right knee swelling was evaluated relative to left knee swelling, and the R/L knee swelling ratio plotted against time to give area under the curve (AUC) graphs for control and treatment groups. The percentage inhibition of the individual treatment group AUCs were calculated vs the control group AUC (0% inhibition) using an excel spreadsheet. On day 7, the mice were killed by CO₂ inhalation and the right and left knees removed and processed for histological analysis.

| example | Dose [mg/kg] | Δ swelling ratio [%] |
|---|---|---|
| 33 | 50 (bid) | 59 |
| 46 | 100 (bid) | 39 |

d) GPR4 Activity Rat Experimental Autoimmune Uveoretinitis Model

The functional activity of GPR4 was determined in the rat experimental autoimmune uveoretinitis model. Female 6-8 week old Lewis rats were injected in the right footpad with 50 μg purified bovine retinal S—Ag which was dissolved in phosphate-buffered saline, and mixed 1 to 1 with Freund's complete adjuvant and Bacto M Tuberculosis H37 RA adjuvant at day 0. Vehicle and compounds were applied po starting on day 0 and for up to day 21, the eyes were daily inspected with a slit lamp for inflammatory changes. The extent of ocular inflammation was semi-quantitatively assessed with scores from 0 to 4 (0: normal, 1: minimal change in the vasculature, some dilatation of iris and conjunctival blood vessels, 2: moderate change, loss of vascular clearness, dilated iris and blood vessels, cloudy media, 3: marked change, ocular protrusion, obscured pupil, pronounced loss of vascular architecture, some hemorrhage, 4: severe change, marked ocular protrusion, complete loss of architecture, with diffuse hemorrhage).

Typically, compounds of the invention dose-dependently inhibited the clinical scores described above, thereby resulting in a delay of disease onset and a reduction of disease severity.

e) Acute Rat Hyperalgesia Model

Hyperalgesia, induced by an intra-plantar injection of yeast, is measured by applying increasing pressure to the injected foot until the male OFA rats vocalizes or withdraws its foot from the pressure pad. The baseline pressure tolerance is measured at −2 h, followed by a 100 ml injection of 20% yeast in saline into the foot pad. A second measurement at −1 h is taken to demonstrate induction of hyperalgesia (reduced pressure tolerance). The rats were treated orally with a compound of the invention (10, 30 and 90 mg/kg), diclofenac (3 mg/kg) or vehicle (saline, 5 ml/kg., p. o.) at 0 h, and the pressure test repeated 1 and 2 h after dosing. The pressure required to induce vocalization or paw withdrawal of the compound-treated rats at these time-points was compared to that of vehicle treated animals.

In the above described experiment a compound of the invention proofed to be efficacious.

f) Established Rat Hyperalgesia Model

Naïve withdrawal thresholds of both hind paws were determined by using an increasing pressure stimulus placed onto the dorsal surface of each paw using an analgesymeter. Delayed inflammatory pain was then induced by intra-plantar injection of 25 μl of complete Freund's adjuvant (CFA) into one hindpaw with the contralateral paw acting as the control. After 3 days, a compound in accordance to the invention (3, 10, and 30 mg/kg) or diclofenac (30 mg/kg) were administered by gavage as suspension in methylcellulose 5%. One hour later, paw withdrawal thresholds were re-measured on both the ipsilateral (CFA-injected) and contralateral (uninjected) paw; measurements were repeated at 3 hrs and 6 hrs post dosing. The reversal of hyperalgesia was calculated using the following formula: Reversal (%)=100×(postdose ipsilateral threshold−predose ipsilateral threshold) (naïve ipsilateral threshold−predose ipsilateral threshold).

In the above described experiment a compound of the invention proofed to be efficacious.

The term “treatment” as used herein is to be understood as including both therapeutic and prophylactic modes of therapy e.g. in relation to the treatment of neoplasia, therapy to prevent the onset of clinically or preclinically evident neoplasia, or for the prevention of initiation of malignant cells or to arrest or reverse the progression of premalignant to malignant cells, as well as the prevention or inhibition of neoplasia growth or metastasis. In this context, the present invention is, in particular, to be understood as embracing the use of compounds of the present invention to inhibit or prevent development of skin cancer, e.g. squamus or basal cell carcinoma consequential to UV light exposure, e.g. resultant from chronic exposure to the sun.

The compounds of the present invention were in particular useful in the treatment wherein GPR4 inhibition plays a role, for example wherein proton homeostasis is imbalanced, and hence may be useful in treating medical conditions selected from the group consisting of: Osteoporosis (juvenile, menopausal, post-menopausal, post-traumatic, caused by old age or corticosteroid therapy or inactivity), gingivitis, periodontitis, pain, dental pain, Paget's disease, hypercalcemia of malignancy, tumor induced hypercalcemia, metabolic bone disease, cancer, solid tumors, cardiovascular disorders, atherosclerose, myocardial infarction, limb diseases, post thrombotic syndrome (PTS), peripheral arterial occlusive disease, eye diseases, diabetic retinopathy, macular degeneration, uveitis, arthritis, rheumatoid arthritis, osteoarthritis wound healing, skin diseases, inflammatory and obstructive airway diseases, asthma, intrinsic and extrinsic asthma, mild asthma, moderate asthma, severe asthma, bronchitic asthma, exercise induced asthma, occupational asthma and asthma induced following bacterial infection, acute lung injury, acute/adult respiratory distress syndrome, chronic obstructive pulmonary airways or lung diseases, chronic bronchitis, dyspnea associated herewith, emphysema, exacerbation of airways hyperactivity consequent to other drug therapy, bronchitis, acute arachidic, catarrhal, croupus, chronic or phthinoid bronchitis. Pneumoconiosis, aluminosis, anthracosis, asbestosis, chlicosis, ptilosis, siderosis, silicosis, tabacosis byssinosis, eosinophilia, bronchopulmonar aspergillosis, polyarteritis nodosa, eosinophilic granuloma and eosinophil-related disorders affecting the airways occasioned by drug reaction, infections by organisms such as pneumocystis carinii, trypanosoma cruzi, trypanosoma brucei, crithidia fusculata, parasitic diseases such as schistosomiasis and malaria, angiogenesis related diseases, tumor invasion and metastasis, metachromatic leukodystrophy, muscular dystrophy, amythrophy, autoimmune disease, respiratory disease, immunologically mediated disease, transplant rejection.

In another embodiment the compounds of the invention are useful in the treatment of a disorder or disease as being exemplified in the above disclosed animal models, for example in the treatment of angiogenesis, arthritis, uveoretinitis, and/or hyperalgesia.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the present invention can be assessed by the following in vitro & in vivo methods.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents; for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The compounds of the invention may also be administered simultaneously, separately or sequentially in combination with one or more other suitable active agents selected from but not limited to the following classes of agents: Anti IL-1 agents, e.g: Anakinra; anti cytokine and anti-cytokine receptor agents, e.g. anti IL-6 R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.

In another embodiment, the invention provides a method of modulating the GPR4 receptor activity in a subject, wherein the method comprises administering to the subject a therapeutically effective amount of a compound according to the definition of formula (I).

In another embodiment, the invention provides a method of treating a disorder or a disease in a subject mediated by the GPR4 receptor, wherein the method comprises administering to the subject a therapeutically effective amount of a compound according to the definition of formula (I).

In another embodiment, the invention provides a method of treating a disorder or a disease in a subject mediated by the GPR4 receptor, wherein the disorder or the disease is selected from osteoporosis (juvenile, menopausal, post-menopausal, post-traumatic, caused by old age or corticosteroid therapy or inactivity), gingivitis, periodontitis, pain, dental pain, Paget's disease, hypercalcemia of malignancy, tumor induced hypercalcemia, metabolic bone disease, cancer, solid tumors, cardiovascular disorders, atherosclerose, myocardial infarction, limb diseases, post thrombotic syndrome (PTS), peripheral arterial occlusive disease, eye diseases, diabetic retinopathy, macular degeneration, uveitis, arthritis, rheumatoid arthritis, osteoarthritis wound healing, skin diseases, inflammatory and obstructive airway diseases, asthma, intrinsic and extrinsic asthma, mild asthma, moderate asthma, severe asthma, bronchitic asthma, exercise induced asthma, occupational asthma and asthma induced following bacterial infection, acute lung injury, acute/adult respiratory distress syndrome, chronic obstructive pulmonary airways or lung diseases, chronic bronchitis, dyspnea associated herewith, emphysema, exacerbation of airways hyperactivity consequent to other drug therapy, bronchitis, acute arachidic, catarrhal, croupus, chronic or phthinoid bronchitis. Pneumoconiosis, aluminosis, anthracosis, asbestosis, chlicosis, ptilosis, siderosis, silicosis, tabacosis byssinosis, eosinophilia, bronchopulmonar aspergillosis, polyarteritis nodosa, eosinophilic granuloma and eosinophil-related disorders affecting the airways occasioned by drug reaction, infections by organisms such as pneumocystis carinii, trypanosoma cruzi, trypanosoma brucei, crithidia fusculata, parasitic diseases such as schistosomiasis and malaria, angiogenesis related diseases, tumor invasion and metastasis, metachromatic leukodystrophy, muscular dystrophy, amythrophy, autoimmune disease, respiratory disease, immunologically mediated disease, transplant rejection.

In another embodiment, the invention provides a compound according to the definition of formula (I), for use as a medicament.

In another embodiment, the invention provides the use of a compound according to the definition of formula (I), for the treatment of a disorder or disease in a subject mediated by the GPR4 receptor.

In another embodiment, the invention provides the use of a compound according to the definition of formula (I), for the treatment of a disorder or disease in a subject characterized by an activity of the GPR4 receptor.

In another embodiment the compounds according to the invention contain antagonistic activity against the GPR4 receptor.

In another embodiment the invention provides a compound of formula (I), or a pharmaceutically acceptable salt thereof;

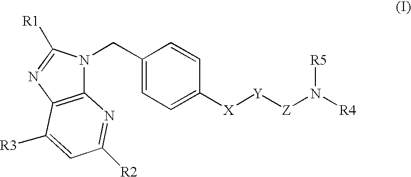

wherein

R1 is lower alkyl optionally substituted by halogen;

R2 and R3 are independently selected from H and lower alkyl;

X—Y stands for —C≡C—, or —CH═CH—, —CH═CHF—, —CH₂—CH₂—, —NHCO—, —CONH—;

Z is —CH₂—, —CH₂—CH₂—, —CH₂—CH₂—CH₂—CH₂—, —CO—, bond;

R4 is H or lower alkyl and R5 is selected from lower alkyl substituted by heterocyclyl;

or R4 and R5 together with the nitrogen atom to which they are attached form a heterocyclic ring;

or R4 and R5 together with the nitrogen atom to which they are attached form a heteroaryl.

In another embodiment the invention provides compound according to formula (I) as defined above, wherein

R1 is lower alkyl optionally substituted by halogen;

R2 and R3 are independently selected from H and lower alkyl;

X—Y stands for —C≡C—, or —CH═CH—, —CH═CHF—, —CH₂—CH₂—, —NHCO—, —CONH—;

Z is —CH₂—, —CH₂—CH₂—, —CH₂—CH₂—CH₂—CH₂—, —CO—, bond;

R4 is H or lower alkyl and R5 is selected from lower alkyl substituted by heterocyclyl;

or R4 and R5 together with the nitrogen atom to which they are attached form a heterocyclic ring which is optionally substituted by lower alkoxy; lower alkoxy substituted by (lower)alkylaminocarbonyl; hydroxyl; di-lower alkyl amino; heterocyclyl; or by lower alkyl optionally substituted by halogen, carbamoyl, alkoxycarbonyl, alkoxycarbonyl amino, hydroxyl, lower alkoxy, amino, di-lower alkyl amino, di-lower alkyl aminocarbonyl, cycloalkyl, aryl or heterocyclyl;

or R4 and R5 together with the nitrogen atom to which they are attached form a heteroaryl.

In another embodiment the invention provides compound according to formula (I) as defined above, wherein

R1 is lower alkyl optionally substituted by halogen;

R2 and R3 are independently selected from H and lower alkyl;

X—Y stands for —C≡C—, or —CH═CH—, —CH═CHF—, —CH₂—CH₂—, —NHCO—, —CONH—;

Z is —CH₂—, —CH₂—CH₂—, —CH₂—CH₂—CH₂—CH₂—, —CO—, bond;

R4 is H or lower alkyl and R5 is selected from lower alkyl substituted by heterocyclyl;

or R4 and R5 together with the nitrogen atom to which they are attached form a heterocyclic ring which is optionally substituted by lower alkoxy; lower alkoxy substituted by (lower)alkylaminocarbonyl; hydroxyl; di-lower alkyl amino; heterocyclyl; or by lower alkyl optionally substituted by halogen, carbamoyl, alkoxycarbonyl, alkoxycarbonyl amino, hydroxyl, lower alkoxy, amino, di-lower alkyl amino, di-lower alkyl aminocarbonyl, cycloalkyl, aryl or heterocyclyl.

In another embodiment the invention provides compound according to formula (I) as defined above, wherein

R1 is C₁-C₄ alkyl; in particular ethyl;

R2 and R3 are independently selected from C₁-C₂ alkyl; in particular methyl;

X—Y stands for —CH₂—CH₂—; —C≡C—, or —CH═CH—; in particular —CH═CH—;

Z is —CH₂— or —CH₂—CH₂—;

R4 and R5 together with the nitrogen atom to which they are attached form a piperidine or a piperazin ring which is optionally substituted in position 4 by C₁-C₆ alkyl, di-C₁-C₄ alkyl amino, 4-C₁-C₆-alkyl-piperazin-1-yl, 4-C₁-C₆-alkyloxy(lower)alkyl-piperazin-1-yl, 4-C₁-C₆-dialkylamino(lower)alkyl-piperazin-1-yl, 1-morpholinyl, 1-piperidinyl, 1-pyrrolidinyl.

In another embodiment the invention provides a compound according to formula (I) as defined above, wherein

R1 is C₁-C₄ alkyl; in particular ethyl;

R2 and R3 are independently selected from C₁-C₂ alkyl; in particular methyl;

X—Y stands for —CH₂—CH₂—; —C≡C—, or —CH═CH—; in particular —CH═CH—;

Z is —CH₂— or —CH₂—CH₂—;

R4 and R5 together with the nitrogen atom to which they are attached form an imidazol-1-yl.

In another embodiment the invention provides a compound according to formula (I) as defined above for use as a pharmaceutical, in particular for use in a disease or disorder being mediated by the GPR4 receptor.

In another embodiment the invention provides method of modulating GPR4 receptor activity in a subject, in particular a method of treating a disorder or a disease in a subject mediated by the GPR4 receptor, wherein the method comprises administering to the subject a therapeutically effective amount of the compound according to formula (I) as defined above.

In another embodiment the invention provides the use of a compound according to formula (I) as defined above, for the treatment of a disorder or disease in a subject mediated by the GPR4 receptor or characterized by an activity of the GPR4 receptor.

In another embodiment the invention provides a method or use of a compound according to formula (I) as defined above in a treatment of a disorder or disease in a subject mediated by the GPR4 receptor or characterized by an activity of the GPR4, wherein said treatment of a disorder or disease is selected from: osteoporosis (juvenile, menopausal, post-menopausal, post-traumatic, caused by old age or corticosteroid therapy or inactivity), gingivitis, periodontitis, pain, dental pain, Paget's disease, hypercalcemia of malignancy, tumor induced hypercalcemia, metabolic bone disease, cancer, solid tumors, cardiovascular disorders, atherosclerose, myocardial infarction, limb diseases, post thrombotic syndrome (PTS), peripheral arterial occlusive disease, eye diseases, diabetic retinopathy, macular degeneration, uveitis, arthritis, rheumatoid arthritis, osteoarthritis wound healing, skin diseases, inflammatory and obstructive airway diseases, asthma, intrinsic and extrinsic asthma, mild asthma, moderate asthma, severe asthma, bronchitic asthma, exercise induced asthma, occupational asthma and asthma induced following bacterial infection, acute lung injury, acute/adult respiratory distress syndrome, chronic obstructive pulmonary airways or lung diseases, chronic bronchitis, dyspnea associated herewith, emphysema, exacerbation of airways hyperactivity consequent to other drug therapy, bronchitis, acute arachidic, catarrhal, croupus, chronic or phthinoid bronchitis. Pneumoconiosis, aluminosis, anthracosis, asbestosis, chlicosis, ptilosis, siderosis, silicosis, tabacosis byssinosis, eosinophilia, bronchopulmonar aspergillosis, polyarteritis nodosa, eosinophilic granuloma and eosinophil-related disorders affecting the airways occasioned by drug reaction, infections by organisms such as pneumocystis carinii, trypanosoma cruzi, trypanosoma brucei, crithidia fusculata, parasitic diseases such as schistosomiasis and malaria, angiogenesis related diseases, tumor invasion and metastasis, metachromatic leukodystrophy, muscular dystrophy, amythrophy, autoimmune disease, respiratory disease, immunologically mediated disease, transplant rejection.

In another embodiment the invention provides a pharmaceutical composition comprising a compound according to the definition of formula (I) as defined above together with a pharmaceutically acceptable carrier.

In another embodiment the invention provides a combination comprising a compound according to the definition of formula (I) as defined above together with one or more other suitable active agents selected from but not limited to the following classes of agents: Anti IL-1 agents, e.g: Anakinra; anti cytokine and anti-cytokine receptor agents, e.g. anti IL-6 R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.

1. A compound of formula (I), or a pharmaceutically acceptable salt thereof; 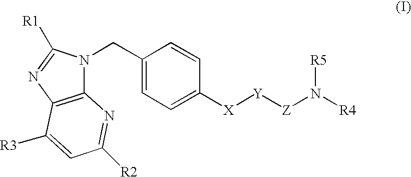
wherein
R1 is lower alkyl optionally substituted by halogen;
R2 and R3 are independently selected from H and lower alkyl;
X—Y stands for —C≡C—, or —CH═CH—, —CH═CHF—, —CH₂—CH₂—, —NHCO—, —CONH—;
Z is —CH₂—, —CH₂—CH₂—, —CH₂—CH₂—CH₂—CH₂—, —CO—, bond;
R4 is H or lower alkyl and R5 is selected from lower alkyl substituted by heterocyclyl;
or R4 and R5 together with the nitrogen atom to which they are attached form a heterocyclic ring;
or R4 and R5 together with the nitrogen atom to which they are attached form a heteroaryl.

2. The compound of claim 1, wherein
R1 is lower alkyl optionally substituted by halogen;
R2 and R3 are independently selected from H and lower alkyl;
X—Y stands for —C≡C—, or —CH═CH—, —CH═CHF—, —CH₂—CH₂—, —NHCO—, —CONH—;
Z is —CH₂—, —CH₂—CH₂—, —CH₂—CH₂—CH₂—CH₂—, —CO—, bond;
R4 is H or lower alkyl and R5 is selected from lower alkyl substituted by heterocyclyl;
or R4 and R5 together with the nitrogen atom to which they are attached form a heterocyclic ring which is optionally substituted by lower alkoxy; lower alkoxy substituted by (lower)alkylaminocarbonyl; hydroxyl; di-lower alkyl amino; heterocyclyl; or by lower alkyl optionally substituted by halogen, carbamoyl, alkoxycarbonyl, alkoxycarbonyl amino, hydroxyl, lower alkoxy, amino, di-lower alkyl amino, di-lower alkyl aminocarbonyl, cycloalkyl, aryl or heterocyclyl;
or R4 and R5 together with the nitrogen atom to which they are attached form a heteroaryl.

3. The compound of claim 1, wherein
R1 is lower alkyl optionally substituted by halogen;
R2 and R3 are independently selected from H and lower alkyl;
X—Y stands for —C≡C—, or —CH═CH—, —CH═CHF—, —CH₂—CH₂—, —NHCO—, —CONH—;
Z is —CH₂—, —CH₂—CH₂—, —CH₂—CH₂—CH₂—CH₂—, —CO—, bond;
R4 is H or lower alkyl and R5 is selected from lower alkyl substituted by heterocyclyl;
or R4 and R5 together with the nitrogen atom to which they are attached form a heterocyclic ring which is optionally substituted by lower alkoxy; lower alkoxy substituted by (lower)alkylaminocarbonyl; hydroxyl; di-lower alkyl amino; heterocyclyl; or by lower alkyl optionally substituted by halogen, carbamoyl, alkoxycarbonyl, alkoxycarbonyl amino, hydroxyl, lower alkoxy, amino, di-lower alkyl amino, di-lower alkyl aminocarbonyl, cycloalkyl, aryl or heterocyclyl.

4. The compound of claim 1, wherein
R1 is C₁-C₄ alkyl;
R2 and R3 are independently selected from C₁-C₂ alkyl;
X—Y stands for —CH₂—CH₂—; —C≡C—, or —CH═CH—;
Z is —CH₂— or —CH₂—CH₂—;
R4 and R5 together with the nitrogen atom to which they are attached form a piperidine or a piperazine ring which is optionally substituted in position 4 by C₁-C₆ alkyl, di-C₁-C₄ alkyl amino, 4-C₁-C₆-alkyl-piperazin-1-yl, 4-C₁-C₆-alkyloxy(lower)alkyl-piperazin-1-yl, 4-C₁-C₆-dialkylamino(lower)alkyl-piperazin-1-yl, 1-morpholinyl, 1-piperidinyl, 1-pyrrolidinyl.

5. The compound of claim 1, wherein
R1 is C₁-C₄ alkyl;
R2 and R3 are independently selected from C₁-C₂ alkyl;
X—Y stands for —CH₂—CH₂—; —C≡C—, or —CH═CH—;
Z is —CH₂— or —CH₂—CH₂—;
R4 and R5 together with the nitrogen atom to which they are attached form an imidazol-1-yl.

6. The compound in accordance to claim 1 for use in a disease or disorder being mediated by the GPR4 receptor.

7. A method of treating a disorder or a disease mediated by the GPR4 receptor in a subject in need of such treatment, wherein the method comprises administering to said subject, a therapeutically effective amount of the compound according to claim 1.

8. (canceled)

9. The Method of claim 7, wherein said disorder or disease is selected from: osteoporosis (juvenile, menopausal, post-menopausal, post-traumatic, caused by old age or corticosteroid therapy or inactivity), gingivitis, periodontitis, pain, dental pain, Paget's disease, hypercalcemia of malignancy, tumor induced hypercalcemia, metabolic bone disease, cancer, solid tumors, cardiovascular disorders, atherosclerose, myocardial infarction, limb diseases, post thrombotic syndrome (PTS), peripheral arterial occlusive disease, eye diseases, diabetic retinopathy, macular degeneration, uveitis, arthritis, rheumatoid arthritis, osteoarthritis wound healing, skin diseases, inflammatory and obstructive airway diseases, asthma, intrinsic and extrinsic asthma, mild asthma, moderate asthma, severe asthma, bronchitic asthma, exercise induced asthma, occupational asthma and asthma induced following bacterial infection, acute lung injury, acute/adult respiratory distress syndrome, chronic obstructive pulmonary airways or lung diseases, chronic bronchitis, dyspnea associated herewith, emphysema, exacerbation of airways hyperactivity consequent to other drug therapy, bronchitis, acute arachidic, catarrhal, croupus, chronic or phthinoid bronchitis. Pneumoconiosis, aluminosis, anthracosis, asbestosis, chlicosis, ptilosis, siderosis, silicosis, tabacosis byssinosis, eosinophilia, bronchopulmonar aspergillosis, polyarteritis nodosa, eosinophilic granuloma and eosinophil-related disorders affecting the airways occasioned by drug reaction, infections by organisms such as pneumocystis carinii, trypanosoma cruzi, trypanosoma brucei, crithidia fusculata, parasitic diseases such as schistosomiasis and malaria, angiogenesis related diseases, tumor invasion and metastasis, metachromatic leukodystrophy, muscular dystrophy, amythrophy, autoimmune disease, respiratory disease, immunologically mediated disease, transplant rejection.

10. A pharmaceutical composition comprising:
the compound according to claim 1 and
a pharmaceutically acceptable carrier.

11. A combination comprising a compound according to claim 1 and
one or more other suitable active agents selected from: Ant IL-1 agents, e.g: Anakinra; anti cytokine and anti-cytokine receptor agents, e.g. anti IL-6 R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.
